(19) United States
(12) Patent Application Publication
(10) Pub. No.: US 2026/0308572 A1
(43) Pub. Date: Oct. 8, 2026

(54) ANALYSIS OF CELLULAR SAMPLES

(71) Applicant: 10x Genomics, Inc., Pleasanton, CA (US)

(72) Inventors: Rajiv BHARADWAJ, Pleasanton, CA (US); Jeremy LACKEY, Foster City, CA (US); Peter SMIBERT, Danville, CA (US)

(21) Appl. No.: 19/637,648

(22) Filed: Apr. 2, 2026

Related U.S. Application Data
(60) Provisional application No. 63/782,519, filed on Apr. 2, 2025.

(51) Int. Cl. C12N 15/10; G01N 33/58; G01N 33/68

(52) U.S. Cl. CPC C12N 15/1065; G01N 33/582; G01N 33/6821; G01N 33/6824

(57) ABSTRACT

The present disclosure relates in some aspects to methods and compositions for analysis of polypeptides from single cells. Polypeptides from a single cell can be uniquely barcoded and identified. Systems and methods directed towards sample preparation for protein sequencing and single cell barcoding allow for massively parallel single-molecule polypeptide sequencing through molecular barcoding and downstream detection and analysis.

### CROSS-REFERENCE TO RELATED APPLICATIONS

This application claims priority to and the benefit of U.S. Provisional Patent Application No. 63/782,519, filed on Apr. 2, 2025, which is hereby incorporated by reference in its entirety.

### FIELD

The present disclosure relates in some aspects to methods and compositions for analysis of polypeptides from single cells. Polypeptides from a single cell are processed to be uniquely barcoded and identified. Systems and methods directed to sample preparation for protein sequencing and single cell barcoding allow for massively parallel single-molecule polypeptide sequencing through molecular barcoding and downstream detection and analysis.

### BACKGROUND

A sample may be processed for various purposes, such as for identification of analytes within the sample. Compositions and methods related to the analysis of nucleic acids in single cells provide useful genomic and transcriptomic data at the single cell level. Proteins play an important role in cell biology and physiology but many protein analysis approaches rely on indirect and limited antibody based methods. Such approaches are not amenable to analysis of the whole proteome expressed in each single cell. Additional compositions and methods for analysis of protein expression at the single cell level are needed. Provided herein are methods and compositions that address such and other needs.

### SUMMARY

Provided herein is a method comprising providing an emulsion comprising a droplet population, wherein a droplet of the droplet population comprises i) a plurality of digested polypeptides from a single cell, and ii) a nucleic acid barcode molecule coupled to a particle; subjecting the emulsion to conditions sufficient to transfer a conjugation reagent to the droplet population via micelles; and using a reducing agent to release the nucleic acid barcode molecule coupled to the particle; thereby generating a polypeptide-oligonucleotide conjugate by coupling a digested polypeptide of the plurality of digested polypeptides to the nucleic acid barcode molecule.

In some embodiments, the nucleic acid barcode molecule is attached to a lysine of the digested polypeptide to form the polypeptide-oligonucleotide conjugate. In some embodiments, the nucleic acid barcode molecule is coupled to an internal amino acid of the digested polypeptide. In some instances, the nucleic acid barcode molecule is covalently coupled to the digested polypeptide. In some instances, the coupling of the nucleic acid barcode molecule to the digested polypeptide is copper catalyzed. In some instances, prior to coupling the nucleic acid barcode molecule to the digested polypeptide, the digested polypeptide is modified to comprise an azide group. In some instances, the modification comprises contacting the digested polypeptide with a diazo transfer reagent in the presence of a copper salt and a base. In some instances, the copper salt is CuSO₄. In some instances, the base is K₂CO₃. In some instances, the diazo transfer reagent is imidazole-1-sulfonyl azide hydrochloride. In some instances, unreacted diazo transfer reagent is contacted with a cyclooctyne to quench the unreacted diazo transfer reagent. In some instances, the cyclooctyne is a dibenzocyclooctyne. In some instances, the quenching proceeds by way of strain-promoted alkyne-azide cycloaddition (SPAAC). In some embodiments, prior to coupling the nucleic acid barcode molecule to the digested polypeptide, the nucleic acid barcode molecule is modified to comprise an alkyne group. In some embodiments, the modification comprises contacting the nucleic acid barcode molecule with 5-octadinyl dU. In some embodiments, the coupling of the nucleic acid barcode molecule to the digested polypeptide proceeds by way of copper-catalyzed alkyne-azide cycloaddition (CuAAC).

In some embodiments, the droplet of the droplet population comprises a plurality of nucleic acid barcode molecules comprising a particle-specific barcode common to all nucleic acid barcode molecules coupled to the same particle. In some embodiments, the plurality of digested polypeptides in the droplet of the droplet population are coupled to the plurality of nucleic acid barcode molecules comprising the common particle-specific barcode. In some embodiments, the nucleic acid barcode molecule comprises a functional sequence. In some embodiments, the nucleic acid barcode molecule comprises a Unique Molecular Identifier (UMI).

In some embodiments, energy is applied to couple the digested polypeptide to the nucleic acid barcode molecule. In some instances, the energy applied comprises agitation. In some instances, the energy applied comprises heat. In some embodiments, the energy is applied in the presence of a copper salt to catalyze coupling of the digested polypeptide to the nucleic acid barcode molecule. In some instances, the copper salt is CuSO₄. In some embodiments, the digested polypeptide comprises an azide group and the nucleic acid barcode molecule comprises an alkyne group, and wherein the azide group and the alkyne group are coupled via a copper-catalyzed alkyne-azide cycloaddition reaction to form a triazole group. In some instances, the reducing agent is a disulfide cleaving reagent.

In some embodiments, the reducing agent is selected from the group consisting of 2-mercaptoethanol (BME), tris(2-carboxyethyl) phosphine hydrochloride (TCEP), and dithiothreitol (DTT), or a functional derivative thereof. In some embodiments, the reducing agent is dithiothreitol (DTT), or a functional derivative thereof. In some embodiments, the reducing agent and/or the conjugation reagent is in a water-in-oil emulsion and the micelles are water-in-oil micelles. In some instances, the micelles are water-in-fluorinated oil micelles. In some embodiments, the micelles are formed using a surfactant.

In some embodiments, the reducing agent and/or the conjugation reagent is provided to the droplet population using an additional emulsion comprising the reducing agent and/or the conjugation reagent. In some embodiments, the additional emulsion comprises additional droplets comprising the reducing agent and/or the conjugation reagent. In some embodiments, the conjugation reagent is transferred to the droplet population prior to the releasing the nucleic acid barcode molecules from the particle. In some embodiments, the conjugation reagent is transferred to the droplet population with the reducing agent. In some embodiments, the reducing agent is transferred to the droplet population via micelles. In some embodiments, the reducing agent is transferred to the droplet population with copper via micelles. In some embodiments, the reducing agent is transferred to the droplet population via micelles after the conjugation reagent is transferred to the droplet population.

In some instances, the reducing agent dissolves the particle and/or releases at least a portion of the plurality of nucleic acid barcode molecules from the particle. In some instances, the conjugation reagent is selected from the group consisting of 1-Ethyl-3-(3-dimethylaminopropyl) carbodiimide (EDC), dicyclohexylcarbodiimide (DCC), and diisopropylcarbodiimide (DIC). In some instances, the conjugation reagent is dicyclohexylcarbodiimide (DCC).

In some embodiments, the conjugation reagent facilitates coupling of the digested polypeptide to the nucleic acid barcode molecule through coupling of the C-terminus of the digested polypeptide to an amine on the nucleic acid barcode molecule. In some embodiments, the single cell is lysed in the droplet of the droplet population.

In some instances, the method further comprises denaturing proteins in the droplet of the droplet population. In some embodiments, the droplet of the droplet population comprises a lysis agent. In some embodiments, the plurality of digested polypeptides from the single cell are generated by treating the droplet population with an enzyme. In some embodiments, the enzyme denatures proteins in the single cell to form the digested polypeptides. In some embodiments, the enzyme is a digestion enzyme. In some instances, the digestion enzyme is trypsin.

In some embodiments, the plurality of polypeptide-oligonucleotide conjugates are immobilized onto a surface. In some embodiments, the plurality of polypeptide-oligonucleotide conjugates are released from the emulsion prior to immobilizing the plurality of polypeptide-oligonucleotide conjugates to the surface. In some instances, the surface is a bead. In some instances, the bead is a polystyrene bead, a polymer bead, an agarose bead, an acrylamide bead, a solid core bead, a porous bead, a paramagnetic bead, glass bead, or a controlled pore bead. In some instances, the polypeptide-oligonucleotide conjugate is immobilized to the surface via a thiol group. In some embodiments, the polypeptide-oligonucleotide conjugate is immobilized to the surface by Michael-type addition of a thiol to a maleimide group. In some instances, the thiol is a thiol in a cysteine group in the digested polypeptide of the polypeptide-oligonucleotide conjugate.

In some embodiments, the sequence of the digested polypeptide and the sequence of the nucleic acid barcode molecule of the polypeptide-oligonucleotide conjugate or a derivative thereof is determined. In some embodiments, determining the sequence of the digested polypeptide comprises contacting the digested polypeptide of the polypeptide-oligonucleotide conjugate with a binding agent comprising a coding tag sequence associated with the binding agent; and extending the nucleic acid barcode molecule of the polypeptide-oligonucleotide conjugate with the coding tag sequence or a complement thereof, thereby generating an extended nucleic acid barcode molecule. In some embodiments, the binding agent is specific to the N-terminal amino acid residue of the digested polypeptide or a derivative thereof. In some embodiments, a terminal amino acid or terminal amino acid derivative is cleaved from the digested polypeptide. In some embodiments, additional binding agents are contacted with the digested polypeptide and the extended nucleic acid barcode molecule is extended with one or more additional coding tag sequences. In some embodiments, the extended nucleic acid barcode molecule identifies the digested polypeptide and the single cell where the digested polypeptide originated.

In some embodiments, determining the sequence of the nucleic acid barcode molecule comprises sequencing. In some embodiments, the extended nucleic acid barcode molecule or a derivative thereof comprising (i) the coding tag sequence or a complement thereof and (ii) a sequence of the nucleic acid barcode or a complement thereof is sequenced.

In some embodiments, the particle is a bead. In some embodiments, the bead comprises a degradable bead. In some examples, the bead is a gel bead. In some embodiments, the droplets from the droplet population are generated using a microfluidic device. In some embodiments, the droplet population is generated upon agitation of a mixture of immiscible phases. In some embodiments, the droplet population is generated from (i) a first fluid volume comprising a population of particles and a population of cells and (ii) a second fluid volume immiscible with the first fluid volume, wherein the droplet of the droplet population comprises the particle from the population of particles and the single cell from the population of cells.

Provided herein is a method comprising providing a plurality of digested polypeptides from a single cell in a partition, wherein the partition comprises a plurality of nucleic acid barcode molecules unique to the partition; providing a reducing agent and a conjugation reagent to the partition to generate a plurality of polypeptide-oligonucleotide conjugates by coupling the plurality of digested polypeptides to the plurality of nucleic acid barcode molecules wherein each polypeptide-oligonucleotide conjugate of the plurality of polypeptide-oligonucleotide conjugates comprises a digested polypeptide of the plurality of digested polypeptides coupled to a nucleic acid barcode molecule of the plurality of nucleic acid barcode molecules; dispersing the plurality of polypeptide-oligonucleotide conjugates on a substrate; and detecting, for a polypeptide-oligonucleotide conjugate of the plurality of polypeptide-oligonucleotide conjugates at a location on the substrate, a plurality of signals comprising (i) a first signal associated with the digested polypeptide of the polypeptide-oligonucleotide conjugate, and (ii) a second signal associated with the nucleic acid barcode molecule of the polypeptide-oligonucleotide conjugate.

In some embodiments, the method comprises detecting a plurality of signals for each of two or more polypeptide-oligonucleotide conjugates of the plurality of polypeptide-oligonucleotide conjugates. In some instances, the two or more polypeptide-oligonucleotide conjugates are positioned at different locations on the substrate, wherein the plurality of signals for each of the two or more polypeptide-oligonucleotide conjugates are detected from different locations on the substrate. In some instances, a substrate comprises a first polypeptide-oligonucleotide conjugate at a first location and a second polypeptide-oligonucleotide conjugate at a second location. In some instances, the detecting comprises detecting a first plurality of signals comprising (i) a first signal associated with the digested polypeptide of the first polypeptide-oligonucleotide conjugate, and (ii) a second signal associated with the nucleic acid barcode molecule of the first polypeptide-oligonucleotide conjugate and a second plurality of signals comprising (i) a first signal associated with the digested polypeptide of the second polypeptide-oligonucleotide conjugate, and (ii) a second signal associated with the nucleic acid barcode molecule of the second polypeptide-oligonucleotide conjugate. In some instances, the first signal associated with the digested polypeptide of a polypeptide-oligonucleotide conjugate is part of an optical signature associated with the digested polypeptide. In some instances, the second signal associated with the digested polypeptide of a polypeptide-oligonucleotide conjugate is part of an optical signature associated with the origin of polypeptide.

In some embodiments, the nucleic acid barcode molecule is attached to a lysine of the digested polypeptide to form the polypeptide-oligonucleotide conjugate. In some instances, in each polypeptide-oligonucleotide conjugate of the plurality of polypeptide-oligonucleotide conjugates, the nucleic acid barcode molecule is coupled to an internal amino acid of the digested polypeptide. In some instances, in each polypeptide-oligonucleotide conjugate of the plurality of polypeptide-oligonucleotide conjugates, the nucleic acid barcode molecule is covalently coupled to the digested polypeptide. In some embodiments, the coupling of the nucleic acid barcode molecule to the digested polypeptide is copper catalyzed. In some embodiments, prior to coupling the nucleic acid barcode molecule to the digested polypeptide, the digested polypeptide is modified to comprise an azide group. In some embodiments, the modification comprises contacting the digested polypeptide with a diazo transfer reagent in the presence of a copper salt and a base. In some cases, the copper salt is CuSO₄. In some cases, the base is K₂CO₃. In some instances, the diazo transfer reagent is imidazole-1-sulfonyl azide hydrochloride. In some embodiments, unreacted diazo transfer reagent is contacted with a cyclooctyne to quench the unreacted diazo transfer reagent. In some instances, the cyclooctyne is a dibenzocyclooctyne. In some embodiments, the quenching proceeds by way of strain-promoted alkyne-azide cycloaddition (SPAAC). In some embodiments, prior to coupling the nucleic acid barcode molecule to the digested polypeptide, the nucleic acid barcode molecule is modified to comprise an alkyne group. In some instances, the modification comprises contacting the nucleic acid barcode molecule with 5-octadinyl dU. In some embodiments, in each polypeptide-oligonucleotide conjugate of the plurality of polypeptide-oligonucleotide conjugates, the nucleic acid barcode molecule is coupled to the digested polypeptide by copper-catalyzed alkyne-azide cycloaddition (CuAAC). In some embodiments, a copper salt is used to catalyze coupling of the digested polypeptide to the nucleic acid barcode molecule. In some instances, the copper salt is CuSO₄.

In some embodiments, the nucleic acid barcode molecule comprises a functional sequence and/or a Unique Molecular Identifier (UMI).

In some embodiments, the digested polypeptide comprises an azide group and the nucleic acid barcode molecule comprises an alkyne group, and wherein the azide group and the alkyne group are coupled via a copper-catalyzed alkyne-azide cycloaddition reaction to form a triazole group. In some embodiments, the reducing agent is a disulfide cleaving reagent. In some instances, the reducing agent is selected from the group consisting of 2-mercaptoethanol (BME), tris (2-carboxyethyl) phosphine hydrochloride (TCEP), and dithiothreitol (DTT), or a functional derivative thereof. In some instances, the reducing agent is dithiothreitol (DTT), or a functional derivative thereof. In some embodiments, the single cell is lysed in the partition prior to providing the reducing agent and/or the conjugation reagent. In some instances, the conjugation reagent is selected from the group consisting of 1-Ethyl-3-(3-dimethylaminopropyl) carbodiimide (EDC), dicyclohexylcarbodiimide (DCC), and diisopropylcarbodiimide (DIC). In some examples, the conjugation reagent is dicyclohexylcarbodiimide (DCC). In some embodiments, the conjugation reagent facilitates coupling of the digested polypeptide to the nucleic acid barcode molecule through coupling of the C-terminus of the digested polypeptide to an amine on the nucleic acid barcode molecule.

In some embodiments, a plurality of proteins is treated with an enzyme in the partition to provide the plurality of digested polypeptides. In some instances, the enzyme is a digestion enzyme. In some instances, the digestion enzyme is trypsin.

In some embodiments, the plurality of nucleic acid barcode molecules is joined to a support. In some instances, the support is a bead. In some instances, the plurality of nucleic acid barcode molecules is releasable from the support upon application of a stimulus. In some examples, the stimulus is a biological stimulus, a chemical stimulus, a thermal stimulus, an electrical stimulus, a magnetic stimulus, and/or a photo stimulus.

In some embodiments, the generated polypeptide-oligonucleotide conjugates are pooled with additional generated polypeptide-oligonucleotide conjugates from a plurality of partitions and the pooled polypeptide-oligonucleotide conjugates are dispersed on the substrate. In some embodiments, the polypeptide-oligonucleotide conjugate is immobilized to the substrate by Michael-type addition of a thiol to a maleimide group. In some embodiments, the thiol is a thiol in a cysteine group in the digested polypeptide of the polypeptide-oligonucleotide conjugate.

In some embodiments, a sequence of the digested polypeptide of the polypeptide-oligonucleotide conjugate and a sequence of the nucleic acid barcode molecule of the polypeptide-oligonucleotide conjugate are determined. In some instances, determining the sequence of the digested polypeptide of the polypeptide-oligonucleotide conjugate comprises detecting a plurality of signals associated with the digested polypeptide of the polypeptide-oligonucleotide conjugate. In some instances, determining the sequence of the digested polypeptide comprises contacting the terminal amino acid of the digested polypeptide of the polypeptide-oligonucleotide conjugate or a derivative thereof with a binding agent. In some embodiments, the binding agent is specific to the N-terminal amino acid residue of the digested polypeptide or a derivative thereof. In some embodiments, the binding agent is detectably labeled. In some embodiments, the binding agent comprises a fluorophore. In some embodiments, determining the sequence of the digested polypeptide of the polypeptide-oligonucleotide conjugate comprises detecting a cleaved terminal amino acid or terminal amino acid derivative from the digested polypeptide.

In some embodiments, determining a sequence of the nucleic acid barcode molecule of the polypeptide-oligonucleotide conjugate comprises amplifying the sequence of the nucleic acid barcode molecule. In some embodiments, the amplifying is performed by rolling circle amplification (RCA). In some embodiments, the nucleic acid barcode molecule of the polypeptide-oligonucleotide conjugate on the substrate is contacted with a circularizable probe, wherein the circularizable probe comprises a 5′ hybridization region and a 3′ hybridization region that binds a first common sequence and a second common sequence, respectively, in the nucleic acid barcode molecule. In some instances, the ends of the circularizable probe are ligated to each other to form a circularized probe, wherein the ligation is preceded by gap filling. In some instances, the circularizable probe is a padlock probe. In some embodiments, the method comprises gap filling the circularizable probe using a cell barcode sequence of the nucleic acid barcode molecule of the polypeptide-oligonucleotide conjugate as a template, thereby incorporating a complement of the cell barcode sequence into the circularizable probe. In some embodiments, RCA is performed by extending the 3′ end of the nucleic acid barcode molecule of the polypeptide-oligonucleotide conjugate on the substrate, wherein the 3′ end serves as a primer to generate an RCA product. In some embodiments, the nucleic acid barcode molecule of the polypeptide-oligonucleotide conjugate or an extension thereof on the substrate is circularized and used as a template for RCA to generate an RCA product.

In some embodiments, determining the sequence of the digested polypeptide comprises contacting the digested polypeptide of the polypeptide-oligonucleotide conjugate with a binding agent comprising a coding tag sequence associated with the binding agent; and extending the nucleic acid barcode molecule of the polypeptide-oligonucleotide conjugate with the coding tag sequence or a complement thereof, thereby generating an extended nucleic acid barcode molecule. In some embodiments, the binding agent is specific to the N-terminal amino acid residue of the digested polypeptide or a derivative thereof. In some embodiments, a terminal amino acid or terminal amino acid derivative is cleaved from the digested polypeptide. In some embodiments, additional binding agents are used, and the extended nucleic acid barcode molecule is further extended with one or more additional coding tag sequences. In some embodiments, the extended nucleic acid barcode molecule identifies the digested polypeptide of the polypeptide-oligonucleotide conjugate and the single cell from which the digested polypeptide originated.

In some embodiments, a sequence of the extended nucleic acid barcode molecule or a derivative thereof comprising (i) the coding tag sequence or a complement thereof and (ii) a sequence of the nucleic acid barcode or a complement thereof is detected. In some embodiments, detecting the plurality of signals associated with the nucleic acid barcode molecule of the polypeptide-oligonucleotide conjugate comprises performing sequencing by ligation, sequencing by synthesis, sequencing by binding, sequencing by avidity, or a combination thereof. In some instances, a sequence of the RCA product is detected by binding a sequencing primer to the RCA product. In some embodiments, the method comprises performing imaging to detect a sequence of the RCA product at the location on the substrate. In some embodiments, detecting the plurality of signals associated with the nucleic acid barcode molecule comprises performing sequencing by synthesis (SBS). In some embodiments, detecting the plurality of signals associated with the nucleic acid barcode molecule comprises performing sequencing by ligation (SBL). In some embodiments, the sequence of the RCP or a complement thereof is sequenced using sequencing by binding (SBB). In some aspects, two or more signals are sequentially detected at a location of the polypeptide-oligonucleotide conjugate from two or more detectably labeled probes that sequentially bind to the nucleic acid barcode molecule or a derivative thereof. In some aspects, two or more signals are sequentially detected at a location of the polypeptide-oligonucleotide conjugate from two or more detectably labeled nucleotides or polymer-nucleotide conjugates for sequencing the nucleic acid barcode molecule or a derivative thereof.

Provided herein is a method comprising providing a plurality of cell beads and a plurality of barcode beads, wherein (i) a cell bead of the plurality of cell beads comprises a plurality of polypeptides from a single cell, and (ii) a barcode bead of the plurality of barcode beads comprises a plurality of nucleic acid barcode molecules for barcoding the plurality of polypeptides, wherein the cell bead is provided with an enzyme to generate a digested polypeptide; contacting the digested polypeptide with a conjugation reagent; and partitioning the plurality of cell beads and the plurality of barcode beads into a plurality of partitions, wherein upon partitioning, a partition of the plurality of partitions comprises the cell bead, the barcode bead and a reducing agent to release a nucleic acid barcode molecule of the plurality of nucleic acid barcode molecule coupled to the barcode bead; thereby generating a polypeptide-oligonucleotide conjugate comprising the digested polypeptide and the nucleic acid barcode molecule.

In some embodiments, the cell bead includes or is enclosed within a gel or polymer matrix within the partition. In some embodiments, the barcode bead includes or is enclosed within a gel or polymer matrix within the partition. In some embodiments, the plurality of partitions is a plurality of droplets. In some embodiments, the plurality of partitions is a plurality of wells. In some embodiments, one or more nucleic acid barcode molecules of the plurality of nucleic acid barcode molecules are coupled to a surface of the barcode bead and/or enclosed within the barcode bead.

In some embodiments, the partition further comprises additional reagents. In some instances, the nucleic acid barcode molecule is attached to a lysine of the digested polypeptide to form the polypeptide-oligonucleotide conjugate. In some embodiments, the nucleic acid barcode molecule is coupled to an internal amino acid of the digested polypeptide. In some embodiments, the nucleic acid barcode molecule is covalently coupled to the digested polypeptide. In some embodiments, the coupling of the nucleic acid barcode molecule to the digested polypeptide is copper catalyzed. In some embodiments, prior to coupling the nucleic acid barcode molecule to the digested polypeptide, the digested polypeptide is modified to comprise an azide group. In some embodiments, the modification comprises contacting the digested polypeptide with a diazo transfer reagent in the presence of a copper salt and a base. In some instances, the copper salt is CuSO₄. In some instances, the base is K₂CO₃. In some embodiments, the diazo transfer reagent is imidazole-1-sulfonyl azide hydrochloride. In some embodiments, unreacted diazo transfer reagent is contacted with a cyclooctyne to quench the unreacted diazo transfer reagent. In some instances, the cyclooctyne is a dibenzocyclooctyne. In some embodiments, the quenching proceeds by way of strain-promoted alkyne-azide cycloaddition (SPAAC). In some embodiments, prior to coupling the nucleic acid barcode molecule to the digested polypeptide, the nucleic acid barcode molecule is modified to comprise an alkyne group. In some embodiments, the modification comprises contacting the nucleic acid barcode molecule with 5-octadinyl dU. In some embodiments, the coupling of the nucleic acid barcode molecule to the digested polypeptide proceeds by way of copper-catalyzed alkyne-azide cycloaddition (CuAAC).

In some embodiments, a copper salt is used to catalyze coupling of the digested polypeptide to the nucleic acid barcode molecule. In some embodiments, the copper salt is CuSO₄. In some embodiments, the digested polypeptide comprises an azide group and the nucleic acid barcode molecule comprises an alkyne group, and wherein the azide group and the alkyne group are coupled via a copper-catalyzed alkyne-azide reaction to form a triazole group. In some embodiments, the reducing agent is a disulfide cleaving reagent. In some embodiments, the reducing agent is selected from the group consisting of 2-mercaptoethanol (BME), tris(2-carboxyethyl) phosphine hydrochloride (TCEP), and dithiothreitol (DTT), or a functional derivative thereof. In some embodiments, the reducing agent is dithiothreitol (DTT), or a functional derivative thereof.

In some embodiments, the single cell is lysed in the cell bead prior to providing the reducing agent and/or the conjugation reagent. In some embodiments, the conjugation reagent is selected from the group consisting of 1-Ethyl-3-(3-dimethylaminopropyl) carbodiimide (EDC), dicyclohexylcarbodiimide (DCC), and diisopropylcarbodiimide (DIC). In some embodiments, the conjugation reagent is dicyclohexylcarbodiimide (DCC). In some embodiments, the conjugation reagent facilitates coupling of the digested polypeptide to the nucleic acid barcode molecule through coupling of the C-terminus of the digested polypeptide to an amine on the nucleic acid barcode molecule. In some instances, the nucleic acid barcode molecules are coupled to the digested polypeptide using an amine bioconjugation chemistry. In some instances, activated DNA of nucleic acid barcode molecules comprises amine-reactive chemistries such as ethynyl moieties or N-hydroxy succinimide (NHS) moieties. In some instances, lysine amines are labeled on the digested polypeptide. In some instances, click-enabled heterobifunctional linkers comprised of an amine-reactive component, and a click chemistry component are used to first activate the lysine amines on the digested polypeptide, and in a second step, a nucleic acid barcode molecule is covalently attached using click chemistry. Examples of click chemistries include CuAAC, SPAAC, and iEDDA chemistries.

In some embodiments, a plurality of proteins is treated with an enzyme in the cell bead to provide the plurality of digested polypeptides. In some embodiments, the nucleic acid barcode molecule comprises a functional sequence and/or a Unique Molecular Identifier (UMI). In some embodiments, the polypeptide-oligonucleotide conjugate is recovered from the partition. In some embodiments, a plurality of polypeptide-oligonucleotide conjugates is pooled from the plurality of partitions. In some instances, the pooled polypeptide-oligonucleotide conjugates are dispersed on a substrate. In some embodiments, the substrate is a bead. In some instances, the bead is a polystyrene bead, a polymer bead, an agarose bead, an acrylamide bead, a solid core bead, a porous bead, a paramagnetic bead, glass bead, or a controlled pore bead. In some embodiments, the polypeptide-oligonucleotide conjugate is immobilized to the substrate. by Michael-type addition of a thiol to a maleimide group. In some examples, the thiol is a thiol in a cysteine group in the digested polypeptide of the polypeptide-oligonucleotide conjugate.

In some embodiments, sequence of the digested polypeptide and the sequence of the nucleic acid barcode molecule of the polypeptide-oligonucleotide conjugate or a derivative thereof is determined. In some embodiments, determining the sequence of the digested polypeptide comprises: contacting the digested polypeptide of the polypeptide-oligonucleotide conjugate with a binding agent comprising a coding tag sequence associated with the binding agent; and extending the nucleic acid barcode molecule of the polypeptide-oligonucleotide conjugate with the coding tag sequence or a complement thereof, thereby generating an extended nucleic acid barcode molecule. In some embodiments, the binding agent is specific to the N-terminal amino acid residue of the digested polypeptide or a derivative thereof. In some embodiments, a terminal amino acid or terminal amino acid derivative is cleaved from the digested polypeptide. In some instances, additional binding agents are used and the extended nucleic acid barcode molecule is extended with one or more additional coding tag sequences. In some embodiments, the extended nucleic acid barcode molecule identifies the digested polypeptide and the single cell where the digested polypeptide originated.

In some embodiments, determining the sequence of the nucleic acid barcode molecule comprises sequencing. In some embodiments, the extended nucleic acid barcode molecule or a derivative thereof comprising (i) the coding tag sequence or a complement thereof and (ii) a sequence of the nucleic acid barcode or a complement thereof is sequenced. In some embodiments, determining the sequence of the digested polypeptide and the sequence of the nucleic acid barcode molecule of the polypeptide-oligonucleotide conjugate or a derivative thereof is performed on the substrate.

In some embodiments, the cell bead is generated by providing a plurality of cells and a plurality of polymeric or gel precursors; partitioning the plurality of cells and the plurality of polymeric or gel precursors into a plurality of partitions, wherein upon partitioning, a partition of the plurality of partitions comprises a cell of the plurality of cells and at least a portion of the polymeric or gel precursors; and subjecting the partitions to conditions suitable for cross-linking or polymerizing the polymeric or gel precursors to generate the cell bead, wherein the cell bead encapsulates the cell.

In some embodiments, the single cell is from a biological sample. In some embodiments, the biological sample is dissociated into a plurality of cells comprising the single cell. In some instances, the plurality of cells are dissociated from the biological sample into single cells using one or more microfluidic channels. In some embodiments, the biological sample is a cell culture sample. In some embodiments, the biological sample is a tissue sample.

Provided herein is a method comprising: providing a partition comprising: i) a digested polypeptide derived from a cell, ii) a nucleic acid derived from the cell, and iii) a particle coupled to a first nucleic acid barcode molecule and a second nucleic acid barcode molecule; selectively releasing the first nucleic acid barcode molecule from the particle in the partition, wherein the second nucleic acid barcode molecule remains coupled to the particle in the partition; generating a polypeptide-oligonucleotide conjugate by coupling the digested polypeptide to the first nucleic acid barcode molecule in the partition; hybridizing the nucleic acid directly or indirectly to the second nucleic acid barcode molecule in the partition, thereby forming a particle complex comprising the particle, the second nucleic acid barcode molecule, and the nucleic acid; separating the polypeptide-oligonucleotide conjugate from the particle complex into a first vessel and a second vessel, respectively; generating a first barcoded nucleic acid molecule using the polypeptide-oligonucleotide conjugate, wherein the first barcoded nucleic acid molecule comprises: 1) a sequence of the first nucleic acid barcode molecule or a complement thereof, and 2) a nucleic acid sequence corresponding to an amino acid sequence of the digested polypeptide; and generating a second barcoded nucleic acid molecule using the nucleic acid and the second nucleic acid barcode molecule, wherein the second barcoded nucleic acid molecule comprises: 1) a sequence of the second nucleic acid barcode molecule or a complement thereof, and 2) a sequence of the nucleic acid or a complement thereof.

In some embodiments, the method further comprises sequencing the first barcoded nucleic acid molecule or a derivative thereof. In some embodiments, the method further comprises sequencing the second barcoded nucleic acid molecule or a derivative thereof.

Provided herein is a method comprising: providing a partition comprising: i) a digested polypeptide derived from a cell, ii) a nucleic acid derived from the cell, and iii) a particle coupled to a first nucleic acid barcode molecule and a second nucleic acid barcode molecule; selectively releasing the first nucleic acid barcode molecule from the particle in the partition, wherein the second nucleic acid barcode molecule remains coupled to the particle in the partition; generating a polypeptide-oligonucleotide conjugate by coupling the digested polypeptide to the first nucleic acid barcode molecule in the partition; hybridizing the nucleic acid directly or indirectly to the second nucleic acid barcode molecule in the partition, thereby forming a particle complex comprising the particle, the second nucleic acid barcode molecule, and the nucleic acid; separating the polypeptide-oligonucleotide conjugate from the particle complex into a first vessel and a second vessel, respectively; and generating a barcoded nucleic acid molecule using the nucleic acid and the second nucleic acid barcode molecule, wherein the barcoded nucleic acid molecule comprises: 1) a sequence of the second nucleic acid barcode molecule or a complement thereof, and 2) a sequence of the nucleic acid or a complement thereof.

In some embodiments, the method further comprises determining the sequence of the digested polypeptide and the sequence of the nucleic acid barcode molecule of the polypeptide-oligonucleotide conjugate or a derivative thereof. In some instances, determining the sequence of the digested polypeptide comprises: contacting the digested polypeptide of the polypeptide-oligonucleotide conjugate with a binding agent comprising a coding tag sequence associated with the binding agent; and extending the nucleic acid barcode molecule of the polypeptide-oligonucleotide conjugate with the coding tag sequence or a complement thereof, thereby generating an extended nucleic acid barcode molecule. In some examples, the binding agent is specific to the N-terminal amino acid residue of the digested polypeptide or a derivative thereof. In some embodiments, the method further comprises cleaving a terminal amino acid or terminal amino acid derivative from the digested polypeptide. In some embodiments, the method further comprises using additional binding agents and extending the extended nucleic acid barcode molecule with one or more additional coding tag sequences. In some instances, the extended nucleic acid barcode molecule identifies the digested polypeptide and the single cell where the digested polypeptide originated.

In some embodiments, the first nucleic acid barcode molecule is coupled to the particle via a cleavable moiety, and wherein the second nucleic acid barcode molecule is not coupled to the particle via the cleavable moiety. In some embodiments, selectively releasing the first nucleic acid barcode molecule from the particle in the partition comprises cleaving the cleavable moiety. In some embodiments, the cleavable moiety comprises a disulfide bond.

In some embodiments, the first nucleic acid barcode molecule is attached to a lysine of the digested polypeptide to form the polypeptide-oligonucleotide conjugate. In some embodiments, the first nucleic acid barcode molecule is coupled to an internal amino acid of the digested polypeptide. In some embodiments, the first nucleic acid barcode molecule is covalently coupled to the digested polypeptide. In some aspects, the coupling of the first nucleic acid barcode molecule to the digested polypeptide is copper catalyzed. In some embodiments, prior to coupling the first nucleic acid barcode molecule to the digested polypeptide, the digested polypeptide is modified to comprise an azide group. In some instances, the modification comprises contacting the digested polypeptide with a diazo transfer reagent in the presence of a copper salt and a base. In some cases, the copper salt is CuSO₄. In some cases, the base is K₂CO₃. In some cases, the diazo transfer reagent is imidazole-1-sulfonyl azide hydrochloride. In some embodiments, unreacted diazo transfer reagent is contacted with a cyclooctyne to quench the unreacted diazo transfer reagent. In some embodiments, the cyclooctyne is a dibenzocyclooctyne. In some embodiments, the quenching proceeds by way of strain-promoted alkyne-azide cycloaddition (SPAAC). In some embodiments, the coupling of the first nucleic acid barcode molecule to the digested polypeptide proceeds by way of copper-catalyzed alkyne-azide cycloaddition (CuAAC).

In some embodiments, the separating of the polypeptide-oligonucleotide conjugate from the particle complex comprises centrifugation. In some instances, the particle is magnetic, and wherein separating the polypeptide-oligonucleotide conjugate from the particle complex comprises magnetic separation.

In some embodiments, the nucleic acid comprises an endogenous nucleic acid from the cell or a derivative thereof. In some instances, the nucleic acid comprises cDNA. In some embodiments, the method comprises performing a reverse transcription reaction to generate the cDNA. In some embodiments, the reverse transcription is performed in the cell prior to providing the partition. In some embodiments, the nucleic acid comprises a fragment of genomic DNA. In some embodiments, the fragment of genomic DNA is generated using a transposase enzyme. In some embodiments, the fragment of genomic DNA is generated via tagmentation. In some embodiments, the nucleic acid is a ligation product of a probe hybridized to an analyte in the cell. In some embodiments, the analyte comprises RNA and the ligation product is a product of RNA-templated ligation. In some embodiments, the nucleic acid hybridizes directly to the second nucleic acid barcode molecule in the partition. In some embodiments, the nucleic acid hybridizes indirectly to the second nucleic acid barcode molecule in the partition via a splint molecule.

In some embodiments, the first nucleic acid barcode molecule and the second nucleic acid barcode molecule each comprise a partition-specific barcode sequence. In some embodiments, the first barcoded nucleic acid molecule comprises the partition-specific barcode sequence or a complement thereof, and wherein the second barcoded nucleic acid molecule comprises the partition-specific barcode sequence or a complement thereof. In some embodiments, the first nucleic acid barcode molecule and second nucleic acid barcode molecule comprise Unique Molecular Identifier (UMI) sequences. In some embodiments, the first nucleic acid barcode molecule and second nucleic acid barcode molecule each comprise one or more functional sequences.

In some embodiments, the partition is a droplet among a plurality of droplets. In some embodiments, the partition is a well among a plurality of wells. In some embodiments, the particle is a bead. In some embodiments, the method comprises lysing the cell in the partition.

In some embodiments, the digested polypeptide is a digestion product of a protein from the cell. In some instances, the digested polypeptide is from a cell surface protein. In some embodiments, the digested polypeptide is from an intracellular protein. In some embodiments, the digested polypeptide is from an extracellular protein.

### BRIEF DESCRIPTION OF THE DRAWINGS

The drawings illustrate certain features and advantages of this disclosure. These embodiments are not intended to limit the scope of the appended claims in any manner.

FIG. 1A shows an example workflow for generating a nucleic acid barcode molecule conjugated to a polypeptide to provide a polypeptide-oligonucleotide conjugate for downstream analysis.

FIG. 1B shows an example of a microfluidic channel structure for partitioning.

FIG. 2 shows an example of a microfluidic channel structure for the controlled partitioning of beads into discrete droplets.

FIG. 3 shows an example of a microfluidic channel structure for delivering barcode carrying beads to droplets.

FIG. 4 schematically illustrates an example microwell array.

FIG. 5 illustrates amplification of a sequence in a nucleic acid sequence of a polypeptide-oligonucleotide conjugate for detection.

FIG. 6 is a schematic illustrating an example workflow for parallel analysis of polypeptides and nucleic acids from single cells.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Provided herein are methods, compositions, and systems for analysis of polypeptides (e.g., peptides and proteins) at the single cell level. Different cells comprise dynamic collections of protein molecules depending on a number of factors including the cellular environment and physiological state (e.g., conditions such as disease) with added diversity introduced by post-translational modifications (PTMs). Antibody based methods for protein analysis are limited, indirect, require antibodies to proteins of interest, and are not amenable to analysis of the whole proteome expressed in each single cell. The technology provided herein uses nucleic acid barcode molecules to label polypeptides (e.g., peptides and proteins) present in a single cell to provide information regarding the origin of a polypeptide.

In some embodiments, provided are nucleic acid barcode molecules in a partition for labeling the polypeptides. In some aspects, polypeptides associated with a nucleic acid barcode molecule indicating the cell of origin are sequenced. In some instances, the amino acid sequence of a polypeptide is determined for at least a portion of the polypeptide sufficient to determine the identity of the polypeptide (e.g., the protein identity). In some aspects, provided herein are methods for polypeptide sequencing of proteins or portions thereof expressed by the single cell. In some aspects, polypeptides associated with the same nucleic acid barcode molecule sequence that was uniquely present in a specific partition are identified as arising from the same single cell that was provided to the same partition.

In some embodiments, a plurality of proteins (e.g., in single cells) is treated with an enzyme to digest the proteins into fragments. In some embodiments, a plurality of proteins (e.g., in single cells) is treated with a chemical agent to digest the proteins into fragments. In some instances, the enzyme is a digestion enzyme. In some instances, the enzyme is a protease. In some instances, the enzyme is trypsin. In some instances, the proteins are cleaved at a specific amino acid residue. In some embodiments, a plurality of polypeptides is generated from digesting a plurality of proteins. In some instances, a digested polypeptide is a treated protein (e.g., a treated polypeptide). In some instances, a digested polypeptide is a digestion product of a protein molecule. In some instances, a digested polypeptide is a digestion product of a protein originating from a cell. In some instances, a digested protein generates a plurality of digested polypeptides (e.g., in a single cell). In some instances, a digested polypeptide comprises more than one amino acid residue. In some instances, following enzymatic or chemical treatment, a plurality of digested polypeptides comprises fragments that are each from about 10 amino acids to about 100 amino acids.

In some embodiments, provided herein is a workflow comprising: providing an emulsion comprising a droplet population, wherein a droplet of the droplet population comprises i) a plurality of digested polypeptides from a single cell, and ii) a nucleic acid barcode molecule coupled to a particle; subjecting the emulsion to conditions sufficient to transfer a conjugation reagent to the droplet population (e.g., via micelles); and using a reducing agent to release the nucleic acid barcode molecule coupled to the particle; thereby generating a polypeptide-oligonucleotide conjugate by coupling a digested polypeptide of the plurality of digested polypeptides to the nucleic acid barcode molecule. In some embodiments, the partition comprises a nucleic acid derived from the same cell as the digested polypeptides and the particle is coupled to an additional nucleic acid barcode molecule. In some aspects, the nucleic acid barcode molecule is selectively released from the particle in the partition for generating a polypeptide-oligonucleotide conjugate, wherein the additional nucleic acid barcode molecule remains coupled to the particle in the partition. In some aspects, the nucleic acid (e.g., a generated cDNA in the partition) directly or indirectly hybridizes to the additional nucleic acid barcode molecule in the partition, thereby forming a particle complex comprising the particle, the additional nucleic acid barcode molecule, and the nucleic acid. In some aspects, the polypeptide-oligonucleotide conjugate is separated from the particle complex into a first vessel and a second vessel, respectively, for downstream processing and analysis of the digested polypeptide and the nucleic acid from the same cell. In some aspects, a barcoded nucleic acid molecule using the nucleic acid and the additional nucleic acid barcode molecule is generated. In some aspects, to determine the sequence of the digested polypeptide, the digested polypeptide of the polypeptide-oligonucleotide conjugate is contacted with a binding agent comprising a coding tag sequence associated with the binding agent; the nucleic acid barcode molecule of the polypeptide-oligonucleotide conjugate is extended (e.g., by ligation or polymerase extension) with the coding tag sequence or a complement thereof, thereby generating an extended nucleic acid barcode molecule. In some embodiments, the resulting extended nucleic acid barcode molecule is sequenced to determine the sequence of the digested polypeptide or a portion thereof and the generated barcoded nucleic acid molecule using the nucleic acid and the additional nucleic acid barcode molecule is also sequenced.

In some embodiments, provided herein is a workflow comprising: providing a plurality of digested polypeptides from a single cell in a partition (e.g., a well), wherein the partition comprises a plurality of nucleic acid barcode molecules unique to the partition; and providing a reducing agent and a conjugation reagent to the partition to generate a plurality of polypeptide-oligonucleotide conjugates by coupling the plurality of digested polypeptides to the plurality of nucleic acid barcode molecules. In some embodiments, the partition comprises a nucleic acid derived from the same cell as the digested polypeptides and an additional nucleic acid barcode molecule is provided in the partition. In some aspects, the nucleic acid barcode molecule is used for generating a polypeptide-oligonucleotide conjugate, wherein the additional nucleic acid barcode molecule is used to barcoding a different type of analyte (e.g., a cDNA from the same cell). In some aspects, the nucleic acid (e.g., a generated cDNA in the partition) directly or indirectly hybridizes to the additional nucleic acid barcode molecule in the partition, thereby forming a particle complex comprising the particle, the additional nucleic acid barcode molecule, and the nucleic acid. In some aspects, the polypeptide-oligonucleotide conjugate is collected for downstream processing and analysis of the digested polypeptide and the nucleic acid from the same cell. In some aspects, a barcoded nucleic acid molecule using the nucleic acid and the additional nucleic acid barcode molecule is generated. In some aspects, to determine the sequence of the digested polypeptide, the digested polypeptide of the polypeptide-oligonucleotide conjugate is contacted with a binding agent comprising a coding tag sequence associated with the binding agent; the nucleic acid barcode molecule of the polypeptide-oligonucleotide conjugate is extended (e.g., by ligation or polymerase extension) with the coding tag sequence or a complement thereof, thereby generating an extended nucleic acid barcode molecule. In some embodiments, the resulting extended nucleic acid barcode molecule is sequenced to determine the sequence of the digested polypeptide or a portion thereof and the generated barcoded nucleic acid molecule using the nucleic acid and the additional nucleic acid barcode molecule is also sequenced.

In some embodiments, provided herein is a workflow comprising: providing an emulsion comprising a droplet population, wherein a droplet of the droplet population comprises i) a plurality of digested polypeptides from a single cell, and ii) a nucleic acid barcode molecule coupled to a particle; subjecting the emulsion to conditions sufficient to transfer a conjugation reagent to the droplet population via micelles; and using a reducing agent to release the nucleic acid barcode molecule coupled to the particle; thereby generating a polypeptide-oligonucleotide conjugate by coupling a digested polypeptide of the plurality of digested polypeptides to the nucleic acid barcode molecule. In some aspects, the generated plurality of polypeptide-oligonucleotide conjugates are dispersed on a substrate; and a plurality of signals associated with (i) the digested polypeptide, and (ii) the nucleic acid barcode molecule, are detected at a location on the substrate. In some aspects, a sequence of the nucleic acid barcode molecule is amplified (e.g., by RCA). In some embodiments, the nucleic acid barcode molecule of the polypeptide-oligonucleotide conjugate on the substrate is contacted with a circularizable probe that is gap filled and ligated upon binding to the nucleic acid barcode molecule. In some aspects, the plurality of signals are generated by binding detectably labeled probes to the RCA product. In some aspects, two or more signals are sequentially detected at a location of the polypeptide-oligonucleotide conjugate from two or more detectably labeled probes that sequentially bind to the RCA product. In some aspects, to determine the sequence of the digested polypeptide, the digested polypeptide of the polypeptide-oligonucleotide conjugate is contacted with a binding agent comprising a detectable label.

In some embodiments, provided herein is a workflow comprising: providing a plurality of digested polypeptides from a single cell in a partition (e.g., a well), wherein the partition comprises a plurality of nucleic acid barcode molecules unique to the partition; and providing a reducing agent and a conjugation reagent to the partition to generate a plurality of polypeptide-oligonucleotide conjugates by coupling the plurality of digested polypeptides to the plurality of nucleic acid barcode molecules. In some aspects, the generated plurality of polypeptide-oligonucleotide conjugates are dispersed on a substrate; and a plurality of signals associated with (i) the digested polypeptide, and (ii) the nucleic acid barcode molecule, are detected at a location on the substrate. In some aspects, a sequence of the nucleic acid barcode molecule is amplified (e.g., by RCA). In some embodiments, the nucleic acid barcode molecule of the polypeptide-oligonucleotide conjugate on the substrate is contacted with a circularizable probe that is gap filled and ligated upon binding to the nucleic acid barcode molecule. In some aspects, the plurality of signals are generated by binding detectably labeled probes to the RCA product. In some aspects, to determine the sequence of the digested polypeptide, the digested polypeptide of the polypeptide-oligonucleotide conjugate is contacted with a binding agent comprising a detectable label.

### II. Partitioning Cells

In some aspects, to achieve single cell proteomic analysis, intact cells are compartmentalized into discrete compartments or partitions along with nucleic acid barcode molecules. In some embodiments, the nucleic acid barcode molecules are coupled to a particle (e.g., a bead). It is important for the barcoding process that contents of a single cell are maintained in a partition separated from contents of a different cell, however, introducing a reagent in a droplet while maintaining the contents of the droplet remains challenging. In some instances, a reagent is introduced to a formed droplet (e.g., a droplet comprising contents from a single cell). In some embodiments, the reagent is for cleaving or formation of a bond, reduction, oxidation, hydrolysis, etc. Accordingly, recognized herein is a need for improved systems and methods for processing and preparing polypeptides for nucleic acid barcoding. In some instances, systems and methods described herein are useful for introducing one or more reagents to a droplet for preparing a sample for polypeptide analysis.

In some instances, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions. In some instances, the partition is a droplet in an emulsion or a well. In some instances, a partition comprises one or more other partitions.

In some instances, the partition comprises one or more particles. A partition may include one or more types of particles. For example, a partition of the present disclosure may comprise one or more biological particles and/or macromolecular constituents thereof. In some instances, the partition comprises one or more beads. In some instances, the partition comprises one or more gel beads. In some instances, the partition comprises one or more cell beads. In some instances, the partition comprises a single gel bead, a single cell bead, or both a single cell bead and single gel bead. In some instances, the partition comprises one or more reagents. Alternatively, a partition may be unoccupied. For example, a partition may not comprise a bead.

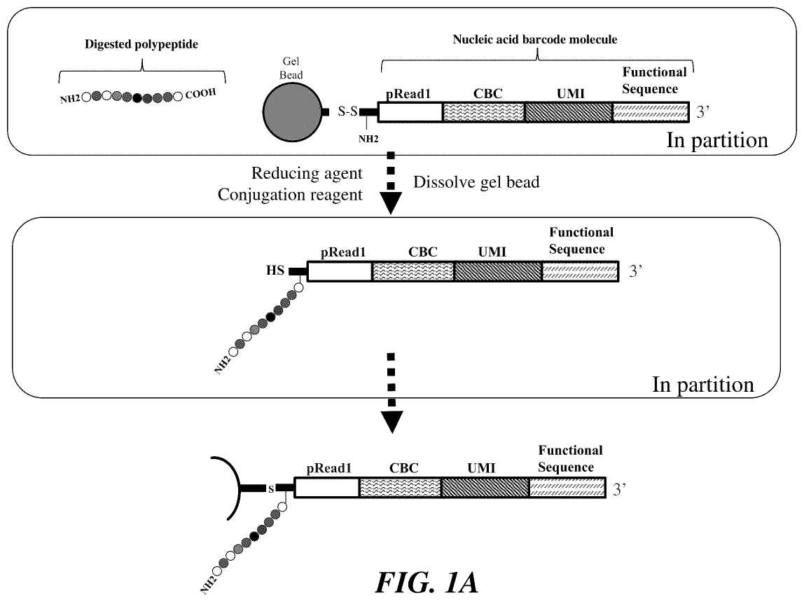

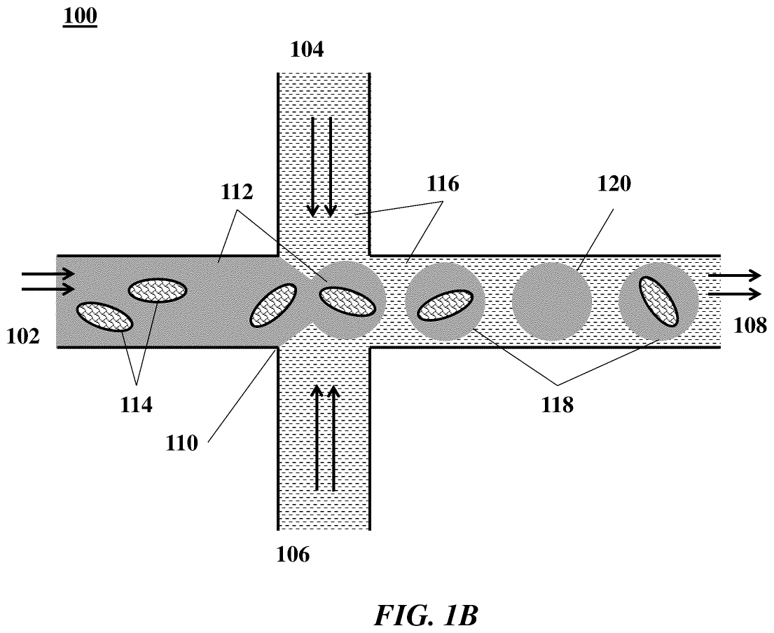

Provided herein is a method comprising providing an emulsion comprising a droplet population, wherein a droplet of the droplet population comprises i) a plurality of digested polypeptides from a single cell, and ii) a nucleic acid barcode molecule coupled to a particle; subjecting the emulsion to conditions sufficient to transfer a conjugation reagent to the droplet population (e.g., via micelles); and using a reducing agent to release the nucleic acid barcode molecule coupled to the particle; thereby generating a polypeptide-oligonucleotide conjugate by coupling a digested polypeptide of the plurality of digested polypeptides to the nucleic acid barcode molecule. In some instances, nucleic acid barcode molecules are introduced into the droplets previous to, subsequent to, or concurrently with droplet generation, such as via a bead, as described elsewhere herein. FIG. 1A shows an example workflow for generating a nucleic acid barcode molecule conjugated to a polypeptide to provide a polypeptide-oligonucleotide conjugate.

Provided herein is a method comprising providing a partition (e.g., a well), where a single well comprises i) a plurality of digested polypeptides from a single cell, and ii) a nucleic acid barcode molecule; providing the partition with a conjugation reagent; and generating a polypeptide-oligonucleotide conjugate by coupling a digested polypeptide of the plurality of digested polypeptides to the nucleic acid barcode molecule. In some instances, nucleic acid barcode molecules are introduced into the partition previous to, subsequent to, or concurrently the digested polypeptides to the partition.

### A. Nucleic Acid Barcode Molecules

In some embodiments, the nucleic acid barcode molecule comprises one or more barcode sequences. In some embodiments, a plurality of nucleic acid barcode molecules are coupled to a particle (e.g., a bead). The one or more barcode sequences may include sequences that are the same for all or a portion of the nucleic acid barcode molecules coupled to a given bead. In some instances, the nucleic acid barcode molecules are incorporated into the bead. In some embodiments, a nucleic acid barcode molecule comprises one or more functional sequences. In some cases, the nucleic acid barcode molecule comprises one or more functional sequences, for example, for attachment to a sequencing flow cell, such as, for example, a P5 sequence (or a portion thereof) for Illumina® sequencing. In some cases, the nucleic acid barcode molecule or derivative thereof comprises another functional sequence, such as, for example, a P7 sequence (or a portion thereof) for attachment to a sequencing flow cell for Illumina sequencing. In some cases, the nucleic acid barcode molecule comprises an R1 primer sequence for Illumina sequencing. In some cases, the nucleic acid barcode molecule comprises an R2 primer sequence for Illumina sequencing. In some cases, a functional sequence comprises a partial sequence, such as a partial barcode sequence, partial anchoring sequence, partial sequencing primer sequence (e.g., partial R1 sequence, partial R2 sequence, etc.), a partial sequence configured to attach to the flow cell of a sequencer (e.g., partial P5 sequence, partial P7 sequence, etc.), or a partial sequence of any other type of sequence described elsewhere herein. A partial sequence may contain a contiguous or continuous portion or segment, but not all, of a full sequence, for example. In some cases, a downstream procedure may extend the partial sequence, or derivative thereof, to achieve a full sequence of the partial sequence, or derivative thereof. Examples of such nucleic acid barcode molecules and uses thereof, as may be used with compositions, devices, methods and systems of the present disclosure, are provided in U.S. Patent Pub. Nos. 2014/0378345 and 2015/0376609, each of which is entirely incorporated herein by reference.

In some embodiments, a nucleic acid barcode molecule is coupled to a particle (e.g., a bead) by a releasable linkage, such as, for example, a disulfide linker as shown in FIG. 1A. In some instances, the nucleic acid barcode molecule comprises a barcode (e.g., a cell barcode (CBC) associated with the originating cell and/or partition). In some embodiments, a nucleic acid barcode molecule comprises a functional sequence for subsequent processing. In some embodiments, a functional sequence comprises one or more of a sequencer specific flow cell attachment sequence (e.g., a P5 sequence for Illumina® sequencing systems) and a sequencing primer sequence (e.g., a R1 primer for Illumina® sequencing systems), or partial sequence(s) thereof. In some cases, the barcode sequence is bead-specific such that the barcode sequence (CBC) is common to all nucleic acid barcode molecules coupled to the same bead. Alternatively, or in addition, the barcode sequence is partition-specific such that the barcode sequence is common to all nucleic acid barcode molecules coupled to one or more beads that are partitioned into the same partition.

In some cases, the nucleic acid barcode molecule comprises a unique molecular identifier (UMI). In some cases, the unique molecular identifying sequence is about 5 to about 8 nucleotides. Alternatively, the unique molecular identifying sequence is less than about 5 or more than about 8 nucleotides. In some cases, the unique molecular identifying sequence is a unique sequence that varies across individual nucleic acid barcode molecules coupled to a single particle (e.g., bead). In some cases, the unique molecular identifying sequence is a random sequence (e.g., such as a random N-mer sequence). For example, the UMI may provide a unique identifier of the coupled digested polypeptide.

In some embodiments, a barcode sequence (e.g., cell barcode sequence) of a nucleic acid barcode molecule is from about 6 to about 20 or more nucleotides within the sequence of the nucleic acid barcode molecule. The barcode sequences can include from about 6 to about 20, 30, 40, 50, 60, 70, 80, 90, 100 or more nucleotides. In some cases, the length of a barcode sequence is about 6, 7, 8, 9, 10, 11, 12, 13, 14, 15, 16, 17, 18, 19, 20 nucleotides or longer. In some cases, the length of a barcode sequence is at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. In some instances, these nucleotides are completely contiguous, i.e., in a single stretch of adjacent nucleotides, or in some cases, they are separated into two or more separate subsequences that are separated by 1 or more nucleotides. In some cases, separated barcode subsequences are from about 4 to about 16 nucleotides in length. In some cases, the barcode subsequence is about 4, 5, 6, 7, 8, 9, 10, 11, 12, 13, 14, 15, 16 nucleotides or longer. In some cases, the barcode subsequence is at least about 4, 5, 6, 7, 8, 9, 10, 11, 12, 13, 14, 15, 16 nucleotides or longer. In some cases, the barcode subsequence is at most about 4, 5, 6, 7, 8, 9, 10, 11, 12, 13, 14, 15, 16 nucleotides or shorter.

In some embodiments, a biological particle (e.g., cell, DNA, RNA, etc.) is co-partitioned along with a nucleic acid barcode molecule coupled to a particle and processed to generate a polypeptide-oligonucleotide conjugate by coupling a digested polypeptide to the nucleic acid barcode molecule. The operations described herein may be performed at any useful or convenient step. For instance, the beads comprising nucleic acid barcode molecules may be introduced into a partition (e.g., well or droplet) prior to, during, or following introduction of a sample into the partition. In some embodiments, the cell is treated with reagents to digest proteins and the digested polypeptides are subjected to barcoding. In some instances, barcoding of digested polypeptides follow release of the nucleic acid barcode molecules into the partition. In some instances, the beads from various partitions may be collected, pooled, and subjected to further processing. In some instances, the processing occurs in the partition. For example, conditions sufficient for barcoding is provided for coupling a digested polypeptide of a plurality of digested polypeptides from a single cell to the nucleic acid barcode molecule.

In some embodiments, beads are provided that include large numbers of the above-described nucleic acid barcode molecules releasably attached to the beads, where all or at least a subset of the nucleic acid barcode molecules attached to a particular bead will include a common nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid barcode molecules into the partitions, as they are capable of carrying large numbers of nucleic acid barcode molecules, and may be configured to release nucleic acid barcode molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. In some cases, the population of beads provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences.

In some embodiments, nucleic acid barcode molecules of a given bead comprise identical (or common) barcode sequences, different barcode sequences, or a combination of both. In some embodiments, nucleic acid barcode molecules of a given set comprise identical barcode sequences. The identical barcode sequences can be different from barcode sequences of nucleic acid barcode molecules of another set.

In some embodiments, when the population of beads is partitioned, the resulting population of partitions includes a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid barcode molecules, at least about 5,000 nucleic acid barcode molecules, at least about 10,000 nucleic acid barcode molecules, at least about 50,000 nucleic acid barcode molecules, at least about 100,000 nucleic acid barcode molecules, at least about 500,000 nucleic acids, at least about 1,000,000 nucleic acid barcode molecules, at least about 5,000,000 nucleic acid barcode molecules, at least about 10,000,000 nucleic acid barcode molecules, at least about 50,000,000 nucleic acid barcode molecules, at least about 100,000,000 nucleic acid barcode molecules, at least about 250,000,000 nucleic acid barcode molecules and in some cases at least about 1 billion nucleic acid barcode molecules.

In some embodiments, the resulting population of partitions provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences. Additionally, each partition of the population can include between about 1,000 to about 10,000 nucleic acid barcode molecules, about 5,000 to about 50,000 nucleic acid barcode molecules, about 10,000 to about 100,000 nucleic acid barcode molecules, about 50,000 to about 1,000,000 nucleic acid barcode molecules, about 100,000 to about 10,000,000 nucleic acid barcode molecules, about 1,000,000 to about 1 billion nucleic acid barcode molecules.

In some embodiments, multiple different barcodes are incorporated within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

In some embodiments, the nucleic acid barcode molecules are released (e.g., in a partition), as described elsewhere herein. A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

In some instances, the nucleic acid barcode molecule is derivatized with a reactive groups (e.g., compatible for conjugation as described in Section II.E). In some instances, the nucleic acid barcode molecule is derivatized with a reactive group that is compatible for conjugation using click chemistry. In some instances, the nucleic acid barcode molecule comprises an alkyne. In some instances, the nucleic acid barcode molecule comprises: 5′ acrydite—disulfide—internal alkyne.

### B. Partitions

Provided herein are methods and systems for generating one or more partitions such as droplets or wells. In some embodiments, the droplets comprise a plurality of droplets in an emulsion. In some examples, the droplets comprise droplets in a colloid. In some cases, the emulsion comprises a microemulsion or a nanoemulsion. In some examples, the droplets are generated with aid of a microfluidic device and/or by subjecting a mixture of immiscible phases to agitation (e.g., in a container). In some cases, a combination of the mentioned methods may be used for droplet and/or emulsion formation.

In some embodiments, the partitions comprise small volumes, for example, less than about 10 microliters (μL), 5 μL, 1 μL, 10 nanoliters (nL), 5 nL, 1 nL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less. For example, in the case of droplet-based partitions, the droplets may have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with beads, it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

In some embodiments, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions.

In some embodiments, partitions are formed by creating an emulsion by mixing and/or agitating immiscible phases. Mixing or agitation may comprise various agitation techniques, such as vortexing, pipetting, tube flicking, or other agitation techniques. In some cases, mixing or agitation may be performed without using a microfluidic device. In some examples, the droplets may be formed by exposing a mixture to ultrasound or sonication.

In some embodiments, microfluidic devices or platforms comprising microfluidic channel networks (e.g., on a chip) are utilized to generate partitions such as droplets and/or emulsions as described herein. Methods and systems for generating partitions such as droplets, methods of encapsulating biological particles in partitions, methods of increasing the throughput of droplet generation, and various geometries, architectures, and configurations of microfluidic devices and channels are described in U.S. Patent Application Publication Nos. 2019/0367997 and 2019/0064173, each of which is entirely incorporated herein by reference for all purposes.

In some examples, individual particles are partitioned to discrete partitions by introducing a flowing stream of particles in an aqueous fluid into a flowing stream or reservoir of a non-aqueous fluid, such that droplets may be generated at the junction of the two streams/reservoir, such as at the junction of a microfluidic device provided elsewhere herein.

Provided herein are methods for generating partitions and/or encapsulating particles, such as biological particles, in some cases, individual biological particles such as single cells. In some examples, reagents may be encapsulated and/or partitioned (e.g., co-partitioned with biological particles) in the partitions. Various mechanisms may be employed in the partitioning of individual particles. An example may comprise porous membranes through which aqueous mixtures of cells may be extruded into fluids (e.g., non-aqueous fluids).

In some embodiments, partitions are flowable within fluid streams. In some embodiments, partitions comprise micro-vesicles that have an outer barrier surrounding an inner fluid center or core. In some cases, the partitions comprise a porous matrix that is capable of entraining and/or retaining materials within its matrix. In some embodiments, partitions are droplets of a first phase within a second phase, wherein the first and second phases are immiscible. For example, the partitions can be droplets of aqueous fluid within a non-aqueous continuous phase (e.g., oil phase). In another example, the partitions are droplets of a non-aqueous fluid within an aqueous phase. In some examples, the partitions are provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

In some embodiments, fluid properties (e.g., fluid flow rates, fluid viscosities, etc.), particle properties (e.g., volume fraction, particle size, particle concentration, etc.), microfluidic architectures (e.g., channel geometry, etc.), and other parameters may be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions may contain less than one biological particle per partition to ensure that those partitions that are occupied are primarily singly occupied. In some cases, partitions among a plurality of partitions may contain at most one biological particle (e.g., bead, DNA, cell or cellular material). In some embodiments, the various parameters (e.g., fluid properties, particle properties, microfluidic architectures, etc.) may be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

FIG. 1B shows an example of a microfluidic channel structure 100 for partitioning individual biological particles. The channel structure 100 can include channel segments 102, 104, 106 and 108 communicating at a channel junction 110. In operation, a first aqueous fluid 112 that includes suspended biological particles (or cells) 114 may be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual biological particle 114 (such as droplets 118). A discrete droplet generated may include more than one individual biological particle 114 (not shown in FIG. 1B). A discrete droplet may contain no biological particle 114 (such as droplet 120). Each discrete partition may maintain separation of its own contents (e.g., individual biological particle 114) from the contents of other partitions. The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein may be coupled to any of a variety of different fluid sources or receiving components, including reservoirs, tubing, manifolds, or fluidic components of other systems. As will be appreciated, the microfluidic channel structure 100 may have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid may be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 118, containing one or more biological particles 114, and (2) unoccupied droplets 120, not containing any biological particles 114. Occupied droplets 118 may comprise singly occupied droplets (having one biological particle) and multiply occupied droplets (having more than one biological particle). As described elsewhere herein, in some cases, the majority of occupied partitions can include no more than one biological particle per occupied partition and some of the generated partitions can be unoccupied (of any biological particle). In some cases, though, some of the occupied partitions may include more than one biological particle. In some cases, the partitioning process may be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it may be desirable to minimize the creation of excessive numbers of empty partitions, such as to reduce costs and/or increase efficiency. While this minimization may be achieved by providing a sufficient number of biological particles (e.g., biological particles 114) at the partitioning junction 110, such as to ensure that at least one biological particle is encapsulated in a partition, the Poissonian distribution may expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, flows can be controlled so as to present a non-Poissonian distribution of single-occupied partitions while providing lower levels of unoccupied partitions (e.g., no more than about 50%, about 25%, or about 10% unoccupied). The above noted ranges of unoccupied partitions can be achieved while still providing any of the single occupancy rates described above. As will be appreciated, the above-described occupancy rates are also applicable to partitions that include both biological particles and additional reagents, such as beads (e.g., gel beads) carrying nucleic acid barcode molecules (e.g., oligonucleotides).

In some examples, a partition of the plurality of partitions comprises a single biological particle (e.g., a single cell or a single nucleus of a cell). In some examples, a partition of the plurality of partitions comprises multiple biological particles. Such partitions may be referred to as multiply occupied partitions, and may comprise, for example, two, three, four or more cells and/or beads (e.g., beads) comprising nucleic acid barcode molecules within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher. Microfluidic systems for partitioning are further described in U.S. Patent Application Pub. No. 2015/0376609, which is hereby incorporated by reference in its entirety.

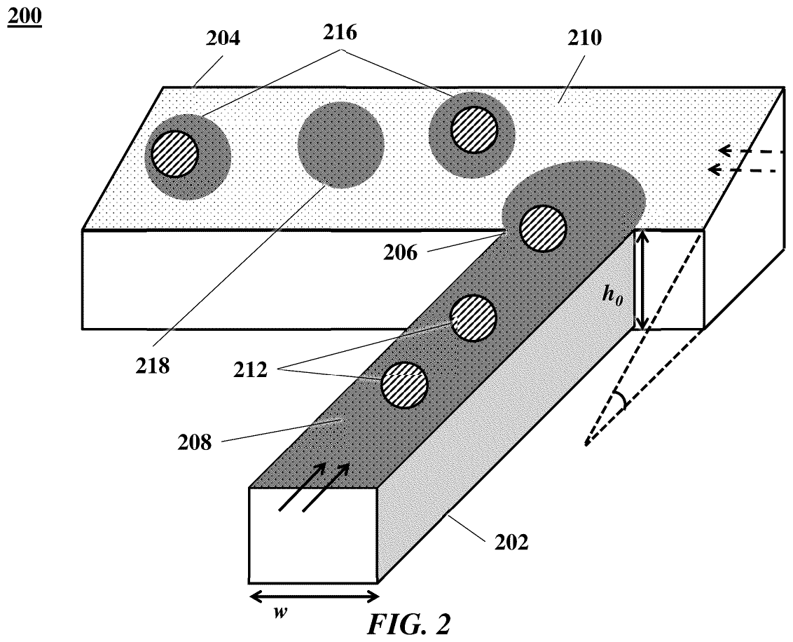

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size. FIG. 2 shows an example of a microfluidic channel structure for the controlled partitioning of beads into discrete droplets. A channel structure 200 can include a channel segment 202 communicating at a channel junction 206 (or intersection) with a reservoir 204. The reservoir 204 can be a chamber. Any reference to “reservoir,” as used herein, can also refer to a “chamber.” In operation, an aqueous fluid 208 that includes suspended beads 212 may be transported along the channel segment 202 into the junction 206 to meet a second fluid 210 that is immiscible with the aqueous fluid 208 in the reservoir 204 to create droplets 216, 218 of the aqueous fluid 208 flowing into the reservoir 204. At the junction 206 where the aqueous fluid 208 and the second fluid 210 meet, droplets can form based on factors such as the hydrodynamic forces at the junction 206, flow rates of the two fluids 208, 210, fluid properties, and certain geometric parameters (e.g., w, h₀, α, etc.) of the channel structure 200. A plurality of droplets can be collected in the reservoir 204 by continuously injecting the aqueous fluid 208 from the channel segment 202 through the junction 206.

In some instances, the aqueous fluid 208 can have a substantially uniform concentration or frequency of beads 212. The beads 212 can be introduced into the channel segment 202 from a separate channel (not shown in FIG. 2). The frequency of beads 212 in the channel segment 202 may be controlled by controlling the frequency in which the beads 212 are introduced into the channel segment 202 and/or the relative flow rates of the fluids in the channel segment 202 and the separate channel. In some instances, the beads are introduced into the channel segment 202 from a plurality of different channels, and the frequency controlled accordingly. In some instances, the aqueous fluid 208 in the channel segment 202 can comprise biological particles. In some instances, the aqueous fluid 208 can have a substantially uniform concentration or frequency of biological particles. As with the beads, the biological particles can be introduced into the channel segment 202 from a separate channel. The frequency or concentration of the biological particles in the aqueous fluid 208 in the channel segment 202 may be controlled by controlling the frequency in which the biological particles are introduced into the channel segment 202 and/or the relative flow rates of the fluids in the channel segment 202 and the separate channel. In some instances, the biological particles can be introduced into the channel segment 202 from a plurality of different channels, and the frequency controlled accordingly. In some instances, a first separate channel can introduce beads and a second separate channel can introduce biological particles into the channel segment 202. The first separate channel introducing the beads may be upstream or downstream of the second separate channel introducing the biological particles. The second fluid 210 can comprise an oil, such as a fluorinated oil, that includes a fluorosurfactant for stabilizing the resulting droplets, for example, inhibiting subsequent coalescence of the resulting droplets. In some instances, the second fluid 210 may not be subjected to and/or directed to any flow in or out of the reservoir 204. For example, the second fluid 210 may be substantially stationary in the reservoir 204. In some instances, the second fluid 210 may be subjected to flow within the reservoir 204, but not in or out of the reservoir 204, such as via application of pressure to the reservoir 204 and/or as affected by the incoming flow of the aqueous fluid 208 at the junction 206. Alternatively, the second fluid 210 may be subjected and/or directed to flow in or out of the reservoir 204. For example, the reservoir 204 can be a channel directing the second fluid 210 from upstream to downstream, transporting the generated droplets. Systems and methods for controlled partitioning are described further in U.S. Patent Application Publication No. 2019/0060890 and 2020/0290048, which are hereby incorporated by reference in their entirety.

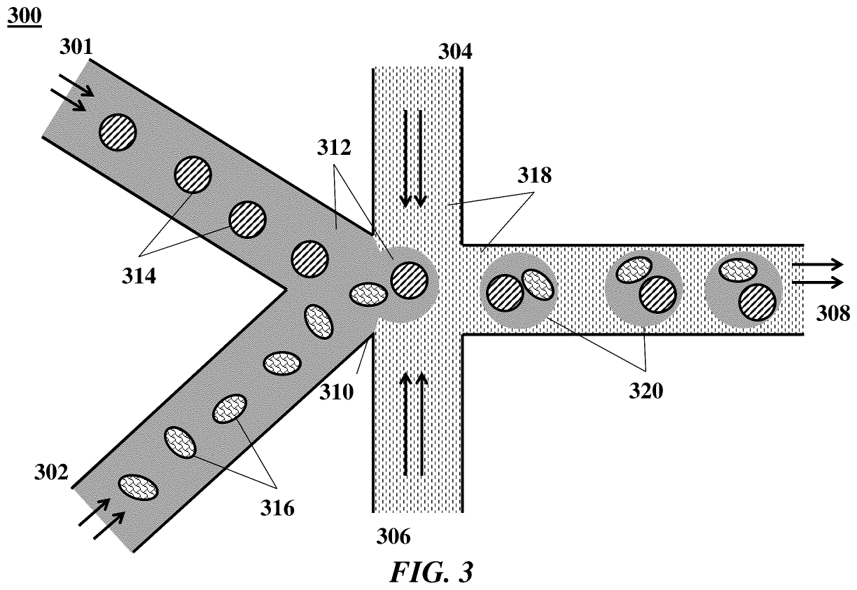

FIG. 3 shows an example of a microfluidic channel structure 300 for delivering barcode carrying beads to droplets. The channel structure 300 can include channel segments 301, 302, 304, 306 and 308 communicating at a channel junction 310. In operation, the channel segment 301 may transport an aqueous fluid 312 that includes a plurality of beads 314 (e.g., with nucleic acid barcode molecules or molecular tags) along the channel segment 301 into junction 310. The plurality of beads 314 may be sourced from a suspension of beads. For example, the channel segment 301 may be connected to a reservoir comprising an aqueous suspension of beads 314. The channel segment 302 may transport the aqueous fluid 312 that includes a plurality of biological particles 316 along the channel segment 302 into junction 310. The plurality of biological particles 316 may be sourced from a suspension of biological particles. For example, the channel segment 302 may be connected to a reservoir comprising an aqueous suspension of biological particles 316. In some instances, the aqueous fluid 312 in either the first channel segment 301 or the second channel segment 302, or in both segments, can include one or more reagents, as further described below. A second fluid 318 that is immiscible with the aqueous fluid 312 (e.g., oil) can be delivered to the junction 310 from each of channel segments 304 and 306. Upon meeting of the aqueous fluid 312 from each of channel segments 301 and 302 and the second fluid 318 from each of channel segments 304 and 306 at the channel junction 310, the aqueous fluid 312 can be partitioned as discrete droplets 320 in the second fluid 318 and flow away from the junction 310 along channel segment 308. The channel segment 308 may deliver the discrete droplets to an outlet reservoir fluidly coupled to the channel segment 308, where they may be harvested. As an alternative, the channel segments 301 and 302 may meet at another junction upstream of the junction 310. At such junction, beads and biological particles may form a mixture that is directed along another channel to the junction 310 to yield droplets 320. The mixture may provide the beads and biological particles in an alternating fashion, such that, for example, a droplet comprises a single bead and a single biological particle.

In some embodiments, one or more processes are performed in a partition, which is a well. In some instances, the well is a well of a plurality of wells of a substrate, such as a microwell of a microwell array or plate, or the well is a microwell or microchamber of a device (e.g., microfluidic device) comprising a substrate. In some instances, the well is a well of a well array or plate, or the well is a well or chamber of a device (e.g., fluidic device). In some embodiments, a well of a fluidic device is fluidically connected to another well of the fluidic device. Accordingly, the wells or microwells may assume an “open” configuration, in which the wells or microwells are exposed to the environment (e.g., contain an open surface) and are accessible on one planar face of the substrate, or the wells or microwells may assume a “closed” or “sealed” configuration, in which the microwells are not accessible on a planar face of the substrate. In some instances, the wells or microwells may be configured to toggle between “open” and “closed” configurations. For instance, an “open” microwell or set of microwells may be “closed” or “sealed” using a membrane (e.g., semi-permeable membrane), an oil (e.g., fluorinated oil to cover an aqueous solution), or a lid, as described elsewhere herein.

In some instances, the well has a volume of less than 1 milliliter (mL). For instance, the well may be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well may be configured to hold a volume of about 1000 μL, about 100 μL, about 10 μL, about 1 μL, about 100 nL, about 10 nL, about 1 nL, about 100 pL, about 10 pL, etc. The well may be configured to hold a volume of at least 10 pL, at least 100 pL, at least 1 nL, at least 10 nL, at least 100 nL, at least 1 μL, at least 10 μL, at least 100 μL, at least 1000 μL, or more. The well may be configured to hold a volume in a range of volumes listed herein, for example, from about 5 nL to about 20 nL, from about 1 nL to about 100 nL, from about 500 pL to about 100 μL, etc. The well may be of a plurality of wells that have varying volumes and may be configured to hold a volume appropriate to accommodate any of the partition volumes described herein.

In some instances, a microwell array or plate comprises a single variety of microwells. In some instances, a microwell array or plate comprises a variety of microwells. For instance, the microwell array or plate may comprise one or more types of microwells within a single microwell array or plate. The types of microwells may have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate may comprise any number of different types of microwells. For example, the microwell array or plate may comprise 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well may have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate comprises different types of microwells that are located adjacent to one another within the array or plate. For instance, a microwell with one set of dimensions may be located adjacent to and in contact with another microwell with a different set of dimensions. Similarly, microwells of different geometries may be placed adjacent to or in contact with one another. The adjacent microwells may be configured to hold different articles; for example, one microwell may be used to contain a cell, cell bead, or other sample while the adjacent microwell may be used to contain a droplet, bead, or other reagent. In some cases, the adjacent microwells may be configured to merge the contents held within, e.g., upon application of a stimulus, or spontaneously, upon contact of the articles in each microwell.

As is described elsewhere herein, a plurality of partitions may be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells may comprise both unoccupied wells (e.g., empty wells) and occupied wells.

In some instances, the well comprises any of the reagents described herein, or combinations thereof. These reagents may include, for example, nucleic acid barcode molecules, enzymes, adapters, and combinations thereof. The reagents may be physically separated from polypeptides from a cell placed in the well. This physical separation may be accomplished by containing the reagents within, or coupling to, a bead that is placed within a well. The physical separation may also be accomplished by dispensing the reagents in the well and overlaying the reagents with a layer that is, for example, dissolvable, meltable, or permeable prior to introducing the polynucleotide sample into the well. This layer may be, for example, an oil, wax, membrane (e.g., semi-permeable membrane), or the like. The well may be sealed at any point, for example, after addition of the bead, after addition of the reagents, or after addition of either of these components. The sealing of the well may be useful for a variety of purposes, including preventing escape of beads or loaded reagents from the well, permitting select delivery of certain reagents (e.g., via the use of a semi-permeable membrane), for storage of the well prior to or following further processing, etc.

In some instances, the partition (e.g., well) comprises a plurality of nucleic acid barcode molecules. In some instances, a nucleic acid barcode molecule is provided using a combinatorial approach. In such instances, one or more nucleic acid molecules (which may be comprised in a cell or cell bead) may be partitioned (e.g., in a first set of partitions, e.g., wells or droplets) with one or more first nucleic acid barcode molecules (optionally coupled to a bead). The first nucleic acid barcode molecules or derivative thereof (e.g., complement, reverse complement) may then be attached to the one or more nucleic acid molecules, thereby generating first barcoded nucleic acid molecules, e.g., using the processes described herein. The first nucleic acid barcoded molecules may be partitioned to the first set of partitions such that a nucleic acid barcode molecule, of the first nucleic acid barcode molecules, that is in a partition comprises a barcode sequence that is unique to the partition among the first set of partitions. Each partition may comprise a unique barcode sequence. For example, a set of first nucleic acid barcode molecules partitioned to a first partition in the first set of partitions may each comprise a common barcode sequence that is unique to the first partition among the first set of partitions, and a second set of first nucleic acid barcode molecules partitioned to a second partition in the first set of partitions may each comprise another common barcode sequence that is unique to the second partition among the first set of partitions. Such barcode sequence (unique to the partition) may be useful in determining the cell or partition from which the one or more nucleic acid molecules (or derivatives thereof) originated.

The first barcoded nucleic acid molecules from multiple partitions of the first set of partitions may be pooled and re-partitioned (e.g., in a second set of partitions, e.g., one or more wells or droplets) with one or more second nucleic acid barcode molecules. The second nucleic acid barcode molecules or derivative thereof may then be attached to the first barcoded nucleic acid molecules, thereby generating second barcoded nucleic acid molecules. As with the first nucleic acid barcode molecules during the first round of partitioning, the second nucleic acid barcode molecules may be partitioned to the second set of partitions such that a nucleic acid barcode molecule, of the second nucleic acid barcode molecules, that is in a partition comprises a barcode sequence that is unique to the partition among the second set of partitions. Such barcode sequence may also be useful in determining the cell or partition from which the one or more nucleic acid molecules or first barcoded nucleic acid molecules originated. The second barcoded nucleic acid molecules may thus comprise two barcode sequences (e.g., from the first nucleic acid barcode molecules and the second nucleic acid barcode molecules). Additional barcode sequences may be attached to the second barcoded nucleic acid molecules by repeating the processes any number of times (e.g., in a split-and-pool approach), thereby combinatorically synthesizing unique barcode sequences to barcode the one or more nucleic acid molecules. For example, combinatorial barcoding may comprise at least 1, 2, 3, 4, 5, 6, 7, 8, 9, 10 or more operations of splitting (e.g., partitioning) and/or pooling (e.g., from the partitions). Additional examples of combinatorial barcoding may also be found in International Patent Application Publication No. WO2019/165318 and US Patent Application Publication No. US20200239874, each of which is herein entirely incorporated by reference for all purposes.

Beneficially, the combinatorial barcode approach may be useful for generating greater barcode diversity and synthesizing unique barcode sequences on nucleic acid molecules derived from a cell or partition. For example, combinatorial barcoding comprising three operations, each with 100 partitions, may yield up to 106 unique barcode combinations. In some instances, the combinatorial barcode approach may be helpful in determining whether a partition contained only one cell or more than one cell. For instance, the sequences of the first nucleic acid barcode molecule and the second nucleic acid barcode molecule may be used to determine whether a partition comprised more than one cell. For instance, if two nucleic acid molecules comprise different first barcode sequences but the same second barcode sequences, it may be inferred that the second set of partitions comprised two or more cells.

In some instances, combinatorial barcoding may be achieved in the same compartment. For instance, a unique nucleic acid molecule comprising one or more nucleic acid bases may be attached to a digested polypeptide in successive operations within a partition (e.g., droplet or well). In some instances, all the reagents for barcoding and generating combinatorially barcoded molecules may be provided in a single reaction mixture, or the reagents may be provided sequentially.

In some instances, the well comprises free reagents and/or reagents encapsulated in, or otherwise coupled to or associated with, beads, beads, or droplets.

The wells are provided as a part of a kit. For example, a kit may comprise instructions for use, a microwell array or device, and reagents (e.g., beads). The kit may comprise any useful reagents for performing the processes described herein, e.g., for barcoding of polypeptides and processing.

In some cases, a well comprises a bead, or droplet that comprises a set of reagents that has a similar attribute (e.g., a set of enzymes, a set of minerals, a set of oligonucleotides, a mixture of different barcode molecules, a mixture of identical barcode molecules). In other cases, a bead or droplet comprises a heterogeneous mixture of reagents. In some cases, the heterogeneous mixture of reagents can comprise all components necessary to perform a reaction. In some cases, such mixtures can comprise all components necessary to perform a reaction, except for 1, 2, 3, 4, 5, or more components necessary to perform a reaction. In some cases, such additional components are contained within, or otherwise coupled to, a different droplet or bead, or within a solution within a partition (e.g., microwell) of the system.

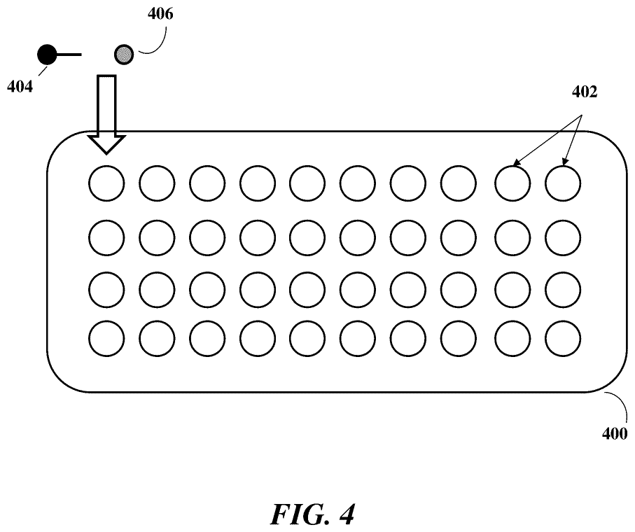

FIG. 4 schematically illustrates an example of a microwell array. The array can be contained within a substrate 400. The substrate 400 comprises a plurality of wells 402. The wells 402 may be of any size or shape, and the spacing between the wells, the number of wells per substrate, as well as the density of the wells on the substrate 400 can be modified, depending on the particular application. In one such example application, a sample molecule 406, which may comprise a cell or cellular components (e.g., peptide, polypeptide) is co-partitioned with a bead 404, which may comprise a peptide and/or nucleic acid barcode molecule coupled thereto. The wells 402 may be loaded using gravity or other loading technique (e.g., centrifugation, liquid handler, acoustic loading, optoelectronic, etc.). In some instances, at least one of the wells 402 contains a single sample molecule 406 (e.g., cell) and a single bead 404.

Reagents may be loaded into a well either sequentially or concurrently. In some cases, reagents are introduced to the device either before or after a particular operation. In some cases, reagents (which may be provided, in certain instances, in droplets, or beads) are introduced sequentially such that different reactions or operations occur at different steps. The reagents (or droplets, or beads) may also be loaded at operations interspersed with a reaction or operation step. For example, beads (or droplets) comprising reagents for fragmenting polynucleotides (e.g., restriction enzymes) and/or other enzymes (e.g., transposases, ligases, polymerases, etc.) may be loaded into the well or plurality of wells, followed by loading of droplets, or beads comprising reagents for attaching nucleic acid barcode molecules to a digested polypeptide. Reagents may be provided concurrently or sequentially with a sample, e.g., a cell or cellular components. Accordingly, use of wells may be useful in performing multi-step operations or reactions.

Provided is a method comprising providing a plurality of digested polypeptides from a single cell in a partition (e.g., a well), wherein the partition comprises a plurality of nucleic acid barcode molecules unique to the partition, and providing a reducing agent and a conjugation reagent to the partition to generate a plurality of polypeptide-oligonucleotide conjugates by coupling the plurality of digested polypeptides to the plurality of nucleic acid barcode molecules.

In some embodiments, the nucleic acid barcode molecules and other reagents are associated with a bead or in a droplet. In some embodiments, the beads, or droplets are loaded into a partition (e.g., a microwell) before, after, or concurrently with the loading of a cell, such that each cell is contacted with a different bead, or droplet. This technique may be used to attach a unique nucleic acid barcode molecule to digested polypeptides obtained from each cell. In some instances, the partition (e.g., microwell) may comprise a bead which has coupled thereto a plurality of nucleic acid barcode molecules. In some embodiments, the resulting barcoded polypeptide conjugate is removed from the partition, and in some instances, pooled and processed. In such cases, the nucleic acid barcode sequences are used to trace the origin of the digested polypeptide. For example, polypeptides conjugated to nucleic acid barcode molecules with identical barcodes may be determined to originate from the same cell or partition, while polypeptides conjugated to nucleic acid barcode molecules with different barcodes may be determined to originate from different cells or partitions.

In some embodiments, the samples or reagents are loaded in the wells or microwells using a variety of approaches. In some embodiments, the samples (e.g., a cell, cell bead, or cellular component) or reagents (as described herein) are loaded into the well or microwell using an external force, e.g., gravitational force, electrical force, magnetic force, or using mechanisms to drive the sample or reagents into the well, e.g., via pressure-driven flow, centrifugation, optoelectronics, acoustic loading, electrokinetic pumping, vacuum, capillary flow, etc. In certain cases, a fluid handling system may be used to load the samples or reagents into the well. The loading of the samples or reagents may follow a Poissonian distribution or a non-Poissonian distribution, e.g., super Poisson or sub-Poisson. The geometry, spacing between wells, density, and size of the microwells may be modified to accommodate a useful sample or reagent distribution; for instance, the size and spacing of the microwells may be adjusted such that the sample or reagents may be distributed in a super-Poissonian fashion.

In some embodiments, the droplet or bead is partitioned into a well. The droplets may be selected or subjected to pre-processing prior to loading into a well. For instance, the droplets may comprise cells, and only certain droplets, such as those containing a single cell (or at least one cell), may be selected for use in loading of the wells. Such a pre-selection process may be useful in efficient loading of single cells, such as to obtain a non-Poissonian distribution, or to pre-filter cells for a selected characteristic prior to further partitioning in the wells. Additionally, the technique may be useful in obtaining or preventing cell doublet or multiplet formation prior to or during loading of the microwell.

In some instances, the wells comprise nucleic acid barcode molecules attached thereto. In some instances, the nucleic acid barcode molecules are attached to a surface of the well (e.g., a wall of the well). In some instances, the nucleic acid barcode molecules are attached to a droplet or bead that has been partitioned into the well. In some instances, the nucleic acid barcode molecule (e.g., a partition barcode sequence) of one well differs from the nucleic acid barcode molecule of another well, which can permit identification of the contents contained with a single partition or well.

In some instances, the nucleic acid barcode molecule comprises a spatial barcode sequence that can identify a spatial coordinate of a well, such as within the well array or well plate. In some instances, the nucleic acid barcode molecule comprises a unique molecular identifier for individual molecule identification. In some instances, the nucleic acid barcode molecule is configured to couple to a digested polypeptide from a sample or cell distributed in the well. In some instances, the nucleic acid barcode molecules are releasable from the microwell. In some instances, nucleic acid barcode molecules are releasable from the bead or droplet. In some instances, the nucleic acid barcode molecules comprise a chemical cross-linker which may be cleaved upon application of a stimulus (e.g., photo-, magnetic, chemical, biological, stimulus).

In some instances, the well is loaded with a sample and reagents, simultaneously or sequentially. When cells or cell beads are loaded, the well may be subjected to washing, e.g., to remove excess cells from the well, microwell array, or plate. Similarly, washing may be performed to remove excess beads or other reagents from the well, microwell array, or plate. In the instances where live cells are used, the cells may be lysed in the individual partitions to release the intracellular components or cellular analytes. Alternatively, the cells may be fixed or permeabilized in the individual partitions. In some instances, a digested polypeptide is coupled to a support, e.g., on a surface of the microwell, on a solid support (e.g., bead), or they may be collected for further downstream processing. For instance, after cell lysis, a digested polypeptide is provided in individual droplets or other partitions for barcoding. Alternatively, or in addition to, the digested polypeptides from a single cell are barcoded in the well (e.g., using a plurality of nucleic acid barcode molecules that are releasable or on a surface of the microwell). In some instances, further processing occurs in the well, or the polypeptide-oligonucleotide conjugates are collected from the individual partitions and subjected to further processing outside the partition.

### C. Cell Beads

In some embodiments, in addition to or as an alternative to droplet-based partitioning, biological particles (e.g., cells) are comprised within (e.g., encapsulated within) a particulate material to form a “cell bead”. Methods and compositions drawn to cell beads and the like are described further in U.S. Patent Application No. 2018/0216162 and 2018/0216162, which are hereby incorporated by reference in their entirety. In some instances, cell beads comprising nucleic acid molecules may be barcoded. Methods and systems for barcoding cell beads are further described in U.S. Patent Application Publication No. 2019/0330694, which is hereby incorporated by reference in its entirety.

Provided herein is a method comprising providing a plurality of cell beads and a plurality of barcode beads, wherein (i) a cell bead of the plurality of cell beads comprises a plurality of polypeptides from a single cell, and (ii) a barcode bead of the plurality of barcode beads comprises a plurality of nucleic acid barcode molecules for barcoding the plurality of polypeptides, wherein the cell bead is provided with an enzyme to generate a digested polypeptide; contacting the digested polypeptide with a conjugation reagent; and partitioning the plurality of cell beads and the plurality of barcode beads into a plurality of partitions, wherein upon partitioning, a partition of the plurality of partitions comprises the cell bead, the barcode bead and a reducing agent to release a nucleic acid barcode molecule of the plurality of nucleic acid barcode molecule coupled to the barcode bead; thereby generating a polypeptide-oligonucleotide conjugate comprising the digested polypeptide and the nucleic acid barcode molecule. In some instances, the plurality of cell beads are partitioned and/processed as described in Section II.B.

In some instances, the cell bead comprises the single cell, and wherein the cell bead comprising the single cell is subjected to conditions sufficient to lyse the cell to provide the plurality of polypeptides. In some instances, the cell bead comprising the single cell or contents thereof is exposed to a lysis agent. In some embodiments, the cell bead is exposed to trypsin. In some instances, the cell bead is not dissolvable. In some embodiments, the cell bead is exposed to trypsin. In some instances, the cell bead is not dissolvable by the reducing agent. In some embodiments, a cell bead may be dissolvable, disruptable, or degradable. In some cases, a cell bead may not be degradable.

In some instances, the cell bead is generated by providing a plurality of cells and a plurality of polymeric or gel precursors; partitioning the plurality of cells and the plurality of polymeric or gel precursors into a plurality of partitions, wherein upon partitioning, a partition of the plurality of partitions comprises a cell of the plurality of cells and at least a portion of the polymeric or gel precursors; and subjecting the partitions to conditions suitable for cross-linking or polymerizing the polymeric or gel precursors to generate the cell bead, wherein the cell bead encapsulates the cell. In some instances, the cell bead includes or is enclosed within a gel or polymer matrix within the partition.

In some instances, a cell bead comprises a biological particle (e.g., a cell) or macromolecular constituents (e.g., RNA, DNA, proteins, polypeptides, peptides etc.) of a biological particle. In some instances, a cell bead comprises a single cell or multiple cells, or a derivative of the single cell or multiple cells. For example, after lysing and washing the cells, inhibitory components from cell lysates can be washed away and the macromolecular constituents can be bound as cell beads. Systems and methods disclosed herein can be applicable to both cell beads (and/or droplets or other partitions) containing biological particles and cell beads (and/or droplets or other partitions) containing macromolecular constituents of biological particles. Cell beads may be or include a cell, cell derivative, cellular material and/or material derived from the cell in, within, or encased in a matrix, such as a polymeric matrix. In some cases, a cell bead comprises a live cell. In some instances, the live cell may be capable of being cultured when enclosed in a gel or polymer matrix, or of being cultured when comprising a gel or polymer matrix. In some instances, the polymer or gel may be diffusively permeable to certain components and diffusively impermeable to other components (e.g., macromolecular constituents).

In some instances, a cell bead provides certain potential advantages of being more storable and more portable than droplet-based partitioned biological particles. In some instances, a cell bead is partitioned into a droplet to introduce reagents to the contents of the cell bead. In some embodiments, to generate polypeptide-oligonucleotide conjugates, a conjugation reagent (e.g., a diazo transfer reagent) is added to the digested protein and then quenched. In some cases, a constrained-cyclooctyne cannot be activated by copper, and thus by performing the diazo transfer first, excess diazo conjugation reagent is quenched in the absence of copper. In some instances, this quenching allows conjugation of the azido-peptide specifically to the alkyne-oligo without reacting the excess constrained-alkyne. In some aspects, use of cell beads which allows reagents to be flowed to the cell bead and its contents is an alternative to the step-wise workflow (e.g., providing conjugation reagent, quenching, providing copper). In some aspects, use of cell beads which allows removal of excess reagents by washing.

In some cases, air knife droplet or aerosol generators may be used to dispense droplets of precursor fluids into gelling solutions in order to form cell beads that include individual biological particles or small groups of biological particles. Likewise, membrane-based encapsulation systems may be used to generate cell beads comprising encapsulated biological particles as described herein. Microfluidic systems of the present disclosure, such as that shown in FIG. 1B, may be readily used in encapsulating biological particles (e.g., cells) as described herein. Examples of methods for encapsulating biological particles (e.g., cells) are also further described in U.S. Patent Application Pub. No. 2015/0376609 and 2018/0216162, which are hereby incorporated by reference in their entirety. In particular, and with reference to FIG. 1B, the aqueous fluid 112 comprising (i) the biological particles 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the bead that includes the entrained biological particles. Examples of polymer precursor/initiator pairs include those described in U.S. Patent Application Publication No. 2014/0378345, which is entirely incorporated herein by reference for all purposes.

In some cases, encapsulated biological particles are selectively releasable from the cell bead, such as through passage of time or upon application of a particular stimulus, that degrades the bead sufficiently to allow the biological particles (e.g., cell), or its other contents to be released from the bead, such as into a partition (e.g., droplet). Examples of stimuli suitable for degradation of the bead are described in U.S. Patent Application Publication No. 2014/0378345, which is entirely incorporated herein by reference for all purposes.

In some instances, the polymer or gel is diffusively permeable to chemical or biochemical reagents. In some instances, the polymer or gel is diffusively impermeable to macromolecular constituents of the biological particle. In this manner, the polymer or gel may act to allow the biological particle to be subjected to chemical or biochemical operations while spatially confining the macromolecular constituents to a region of the droplet defined by the polymer or gel. In some instances, the polymer or gel is polymerized or gelled via a passive mechanism. In some instances, the polymer or gel is stable in alkaline conditions or at elevated temperature. In some instances, the polymer or gel has mechanical properties similar to the mechanical properties of the bead. For instance, the polymer or gel may be of a similar size to the bead. In some instances, the polymer or gel may have a mechanical strength (e.g., tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. In some instances, the pore size is chosen to maintain diffusive permeability to exogenous chemicals such as sodium hydroxide (NaOH) and/or endogenous chemicals such as inhibitors. In some instances, the polymer or gel is biocompatible. In some instances, the encapsulation of biological particles constitutes the partitioning of the biological particles into which other reagents are co-partitioned. Alternatively, or in addition, encapsulated biological particles may be readily deposited into other partitions (e.g., droplets) as described above.

### D. Sample Processing

In some embodiments, the biological particles (e.g., single cells) is lysed in the partitions (e.g., in the droplets or wells). In some embodiments, proteins are denatured in the partition (e.g., in the droplet or well). In some instances, a partition comprises a lysis agent. In some instances, a droplet comprises a lysis agent. Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents may additionally or alternatively be co-partitioned with the biological particles to cause the release of the biological particle's contents into the partitions. For example, in some cases, surfactant-based lysis solutions may be used to lyse cells. In some cases, lysis solutions may include non-ionic surfactants such as, for example, TritonX-100 and Tween 20. In some cases, lysis solutions comprise ionic surfactants such as, for example, sarcosyl and sodium dodecyl sulfate (SDS). Electroporation, thermal, acoustic or mechanical cellular disruption may also be used in certain cases, e.g., non-emulsion-based partitioning such as encapsulation of biological particles that may be in addition to or in place of droplet partitioning, where any pore size of the encapsulate is sufficiently small to retain nucleic acid fragments of a given size, following cellular disruption.

Alternatively, or in addition to, the lysis agents co-partitioned with the biological particles described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles (e.g., a cell or a nucleus in a polymer matrix), the biological particles may be exposed to an appropriate stimulus to release the biological particles or their contents from a co-partitioned bead. For example, in some cases, a chemical stimulus may be co-partitioned along with an encapsulated biological particle to allow for the degradation of the bead and release of the cell or its contents into the larger partition. In some cases, this stimulus may be the same as the stimulus described elsewhere herein for release of nucleic acid barcode molecules from their respective bead. In alternative examples, this may be a different and non-overlapping stimulus, in order to allow an encapsulated biological particle to be released into a partition at a different time from the release of nucleic acid barcode molecules into the same partition. For a description of methods, compositions, and systems for encapsulating cells (also referred to as a “cell bead”), see, e.g., U.S. Pat. No. 10,428,326 and U.S. Pat. Pub. 20190100632, which are each incorporated by reference in their entirety.

In some instances, cells are lysed in the individual partitions to release the intracellular components or cellular analytes. Alternatively, the cells are fixed or permeabilized in the individual partitions.

In some embodiments, cells are lysed and proteins digested into short peptides in the partitions. In some embodiments, cells are lysed and proteins are digested prior to coupling a digested polypeptide to the nucleic acid barcode molecule.

In some instances, the contents of the single cells are treated with an enzyme to digest the proteins into fragments. In some instances, the enzyme is a digestion enzyme. In some instances, the enzyme is a protease. In some instances, the enzyme is trypsin. In some instances, a peptide, polypeptide, or protein is fragmented by proteinase K, or optionally, a thermolabile version of proteinase K to enable rapid inactivation. In some instances, the contents of the single cells are treated with proteinase K, trypsin, chymotrypsin, pepsin, thermolysin, thrombin, furin, endopeptidase, papain, pepsin, subtilisin, elastase, enterokinase, Endoproteinase LysC, Endoproteinase AspN, Endoproteinase GluC. In some instances, the contents of the single cells are treated with chemical agents to digest the proteins into fragments. In some instances, a chemical reagent cleaves at a specific amino acid residue. For example, cyanogen bromide hydrolyzes peptide bonds at the C-terminus of methionine residues. In some instances, a chemical reagent fragments polypeptides or proteins into smaller peptides in the partition. In some instances, the chemical reagent is cyanogen bromide (CNBr), hydroxylamine, hydrazine, formic acid, BNPS-skatole [2-(2-nitrophenylsulfenyl)-3-methylindole], or iodosobenzoic acid.

In some instances, following enzymatic or chemical treatment, the resulting digested polypeptides comprise fragments that are from about 10 amino acids to about 70 amino acids, from about 10 amino acids to about 60 amino acids, from about 10 amino acids to about 50 amino acids, about 10 to about 40 amino acids, from about 10 to about 30 amino acids, from about 20 amino acids to about 70 amino acids, from about 20 amino acids to about 60 amino acids, from about 20 amino acids to about 50 amino acids, about 20 to about 40 amino acids, from about 20 to about 30 amino acids, from about 30 amino acids to about 70 amino acids, from about 30 amino acids to about 60 amino acids, from about 30 amino acids to about 50 amino acids, or from about 30 amino acids to about 40 amino acids.

### E. Conjugation

In some embodiments, digested polypeptides from a biological particle (e.g., cell) and nucleic acid barcode molecules coupled to a particle in a partition are contacted with a conjugation reagent and a reducing reagent to generate a plurality of polypeptide-oligonucleotide conjugates. In some embodiments, the reducing agent and/or the conjugation reagent is provided to the droplet population using an additional emulsion comprising reducing agent and/or the conjugation reagent. In some embodiments, the additional emulsion comprises additional droplets comprising the reducing agent and/or the conjugation reagent. In some embodiments, the conjugation reagent is transferred to the droplet population prior to the releasing the nucleic acid barcode molecules from the particle. In some instances, the conjugation reagent is transferred to the droplet population with the reducing agent. In some instances, the reducing agent is transferred to the droplet population via micelles after the conjugation reagent is transferred to the droplet population.

In some embodiments, the reducing agent is provided after the conjugation reagent is provided to the sample. In some embodiments, the nucleic acid barcode molecules are released (e.g., made available for coupling to the digested polypeptides) after the conjugation reagent is provided to the sample. In some embodiments, the conjugation reagent is provided to the sample after treatment with trypsin. In some embodiments, reaction of the digested polypeptides with the conjugation reagent generates excess reagents. In some embodiments, excess reagents are washed and removed. In some embodiments, the conjugation reagent, the reducing agent, and the activating agent (e.g., copper) are provided in a step-wise manner for generating polypeptide-oligonucleotide conjugates.

In some embodiments, nucleic acid barcode molecules are releasable from the beads upon the application of a particular stimulus to the beads. In some embodiments, nucleic acid barcode molecules are releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In some cases, the stimulus is a photo-stimulus, e.g., through cleavage of a photo-labile linkage that releases the nucleic acid barcode molecules. In other cases, a thermal stimulus is used, where elevation of the temperature of the beads environment will result in cleavage of a linkage or other release of the nucleic acid barcode molecules from the beads. In some instances, a chemical stimulus that cleaves a linkage of the nucleic acid barcode molecules to the particles (e.g., beads) is used, or otherwise results in release of the nucleic acid barcode molecules from the beads. In one case, such compositions include the polyacrylamide matrices described above for encapsulation of biological particles and may be degraded for release of the attached nucleic acid barcode molecules through exposure to a reducing agent, such as DTT. In some instances, the reducing agent is a disulfide cleaving reagent. In some instances, the reducing agent is dithiothreitol (DTT), or a functional derivative thereof.

In some instances, the droplets are subjected to conditions sufficient to transfer a conjugation reagent to the droplet population. In some instances, the droplets are subjected to conditions sufficient to transfer a reducing agent to the droplet population. In some instances, a reducing agent releases the nucleic acid barcode molecule coupled to the particle. In some instances, the reducing agent dissolves the particle and/or releases at least a portion of the plurality of nucleic acid barcode molecules from the particle. In some embodiments, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. See, e.g., PCT/US2014/044398, which is hereby incorporated by reference in its entirety.

In some instances, the droplets are subjected to conditions sufficient to transfer a conjugation reagent to the droplet population via micelles. In some instances, the reducing agent and/or the conjugation reagent is in a water-in-oil emulsion and the micelles are water-in-oil micelles. In some instances, the reducing agent and/or the conjugation reagent is provided in a liposome. In some embodiments, the transfer of the reagent to a particle (e.g., the reducing agent and/or the conjugation reagent) is mediated by a “cage” of molecules around the reagent. In some instances, a surfactant comprising a surfactant head and a surfactant tail is used. In some instances, a molecule with a head and a tail which comprise different solubility is used to transfer reagents to a particle comprising the digested polypeptides. In some instances, the surfactant head is more soluble in a fluid in the interior of the micelle. In an example, the solubility of the head may be greater in water and a tail may be more soluble in the oil. In some instances, a micelle may comprise an aggregate of surfactant molecules dispersed in a fluid. The micelle or liposome may be spherical, may comprise ellipsoids, may be cylindrical, may be bilayers, etc. The size and shape of the micelle may be changed by adjusting surfactant and/or fluid conditions such as pH, temperature, surfactant concentration in the fluid, solute concentration in the fluid, reagent concentration in the fluid, ionic strength of the fluid, etc. Systems and methods for reagent transfer and droplet and/or emulsion generation by agitation are described in U.S. Patent Application Publication No. 2022/0025438 and 2014/0155295, which are entirely incorporated herein by reference for all purposes.

In some embodiments, the transfer of the reagent to a particle (e.g., the reducing agent and/or the conjugation reagent) is a transfer from a first droplet to a second droplet within a mixture via diffusion, such as diffusion of the reagents from the first droplet to the second droplet. Such diffusion may be facilitated by a micelle. In some cases, alternatively or in addition to diffusion, other forms of mass transfer or transport phenomena such as convection, heat-mediated mass transfer, turbulent mass transfer (e.g., caused by mixing, agitation, vortexing, shear stress, and/or fluid flow), and/or other forms of mass transfer may also be involved. In some cases, a method of processing a droplet may additionally comprise, generating a mixture comprising the first droplet and the second droplet, after providing a first droplet and a second droplet. The reagent may be transferred from the first droplet to the second droplet within the mixture. The reagent may be transferred from the first droplet to the second droplet by any mode described herein.

In some instances, the micelles are water-in-fluorinated oil micelles. In some cases, the micelles are formed using a surfactant. In some instances, the reducing agent is transferred to the droplet population via micelles. In some instances, the reducing agent is transferred to the droplet population with copper via micelles. In some instances, a micelle can be any suitable surfactant-based transporter. In some instances, the micelle is a water-in-oil micelle.

In some embodiments, the method comprises subjecting a droplet population to conditions sufficient to transfer the reagent from an additional droplet population comprising the reagent. In some cases, the reagent is a reducing agent. In some examples, the reagent comprises dithiothreitol (DTT). The reagent may be transported between the first droplet population and the second droplet population using the methods described elsewhere herein, such as with the aid of a micelle. In some embodiments, the transfer of DTT to the droplet dissolves the beads. The transfer of a reducing agent (e.g., DTT) to the droplet may dissolve the beads and/or facilitate release of nucleic acid barcode molecules coupled thereto in the droplets for coupling to digested polypeptides in the partition.

In some embodiment, the micelles comprising the reagent to be transferred to a particle (e.g., the reducing agent and/or the conjugation reagent) are generated by applying energy. In some embodiment, the micelles comprising the reagent to be transferred to a particle (e.g., the reducing agent and/or the conjugation reagent) are generated by applying a stimulus, such as agitation, such as vertexing to compartmentalize the beads into the droplets. In some embodiments, a first fluid that is an aqueous solution comprising reagent(s) is in a container and a second fluid comprising oil is added to the container. In some embodiments, a surfactant is added to the container and the container is agitated (e.g., vortexed) to generate the micelles comprising the reagent to be transferred to a particle (e.g., the reducing agent and/or the conjugation reagent).

In some embodiments, the nucleic acid barcode molecule (e.g., released from the particle) is covalently coupled to the digested polypeptide. In some embodiments, the nucleic acid barcode molecule is coupled to a reactive group present on the digested polypeptide. In some instances, the nucleic acid barcode molecule is attached to a lysine of the digested polypeptide to form a polypeptide-oligonucleotide conjugate. In some instances, the nucleic acid barcode molecule is coupled to an internal amino acid of the digested polypeptide. In some instances, the nucleic acid barcode molecule is attached to a cysteine of the digested polypeptide to form a polypeptide-oligonucleotide conjugate. In some examples, attachment to the cysteine is performed using maleimide chemistry.

In some embodiments, the nucleic acid barcode molecule (e.g., released from the particle) is coupled to the digested polypeptide using a chemical bioconjugation reaction. In some embodiments, the conjugation reagent comprises a heterobifunctional agent. In some instances, the conjugation reagent comprises click-reactive reactive handles. In some instances, the conjugation reagent enables subsequent click chemistry. In some embodiments, the nucleic acid barcode molecule (e.g., released from the particle) is coupled to the digested polypeptide using a copper catalyzed reaction of an azide and alkyne to form a triazole (copper-catalyzed alkyne-azide cycloaddition (CuAAc) or Huisgen 1, 3-dipolar cycloaddition), strain-promoted alkyne-azide cycloaddition (SPAAC), reaction of a diene and dienophile (Diels-Alder), strain-promoted alkyne-nitrone cycloaddition, reaction of a strained alkene with an azide, tetrazine or tetrazole, alkene and azide [3+2] cycloaddition, alkene and tetrazine inverse electron demand Diels-Alder (IEDDA) reaction (e.g., m-tetrazine (mTet) or phenyl tetrazine (pTet) and trans-cyclooctene (TCO); or pTet and an alkene), alkene and tetrazole photoreaction, or Staudinger ligation of azides and phosphines. In some embodiments, the nucleic acid barcode molecule (e.g., released from the particle) is coupled to the digested polypeptide using m-tetrazine or phenyl tetrazine (pTet). In some embodiments, the nucleic acid barcode molecule (e.g., released from the particle) is coupled to the digested polypeptide using iEDDA click chemistry reaction. In some embodiments, the nucleic acid barcode molecule (e.g., released from the particle) is coupled to the digested polypeptide using a bifunctional click chemistry reagent. In some embodiments, the nucleic acid barcode molecule (e.g., released from the particle) is coupled to the digested polypeptide using an alkyne-NHS ester (acetylene-PEG-NHS ester) reagent or alkyne-benzophenone to generate an alkyne-labeled polypeptide. In some embodiments, the alkyne is a strained alkyne. In some embodiments, the nucleic acid barcode molecule (e.g., released from the particle) is coupled to the digested polypeptide using cyclooctynes (e.g., Dibenzocyclooctyl (DBCO)).

In some instances, the coupling of the nucleic acid barcode molecule to the digested polypeptide is catalyzed by an activating agent. In some instances, the coupling of the nucleic acid barcode molecule to the digested polypeptide is copper catalyzed. In some instances, digested polypeptides are derivatized with reactive groups (e.g., click chemistry moieties). In some instances, digested polypeptides are coupled to a nucleic acid barcode molecule (e.g., released from the particle in the partition) using the complementary click chemistry moiety. In some instances, a digested polypeptide is derivatized with alkyne and mTet moieties and coupled to a nucleic acid barcode molecule using azide and TCO.

In some instances, prior to coupling the nucleic acid barcode molecule to the digested polypeptide, the digested polypeptide is modified to comprise an azide group. In certain embodiments, the modification comprises contacting the digested polypeptide with a diazo transfer reagent in the presence of a copper salt and a base. As used herein, “diazo transfer reagent” refers to a reagent that comprises a N₃ moiety and that aids in the conversion of a primary amine into an azide functional group. In certain embodiments, the copper salt is CuSO₄. In certain embodiments, the base is K₂CO₃. In certain embodiments, the diazo transfer reagent is imidazole-1-sulfonyl azide hydrochloride.

In certain embodiments, after contacting the digested polypeptide with a diazo transfer reagent in the presence of a copper salt and a base to form an azide-modified polypeptide, unreacted diazo transfer reagent remains. To quench such unreacted diazo transfer reagent, the unreacted diazo transfer reagent is contacted with a cyclooctyne. In certain embodiments, the cyclooctyne is a dibenzocyclooctyne. In certain embodiments, the dibenzocyclooctyne has the following structure:

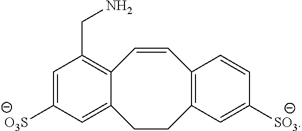

The remaining unreacted diazo transfer reagent undergoes strain-promoted alkyne-azide cycloaddition (SPAAC) with the cyclooctene, thereby quenching the unreacted diazo transfer reagent. In certain embodiments, the strain-promoted alkyne-azide cycloaddition (SPAAC) results in the formation of a triazolyl.

In some instances, prior to coupling the nucleic acid barcode molecule to the digested polypeptide, the nucleic acid barcode molecule is modified to comprise an alkyne. In some embodiments, the alkyne is introduced during or immediately after oligonucleotide synthesis. Modified oligonucleotides can be prepared in accordance with the methods disclosed in Obeid et al., JACS, vol. 135, 42, 2013, which is incorporated herein in its entirety. In certain embodiments, the nucleic acid barcode molecule is modified to contain an alkyne through modification with 5-octadinyl dU.

In some instances, the alkyne functionality is incorporated into the nucleic acid barcode molecule prior to its conjugation to a solid support (e.g., a bead). In some instances, the alkyne group is introduced by incorporating an alkyne phosphoramidite. In some instances, a terminal modification or an internal alkyne modifier is incorporated directly into the nucleic acid barcode molecule using phosphoramidite coupling chemistry. In some instances, the alkyne functionality may be introduced post-synthetically by reacting a primary amine-modified oligonucleotide with an alkyne-containing N-hydroxysuccinimide (NHS) ester reagent. In some instances, an orthogonal functional group is used to enable site-specific conjugation. Examples of suitable functional groups include, but are not limited to, a primary amine, biotin, or an azide group. Examples of reagents for coupling moieties for click chemistry coupling include alkyne-NHS reagents for lysine labeling and alkyne-benzophenone reagents for photoaffinity labeling. In some embodiments, examples of suitable functional groups are described in U.S. Patent Application Publication No. 2020/0348307, which is hereby incorporated by reference in its entirety.

In certain embodiments, the azide moiety of the digested polypeptide is conjugated to the alkyne moiety of the nucleic acid barcode molecule by means of copper-catalyzed alkyne-azide cycloaddition (CuAAC). In certain embodiments, CuAAC results in the formation of a triazolyl.

In certain embodiments, the copper-catalyzed CuAAC reaction is catalyzed through application of copper sulfate. In certain embodiments, energy, such as heat or light, is used to reduce the Cu (II) to Cu (I). In some aspects, the method is performed in the presence of a photoinitiator molecule. A photoinitiator (e.g., Irgacure 2959) creates reactive species (e.g., free radicals, cations, or anions) when exposed to UV light. The photoinitiator generates radicals, which reduce Cu (II) to Cu (I). The Cu (I) catalyzes the 1,3-dipolar cycloaddition reaction. The generated Cu (I) catalyzes the Click reaction between the azide group of the digested polypeptide with the alkyne group of the nucleic acid barcode molecule to form a Click product comprising a 1,2,3-triazole group. An example of copper-catalyzed alkyne-azide click chemistry reaction is shown below:

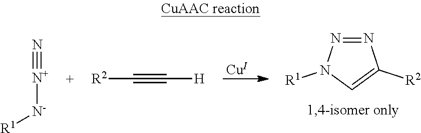

In some instances, a plurality of polypeptide-oligonucleotide conjugates are released from the emulsion prior to immobilizing the plurality of polypeptide-oligonucleotide conjugates to the surface. In some embodiments, the generated polypeptide-oligonucleotide conjugate is joined to a surface. In some instances, the surface is a support. In some embodiments, a plurality of polypeptide-oligonucleotide conjugates are joined to a support. In some embodiments, the support is a bead. In some examples, supports can include, but are not limited to, a ceramic bead, a polystyrene bead, a polymer bead, a polyacrylate bead, a methylstyrene bead, an agarose bead, a cellulose bead, a dextran bead, an acrylamide bead, a porous bead, a paramagnetic bead, a glass bead, a controlled pore bead, a silica-based bead, or any combinations thereof. In some instances, the bead is a polystyrene bead, a polymer bead, an agarose bead, an acrylamide bead, a solid core bead, a porous bead, a paramagnetic bead, glass bead, or a controlled pore bead. In some embodiments, a polypeptide-oligonucleotide conjugate is immobilized to the surface (e.g., of a bead) via a thiol group. In some instances, a plurality of polypeptide-oligonucleotide conjugates are immobilized to the surface by Michael-type addition of a thiol to a maleimide group. In some instances, the thiol is a thiol in a cysteine group in the digested polypeptide of the polypeptide-oligonucleotide conjugate.

In some embodiments, the generated polypeptide-oligonucleotide conjugate is joined to a surface of a solid support (e.g., substrate surface). In some examples, the support is any suitably porous or non-porous support surface including, but not limited to, a bead, a microbead, an array, a glass surface, a silicon surface, a plastic surface, a filter, a membrane, nylon, a silicon wafer chip, a flow cell, a flow through chip, a biochip including signal transducing electronics, a microtiter well, an ELISA plate, a spinning interferometry disc, a nitrocellulose membrane, a nitrocellulose-based polymer surface, a nanoparticle, or a microsphere. In some embodiments, the support comprises a material including, but not limited to, acrylamide, agarose, cellulose, nitrocellulose, glass, gold, quartz, polystyrene, polyethylene vinyl acetate, polypropylene, polymethacrylate, polyethylene, polyethylene oxide, polysilicates, polycarbonates, Teflon, fluorocarbons, nylon, silicon rubber, polyanhydrides, polyglycolic acid, polyactic acid, polyorthoesters, functionalized silane, polypropylfumerate, collagen, glycosaminoglycans, polyamino acids, or any combination thereof. In some embodiments, the generated polypeptide-oligonucleotide conjugate is joined to a film, membrane, bottles, dishes, fibers, woven fibers, shaped polymers such as tubes, particles, beads, microparticles, or any combination thereof.

In some embodiments, the surface is a flow cell. Flow cell configurations may vary among different next generation sequencing platforms. In some instances, the flow cell is a planar optically transparent surface. In some embodiments, the surface is a microscope slide. In some instances, the slide comprises a lawn of oligonucleotide anchors bound to its surface. In some embodiments, the surface is a bead, which may refer to an individual bead or a plurality of beads. In some embodiments, the bead is compatible with a next generation sequencing platform configured for downstream analysis. In some embodiments, a surface is an agarose bead, a paramagnetic bead, a polystyrene bead, a polymer bead, an acrylamide bead, a solid core bead, a porous bead, a glass bead, or a controlled pore bead. In some embodiments, a bead is coated with a binding functionality (e.g., amine group, affinity ligand such as streptavidin for binding to biotin labeled polypeptide, antibody) to facilitate binding to a polypeptide.

In some embodiments, the generated polypeptide-oligonucleotide conjugate is joined to the solid support, directly or indirectly, including covalent and non-covalent interactions, or any combination thereof. In some embodiments, the generated polypeptide-oligonucleotide conjugate is joined to the solid support by a ligation reaction. In some embodiments, the generated polypeptide-oligonucleotide conjugate is joined to the solid support via an agent or coating to facilitate joining, either direct or indirectly, the peptide to the solid support. In some instances, the agent is an affinity molecule. In some instances, the agent is an azide group, which group can react with an alkynyl group in another molecule to facilitate association or binding between the solid support and the other molecule.

In some embodiments, the generated polypeptide-oligonucleotide conjugate is joined to the solid support using methods referred to as “click chemistry.” For this purpose, any reaction which is rapid and substantially irreversible can be used to attach proteins, polypeptides, or peptides to the solid support. In some embodiments, the generated polypeptide-oligonucleotide conjugate is joined to the solid support using reactions that include the copper catalyzed reaction of an azide and alkyne to form a triazole (copper-catalyzed alkyne-azide cycloaddition (CuAAc) or Huisgen 1, 3-dipolar cycloaddition), strain-promoted alkyne-azide cycloaddition (SPAAC), reaction of a diene and dienophile (Diels-Alder), strain-promoted alkyne-nitrone cycloaddition, reaction of a strained alkene with an azide, tetrazine or tetrazole, alkene and azide [3+2] cycloaddition, alkene and tetrazine inverse electron demand Diels-Alder (IEDDA) reaction (e.g., m-tetrazine (mTet) or phenyl tetrazine (pTet) and trans-cyclooctene (TCO); or pTet and an alkene), alkene and tetrazole photoreaction, Staudinger ligation of azides and phosphines, and various displacement reactions, such as displacement of a leaving group by nucleophilic attack on an electrophilic atom (Horisawa, Front Physiol (2014). 5:457; Knall, Hollauf et al., Tetrahedron Lett (2014) 55 (34): 4763-4766). In some cases, examples of displacement reactions include reaction of an amine with: an activated ester, an N-hydroxysuccinimide ester, an isocyanate, an isothioscyanate, an aldehyde, an epoxide, or the like.

In some embodiments, the generated polypeptide-oligonucleotide conjugate is joined to the solid support using a functional group capable of formation by reaction of two complementary reactive groups, for example a functional group which is the product of one of the foregoing “click” reactions. In various embodiments, a functional group can be formed by reaction of an aldehyde, oxime, hydrazone, hydrazide, alkyne, amine, azide, acylazide, acylhalide, nitrile, nitrone, sulfhydryl, disulfide, sulfonyl halide, isothiocyanate, imidoester, activated ester (e.g., N-hydroxysuccinimide ester, pentynoic acid STP ester), ketone, α,β-unsaturated carbonyl, alkene, maleimide, a-haloimide, epoxide, aziridine, tetrazine, tetrazole, phosphine, biotin or thiirane functional group with a complementary reactive group. An exemplary reaction is a reaction of an amine (e.g., primary amine) with an N-hydroxysuccinimide ester or isothiocyanate.

In some embodiments, the functional group comprises an alkene, ester, amide, thioester, disulfide, carbocyclic, heterocyclic or heteroaryl group. In further embodiments, the functional group comprises an alkene, ester, amide, thioester, thiourea, disulfide, carbocyclic, heterocyclic or heteroaryl group. In other embodiments, the functional group comprises an amide or thiourea. In some more specific embodiments, functional group is a triazolyl functional group, an amide, or thiourea functional group. In some embodiments, the generated polypeptide-oligonucleotide conjugate is joined to the solid support using a thiol-maleimide reaction.

In some embodiments, the polypeptide-oligonucleotide conjugate is immobilized to the surface (solid support) by a Michael-type addition of a thiol to a maleimide group. For example, in certain embodiments, the polypeptide-oligonucleotide conjugate can comprise a thiol group, such as a cysteine amino acid. In certain other embodiments, the polypeptide-oligonucleotide conjugate can be chemically modified to comprise a thiol group, such as through introduction of a thiol-containing phosphoramidite. In certain embodiments, the thiol of the oligonucleotide proceeds in a Michael-type addition with a maleimide-functionalized bead to form a sulfide bond.

In certain embodiments, a heterobifunctional crosslinker is used to covalently attach a thiol in the polypeptide-oligonucleotide conjugate to a surface. In certain embodiments, the heterobifunctional crosslinker is N-hydroxysuccinimide ester or succinimidyl 4-[maleimidophenyl] butyrate (SMPB). In certain embodiments, the heterobifunctional crosslinker links the thiol-containing polypeptide-oligonucleotide conjugate to an amine-derivatized surface.

In some embodiments, the generated polypeptide-oligonucleotide conjugate is joined to the solid support using iEDDA click chemistry. In another embodiment, m-tetrazine rather than tetrazine is used in an iEDDA click chemistry reaction, as m-tetrazine has improved bond stability. In another embodiment, phenyl tetrazine (pTet) is used in an iEDDA click chemistry reaction.

In some embodiments, the substrate surface is functionalized with TCO, and the recording tag-labeled protein, polypeptide, peptide is immobilized to the TCO coated substrate surface via an attached m-tetrazine moiety.

In some embodiments, the generated polypeptide-oligonucleotide conjugate is joined to the solid support via its C-terminus, N-terminus, or an internal amino acid, for example, via an amine, carboxyl, or sulfhydryl group. In some embodiments, the generated polypeptide-oligonucleotide conjugate is joined to the solid support via its C-terminal amino acid of the digested polypeptide. In some embodiments, the generated polypeptide-oligonucleotide conjugate is joined to the solid support via its N-terminal amino acid of the digested polypeptide. In some embodiments, the generated polypeptide-oligonucleotide conjugate is joined to the solid support via an internal amino acid of the digested polypeptide.

In some embodiments, the generated polypeptide-oligonucleotide conjugate is joined to the solid support using a CNBr-activated, NHS-activated, aldehyde-activated, azlactone-activated, or CDI-activated support. Standard activated supports used in carboxyl coupling include carbodiimide-activated carboxyl moieties coupling to amine supports. Cysteine coupling can employ maleimide, idoacetyl, and pyridyl disulfide activated supports. In some embodiments, the generated polypeptide-oligonucleotide conjugate is joined to the solid support via carboxy terminal immobilization. In some instances, a catalytically inert derivative of trypsin (e.g., anhydrotrypsin) is used.

In some embodiments, the generated polypeptide-oligonucleotide conjugate is joined to the solid support via covalent attachment of a solid surface bound linker to a lysine group of the digested polypeptide. In some embodiments, the generated polypeptide-oligonucleotide conjugate is joined to the solid support that is passivated (e.g., blocked). In some embodiments, the generated polypeptide-oligonucleotide conjugate is joined to a bead via a thiol (e.g., maleimide reaction). In some embodiments, a plurality of generated polypeptide-oligonucleotide conjugate is joined to the solid support with suitable spacing between the polypeptide-oligonucleotide conjugates. In some instances, the density of functional coupling groups (e.g., TCO) is titrated on the surface. In some embodiments, polypeptide-oligonucleotide conjugates are spaced apart on the surface or within the volume (e.g., porous supports) of a surface at a distance of about 50 nm to about 500 nm, or a subrange thereof, e.g., or about 50 nm to about 400 nm, or about 50 nm to about 300 nm, or about 50 nm to about 200 nm, or about 50 nm to about 100 nm. In some embodiments, multiple polypeptides are spaced apart on the surface of a solid support with an average distance of at least 50 nm, at least 60 nm, at least 70 nm, at least 80 nm, at least 90 nm, at least 100 nm, at least 150 nm, at least 200 nm, at least 250 nm, at least 300 nm, at least 350 nm, at least 400 nm, at least 450 nm, or at least 500 nm. In some embodiments, polypeptide-oligonucleotide conjugates are spaced apart on the surface with an average distance of at least 50 nm. In some embodiments, polypeptide-oligonucleotide conjugates are spaced apart on the surface or within the volume of a solid support such that, empirically, the relative frequency of inter-to intra-molecular events is <1:10; <1:100; <1:1,000; or <1:10,000.

In some embodiments, the generated polypeptide-oligonucleotide conjugate is joined to the solid support that is blocked using a crowding agent. In some embodiments, the crowding agent is PEG. In some embodiments, generated polypeptide-oligonucleotide conjugates are deposited or immobilized on a substrate at any suitable density for downstream detection.

An exemplary scheme for preparing a polypeptide-oligonucleotide conjugate that is then immobilized on a surface (e.g., a bead) is provided below in scheme 1.

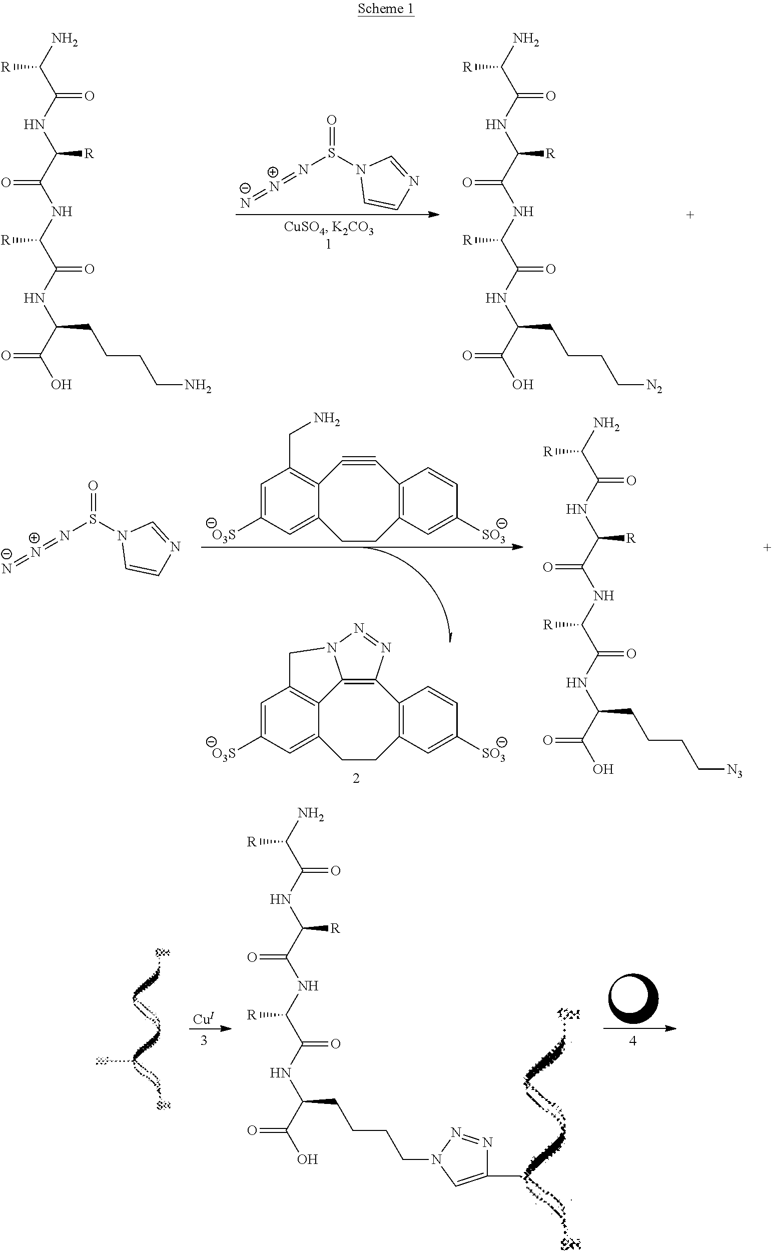

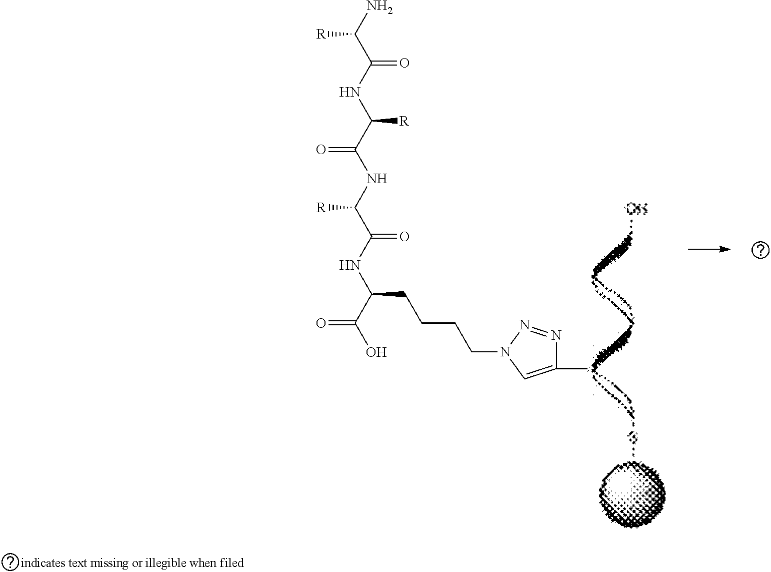

Step 1: Diazotransfer reaction. A primary amine on lysine in a peptide is converted into an azide by contacting the peptide with imidazole-1-sulfonyl in the presence of a copper salt (CuSO₄) and a base (K₂CO₃).
Step 2: Unreacted azide reagent is quenched. Unreacted imidazole-1-sulfonyl is quenched by contacting any unreacted imidazole-1-sulfonyl with a constrained cyclooctyne, resulting in strain-promoted alkyne-azide cycloaddition (SPAAC). This step is conducted in the absence of a copper catalyst.
Step 3: Conjugation of peptide to oligonucleotide. An alkyne-functionalized oligonucleotide is conjugated to the peptide through a copper-catalyzed reaction. The conjugation proceeds via a copper-catalyzed alkyne-azide cycloaddition (CuAAC). The oligonucleotide can be functionalized to contain an alkyne through modification of the oligonucleotide with 5-octadinyl dU.
Step 4: Immobilization of polypeptide-oligonucleotide conjugate to a surface. The polypeptide-oligonucleotide conjugate is immobilized to the surface of a bead, for example, by Michael-type addition of a thiol to a maleimide group.

Examples of methods for attaching barcodes to macromolecules are described in WO2023/086847 A1, which is herein incorporated by reference in its entirety.

### F. Multiple Analytes

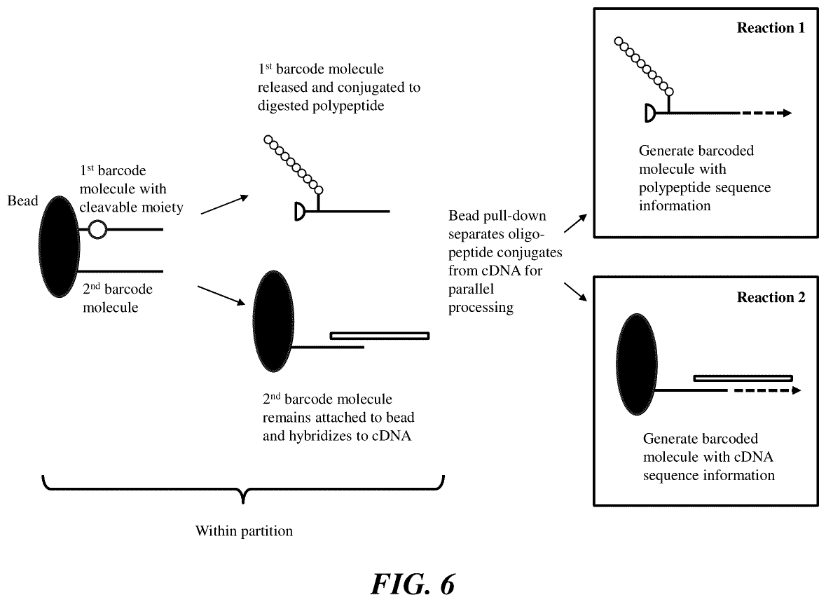

In some aspects, provided herein are methods and compositions for analysis of different species of analytes (e.g., multiomics analysis). In some embodiments, the methods and compositions facilitate parallel processing and analysis of different analytes, such as digested polypeptides and nucleic acids from the same single cells (e.g., as depicted in FIG. 6). In some embodiments, different species of nucleic acid barcode molecules provided on the same particle (e.g., bead) can be used for parallel analysis of different species of analytes (e.g., polypeptides and nucleic acids), such as from the same cell. In some embodiments, the different species of nucleic acid barcode molecules are configured to specifically couple to the different species of analytes, and to allow the different species to be separated from one another for downstream reactions and analysis (e.g., sequencing). In some embodiments, the different species of nucleic acid barcode molecules coupled to the different species of analytes are processed (e.g., partitioned, conjugated) as described in Section II and detected as described in Section III.

In some embodiments, a single particle (e.g., bead) is coupled to a first nucleic acid barcode molecule for analysis of a first analyte species (e.g., polypeptide) and a second nucleic acid barcode molecule for analysis of a second analyte species (e.g., nucleic acid). In some instances, the second analyte species is a nucleic acid derived from the cell. In some instances, the second analyte species is an endogenous nucleic acid from the cell or a derivative thereof. In some embodiments, the first nucleic acid barcode molecule is releasably coupled to the particle via a cleavable moiety, whereas the second nucleic acid barcode molecule is coupled to the particle without the cleavable moiety. This can facilitate selective release of the first nucleic acid barcode molecule via cleavage of the cleavable moiety. After coupling of the first nucleic acid barcode molecule to a first analyte (e.g., conjugation to a digested polypeptide, as described in Section II.E) and coupling of the second nucleic acid barcode molecule to a second analyte (e.g., hybridization to a nucleic acid), the first and second analyte can be separated by virtue of the fact that the first analyte is not coupled to the particle, whereas the second nucleic acid is coupled to the particle via the second nucleic acid barcode molecule. This allows for separation of the different analytes via any suitable method that isolates particle-bound analytes from non-particle-bound analytes, such as bead pull-down by gravity (e.g., centrifugation) or magnetic separation (e.g., for magnetic particles, such as magnetic beads).

The separation of different species of analytes by the provided methods and compositions has various advantages. For example, it may be desirable to subject different kinds of analytes from the same cell to different downstream reactions for generating barcoded nucleic acid molecules; in some embodiments, polypeptide-oligonucleotide conjugates can undergo a reaction for adding coding tags corresponding to amino acids to a first nucleic acid barcode molecule (e.g., as described elsewhere herein), whereas nucleic acid analytes hybridized to second nucleic acid barcode molecules can undergo a different reaction for incorporating a sequence of the nucleic acid (e.g., via an extension reaction). Furthermore, although analytes are separated for downstream processing, the different analytes can still be traced back to the same partition and/or cell of origin (e.g., by virtue of a partition-specific barcode common to the first and second nucleic acid barcode molecules), thus allowing single-cell analysis. For example, in some embodiments, the first nucleic acid barcode molecule and the second nucleic acid barcode molecule each comprise a partition-specific barcode sequence.

In some embodiments, the first analyte and second analyte are different species of analytes. The first and second analytes can be selected from any suitable species of analytes. The first and/or second analyte can be a polypeptide (e.g., a digested polypeptide as described herein) or a nucleic acid (e.g., a cDNA, a fragment of genomic DNA, or a nucleic acid probe or product thereof, such as a ligation product). In some embodiments, the first analyte is a polypeptide and the second analyte is a nucleic acid. In some embodiments, the first and second analyte are from the same cell. In some embodiments, the first analyte is a polypeptide and the second analyte is a nucleic acid associated with an mRNA from the same cell. In some embodiments, the first analyte is a polypeptide and the second analyte is a cDNA generated from an mRNA from the same cell.

While certain embodiments of the methods and compositions provided herein include analysis of a polypeptide and a nucleic acid, in some embodiments, the first analyte is a first species of nucleic acid and the second analyte is a second species of nucleic acid. In such embodiments, the first nucleic acid barcode molecule and second nucleic acid barcode molecule can be configured to hybridize specifically to the first species and the second species, respectively. For example, the first nucleic acid barcode molecule and second nucleic acid barcode molecule can comprise different capture sequences that hybridize to nucleic acids of the first species and the second species, respectively, thereby allowing isolation and parallel processing in separate reactions as described herein.

In some embodiments, the nucleic acid hybridizes directly to a nucleic acid barcode molecule (e.g., the second nucleic acid barcode molecule). In some embodiments, generating a barcoded nucleic acid molecule (e.g., second barcoded nucleic acid molecule) comprises an extension reaction (e.g., extending the nucleic acid barcode molecule using the nucleic acid as template; and/or vice versa). In some embodiments, the nucleic acid hybridizes indirectly to a nucleic acid barcode molecule (e.g., second nucleic acid barcode molecule). For example, in some embodiments, the nucleic acid hybridizes to a splint molecule, which in turn hybridizes to the second nucleic acid barcode molecule. In such embodiments, the nucleic acid can be ligated to the second nucleic acid barcode molecule to generate a barcoded nucleic acid molecule (e.g., second barcoded nucleic acid molecule), for example using the splint as template.

In some embodiments, the nucleic acid comprises DNA (e.g., cDNA or gDNA). In some embodiments, the nucleic acid can remain hybridized to the second nucleic acid barcode molecule coupled to the particle during processing steps for conjugating the digested polypeptide to the first nucleic acid barcode molecule. In some embodiments, providing the nucleic acid as DNA (e.g., as opposed to RNA) provides improved stability during the conjugating steps.

In some embodiments, the nucleic acid is a nucleic acid endogenous to the cell, or a product thereof. In some embodiments, the nucleic acid is a product of an endogenous nucleic acid (e.g., cDNA). In some embodiments, the nucleic acid not endogenous to the cell (e.g., a probe or a product thereof, such as a ligation product of a probe specific for an mRNA in the cell). In some embodiments, the method comprises generating the nucleic acid. In some embodiments, the method comprises generating the nucleic acid in the cell. In some embodiments, the nucleic acid is generated prior to partitioning and lysis of the cell for release of the nucleic acid and hybridization to the second nucleic acid barcode molecule. In some embodiments, the nucleic acid can be generated after the partitioning and/or lysis. In some embodiments, the nucleic acid comprises a sequence that hybridizes to a sequence present on the second nucleic acid barcode molecule (e.g., capture sequence).

In some embodiments, the nucleic acid comprises cDNA. In some embodiments, the method comprises performing a reverse transcription reaction to generate the cDNA. In some embodiments, the cDNA is generated in a reverse transcription reaction. In some embodiments, the reverse transcription is performed in the cell prior to providing (e.g., generating) the partition. In some embodiments, the reverse transcription is performed in the cell prior to partitioning and lysis. Any suitable reaction for generating a cDNA capable of coupling, such as hybridizing directly or indirectly (e.g., via a splint), to the second nucleic acid barcode molecule may be used. In some embodiments, the reverse transcription reaction comprises extending a reverse transcription primer (RT primer) that hybridizes to the RNA. In some embodiments, the RT primer comprises a hybridization region for hybridizing to the RNA. The RT primer hybridization region can comprise a polyT sequence (e.g., for hybridizing to mRNA polyA tails), or a sequence for hybridizing to particular species of analytes (e.g., immune receptor constant region-encoding sequences). In some embodiments, the RT primer comprises an overhang, such as a 5′ overhang sequence that is not complementary to the RNA. The overhang can comprise a sequence for hybridizing the cDNA to the second nucleic acid barcode molecule in downstream steps (e.g., directly or indirectly via a splint molecule). In some embodiments, the reverse transcription reaction is performed using a reverse transcriptase that incorporates a non-templated 3′ terminal sequence. In some embodiments, the 3′ terminal sequence of the cDNA hybridizes to the second nucleic acid barcode molecule in downstream steps. In some embodiments, the 3′ terminal sequence hybridizes to a template switch oligonucleotide (TSO) and the reverse transcription reaction further includes incorporating a sequence complementary to the TSO into the cDNA. In some embodiments, the sequence complementary to the TSO is captured by (e.g., hybridizes to) the second nucleic acid barcode molecule in downstream steps.

In some embodiments, the nucleic acid is a probe that has been contacted with the cell. In some embodiments, the nucleic acid is a product of the probe, such as a ligation product (e.g., a ligated probe). In some embodiments, the nucleic acid is a ligation product of a probe bound directly or indirectly to an analyte in the cell. In some embodiments, the analyte comprises RNA and the ligation product is a product of RNA-templated ligation. In some embodiments, the nucleic acid is a nucleic acid probe. In some embodiments, the nucleic acid probe is a ligated probe, e.g., a ligation product of a probe. In some embodiments, the probe is ligated by RNA templated ligation. In some embodiments, the probe comprises two probe molecules that hybridize to a target RNA molecule in the cell, wherein the two probe molecules are ligated using the target RNA molecule as template. In some embodiments, the cell is washed to remove unligated probe molecules, while the ligated probe remains hybridized to the target RNA molecule, for example due to increased melting temperature after ligation. In some embodiments, the nucleic acid probe is ligated in the cell prior to partitioning and lysis of the cell for release of the nucleic acid probe and hybridization to the second nucleic acid barcode molecule. In some embodiments, the ligated probe comprises a sequence that hybridizes to the second nucleic acid barcode molecule. The sequence that hybridizes to the second nucleic acid barcode molecule can be present in an overhang of the probe which does not hybridize to the RNA molecule. Instead of an RNA molecule, the probe can bind and/or hybridize to any suitable nucleic acid present in the cell, such as to an oligonucleotide coupled to an analyte-specific antibody. Various probes suitable for being used as the nucleic acid in accordance with the methods and compositions provided herein are described, for example, in International Patent Application Publication Nos. WO2024/006392, WO2019/165318 and WO2021/041974, U.S. Patent Application Publication Nos. 2020/0239874, and U.S. Pat. No. 11,639,928, which are herein entirely incorporated by reference for all purposes.

In some embodiments, the nucleic acid comprises a fragment of genomic DNA. In some embodiments, the fragment of genomic DNA is generated using a transposase enzyme. In some embodiments, the fragment of genomic DNA is generated via tagmentation. In some embodiments, the nucleic acid comprises genomic DNA (gDNA), such as a gDNA fragment. The gDNA fragment can be generated by any suitable method. In some embodiments, the gDNA fragment comprises one or more flanking sequences that flank an endogenous sequence of gDNA. The one or more flanking sequences can comprise any suitable sequence(s), including barcodes, amplification sites, and adapters (e.g., sequencing adapters). In some embodiments, the gDNA fragment is generated in a workflow for assessing chromatin accessibility. Examples of workflows for generating gDNA fragments can include methods for fragmenting gDNA and adding nucleic acid adapters, such as tagmentation, CUT&TAG, CUT&RUN, and ATAC-seq. In some embodiments, the gDNA fragment is generated using an enzyme. In some embodiments, the gDNA fragment is generated using a transposase enzyme (e.g., a Tn5 transposase). In some embodiments, the gDNA fragment is generated using an MNase enzyme. In some embodiments, the gDNA fragment is generated in a tagmentation reaction. In some embodiments, the gDNA fragment is generated using a workflow including an Assay for Transposase-Accessible Chromatin using sequencing (ATAC-seq) step, such as a single-cell ATAC-seq step. Any suitable method may be used for generating gDNA fragments for analysis in accordance with the methods and compositions provided herein.

Provided herein is a method comprising providing a partition comprising: i) a digested polypeptide derived from a cell, ii) a nucleic acid derived from the cell, and iii) a particle coupled to a first nucleic acid barcode molecule and a second nucleic acid barcode molecule; selectively releasing the first nucleic acid barcode molecule from the particle in the partition, wherein the second nucleic acid barcode molecule remains coupled to the particle in the partition; generating a polypeptide-oligonucleotide conjugate by coupling the digested polypeptide to the first nucleic acid barcode molecule in the partition; hybridizing the nucleic acid directly or indirectly to the second nucleic acid barcode molecule in the partition, thereby forming a particle complex comprising the particle, the second nucleic acid barcode molecule, and the nucleic acid; separating the polypeptide-oligonucleotide conjugate from the particle complex into a first vessel and a second vessel, respectively; and generating a barcoded nucleic acid molecule using the nucleic acid and the second nucleic acid barcode molecule, wherein the barcoded nucleic acid molecule comprises: 1) a sequence of the second nucleic acid barcode molecule or a complement thereof, and 2) a sequence of the nucleic acid or a complement thereof.

In some aspects, a method provided herein comprises providing a partition comprising: i) a digested polypeptide derived from a cell, ii) a nucleic acid derived from the cell, and iii) a particle coupled to a first nucleic acid barcode molecule and a second nucleic acid barcode molecule. In some embodiments, the method comprises selectively releasing the first nucleic acid barcode molecule from the particle in the partition, wherein the second nucleic acid barcode molecule remains coupled to the particle in the partition. The first nucleic acid barcode molecule can be selectively released upon formation of the partition, or in any suitable subsequent step, such as after the provision of a suitable stimulus and/or processing step. In certain embodiments, the particle coupled to the first nucleic acid barcode molecule and second nucleic acid barcode molecule is provided in the partition, and the first nucleic acid molecule is released from the particle in the partition prior to the cell (and/or digested polypeptide and nucleic acid therefrom) being added to the partition. Similarly, in some embodiments, the first nucleic acid barcode molecule can be released from the particle in the partition prior to generating the digested polypeptide (e.g., by digestion of a protein released from the cell). Thus, in some embodiments, the method comprises providing a partition comprising: i) a digested polypeptide derived from a cell, ii) a nucleic acid derived from the cell, and iii) a particle associated with a first nucleic acid barcode molecule that has been released from the particle and a second nucleic acid barcode molecule that is coupled to the particle. In some instances, the nucleic acid derived from the cell comprises is a product of an endogenous nucleic acid (e.g., cDNA).

In some embodiments, the method comprises generating a polypeptide-oligonucleotide conjugate by conjugating the digested polypeptide to the first nucleic acid barcode molecule in the partition. The polypeptide-oligonucleotide conjugate can be generated by any suitable means, such as any of those provided herein.

In some embodiments, the method comprises hybridizing the nucleic acid directly or indirectly to the second nucleic acid barcode molecule in the partition, thereby forming a particle complex comprising the particle, the second nucleic acid barcode molecule, and the nucleic acid. In some embodiments, the method comprises hybridizing the nucleic acid to the second nucleic acid barcode molecule in the partition, thereby forming a particle complex comprising the particle, the second nucleic acid barcode molecule, and the nucleic acid. In some embodiments, the method comprises hybridizing the nucleic acid to an intermediate molecule (e.g., a splint molecule), wherein the intermediate molecule also hybridizes to the second nucleic acid barcode molecule in the partition, thereby forming a particle complex comprising the particle, the second nucleic acid barcode molecule, the intermediate molecule, and the nucleic acid. In some embodiments, the splint molecule comprises locked nucleic acid (LNA).

In some embodiments, the method comprises separating the polypeptide-oligonucleotide conjugate from the particle complex. In some embodiments, the method comprises separating the polypeptide-oligonucleotide conjugate from the particle complex into a first vessel and a second vessel, respectively. In some embodiments, the method comprises generating a first barcoded nucleic acid molecule using the polypeptide-oligonucleotide conjugate. In some embodiments, the first barcoded nucleic acid molecule comprises: 1) a sequence of the first nucleic acid barcode molecule or a complement thereof, and 2) a nucleic acid sequence corresponding to an amino acid sequence of the digested polypeptide. In some embodiments, the method comprises generating a second barcoded nucleic acid molecule using the nucleic acid and the second nucleic acid barcode molecule. In some embodiments, the second barcoded nucleic acid molecule comprises: 1) a sequence of the second nucleic acid barcode molecule or a complement thereof, and 2) a sequence of the nucleic acid or a complement thereof. In some embodiments, the first barcoded nucleic acid molecule and the second barcoded nucleic acid molecule are detected and/or analyzed as described in Section III.

In some embodiments, the method further comprises sequencing the first barcoded nucleic acid molecule or a derivative thereof and the second barcoded nucleic acid molecule or a derivative thereof. In some embodiments, the method comprises using results of the sequencing to determine the presence, identity, sequence, and/or abundance of the digested polypeptide and the nucleic acid derived from the cell. In some embodiments, the analysis can be used to analyze endogenous cellular analytes from which the digested polypeptide and nucleic acid are derived.

In some embodiments, the first nucleic acid barcode molecule is coupled to the particle via a cleavable moiety. In some embodiments, the second nucleic acid barcode molecule is not coupled to the particle via the cleavable moiety. The cleavable moiety can be any moiety suitable for releasably coupling the first nucleic acid barcode molecule to the particle (e.g., bead), such that the nucleic acid barcode molecule can be released from the particle upon and/or after providing (e.g., generating) the partition. In some embodiments, selectively releasing the first nucleic acid barcode molecule from the particle in the partition comprises cleaving the cleavable moiety. In some embodiments, the cleavable moiety comprises a disulfide bond.

In some embodiments, separating the polypeptide-oligonucleotide conjugate from the particle complex comprises centrifugation. In some embodiments, the particle is magnetic. In some embodiments, separating the polypeptide-oligonucleotide conjugate from the particle complex comprises magnetic separation.

In some embodiments, the first nucleic acid barcode molecule and the second nucleic acid barcode molecule each comprise a partition-specific barcode sequence. In some embodiments, the first barcoded nucleic acid molecule comprises the partition-specific barcode sequence or a complement thereof, and the second barcoded nucleic acid molecule comprises the partition-specific barcode sequence or a complement thereof. In some embodiments, the first nucleic acid barcode molecule and second nucleic acid barcode molecule comprise Unique Molecular Identifier (UMI) sequences. In some embodiments, the first nucleic acid barcode molecule and second nucleic acid barcode molecule each comprise one or more functional sequences.

In some embodiments, the partition is a partition among a plurality of partitions. In some embodiments, the partition is a droplet among a plurality of droplets. In some embodiments, the partition is a well among a plurality of wells. In some embodiments, the particle is a bead. In some embodiments, the method comprises lysing the cell in the partition. In some embodiments, lysing the cell releases nucleic acid and proteins from the cell. In some embodiments, the digested polypeptide is a digestion product of a protein from the cell. Thus, in some embodiments, the digested polypeptide can be generated in the partition after cellular lysis.

In some embodiments, the partition is a partition among a plurality of partitions (e.g., as described in Section II.B). In some aspects, a large number of single cells in individual partitions of the plurality of partitions can be analyzed in parallel, e.g., in a single-cell sequencing workflow. Thus, in some embodiments, the methods and compositions provided herein facilitate large-scale single-cell parallel processing and/or analysis of different analyte species, such as protein/polypeptide and nucleic acid (e.g., mRNA) expression.

### III. Detection and Analysis

In some embodiments, the method comprises determining (e.g., detecting) the sequence of the digested polypeptide and the sequence of the nucleic acid barcode molecule of the polypeptide-oligonucleotide conjugate or a derivative thereof. In some embodiments, the method comprises determining (e.g., detecting) the sequence of the first barcoded nucleic acid molecule and the second barcoded nucleic acid molecule generated as described in Section II.F.

In some instances, to determine the sequence of the digested polypeptide of the polypeptide-oligonucleotide conjugate generated as described in Section II, the digested polypeptide of the polypeptide-oligonucleotide conjugate is contacted with a binding agent comprising a coding tag sequence associated with the binding agent. In some instances, the nucleic acid barcode molecule of the polypeptide-oligonucleotide conjugate is extended with the coding tag sequence associated with the binding agent or a complement thereof, thereby generating an extended nucleic acid barcode molecule. In some instances, the binding agent is specific to a terminal amino acid or terminal amino acid derivative from the digested polypeptide. In some instances, the binding agent binds to at least a terminal amino acid or terminal amino acid derivative from the digested polypeptide. In some instances, the binding agent is specific to the N-terminal amino acid residue of the digested polypeptide or a derivative thereof. In some instances, additional binding agents are used and the extended nucleic acid barcode molecule is further extended with one or more additional coding tag sequences associated with the additional binding agents. In some instances, the extended nucleic acid barcode molecule identifies the digested polypeptide and the single cell where the digested polypeptide originated. In some instances, determining the sequence of the nucleic acid barcode molecule comprises sequencing the nucleic acid barcode molecule or a derivative thereof. In some instances, an extended nucleic acid barcode molecule or a derivative thereof comprising (i) the coding tag sequence or a complement thereof and (ii) a sequence of the nucleic acid barcode or a complement thereof is sequenced.

In some embodiments, a plurality of polypeptide-oligonucleotide conjugates spread on a substrate is detected by imaging. In some embodiments, a plurality of signals associated with the digested polypeptide (e.g., particular amino acid(s)) and/or sequence(s) of the nucleic acid barcode molecule) are detected at a location on the substrate by imaging the polypeptide-oligonucleotide conjugates spread on the substrate. Polypeptide detection and nucleic acid barcode (e.g., CBC sequence) detection on the substrate can be performed in either order. In some examples, signals associated with the polypeptide is detected after the sequence of the nucleic acid barcode molecule or derivative thereof is detected. In some examples, signals associated with the polypeptide is detected prior to detecting the sequence of the nucleic acid barcode molecule or derivative thereof.

In some embodiments, a plurality of digested polypeptides from a single cell are in a partition, wherein the partition comprises a plurality of nucleic acid barcode molecules unique to the partition and the contents of the partition are contacted with a reducing agent and a conjugation reagent to generate a plurality of polypeptide-oligonucleotide conjugates by coupling the plurality of digested polypeptides to the plurality of nucleic acid barcode molecules. In some instances, polypeptide-oligonucleotide conjugates are pooled from a plurality of partitions (e.g., droplets or wells) and dispersed on a substrate (e.g., a glass surface such as a slide). In some instances, a plurality of signals associated with the digested polypeptide (e.g., particular amino acid(s)) and the nucleic acid barcode molecule are detected at a location on the substrate by imaging the conjugates spread on the substrate.

In some embodiments, the nucleic acid barcode molecules of the polypeptide-oligonucleotide conjugates or extensions thereof are further processed e.g., to amplify the molecules and/or append sequencing platform specific sequences. See, e.g., U.S. Pat. Pub. 2018/0105808, which is hereby entirely incorporated by reference for all purposes. In some embodiments, the nucleic acid barcode molecules of the polypeptide-oligonucleotide conjugates or extensions or derivatives generated therefrom are sequenced on a suitable sequencing platform.

### A. Polypeptide Detection

In some embodiments, the sequence of amino acids in the digested polypeptide or a portion thereof of the polypeptide-oligonucleotide conjugate generated (e.g., as described in Section II) is determined. In some embodiments, the amino acids in the digested polypeptide are determined using binding agents that recognizes at least one of the amino acids of the polypeptides. In some instances, the binding agent binds to a terminal amino acid of the digested polypeptide or a derivative thereof. In some embodiments, the nucleic acid barcode molecule conjugated to the digested polypeptide has a free 3′ end for extension (e.g., by ligation or by polymerase). In some embodiments, the free 3′ end is available to incorporate a sequence associated with the sequence of amino acids in the digested polypeptide. In some instances, a binding agent comprises a coding tag sequence associated with the binding agent (e.g., corresponding to the moiety bound by the binding agent). In some instances, the coding tag sequence of the binding agent is transferred by ligation or polymerase extension to the nucleic acid barcode molecule to generate an extended nucleic acid barcode molecule. In some embodiments, multiple cycles of the binding agent contacting and transfer of the coding tag sequence is performed and a terminal amino acid or terminal amino acid derivative is cleaved from the digested polypeptide at the end of each cycle. For example, polypeptide sequence determination in a cyclic manner is described in U.S. Patent Application Publication No. 2024/0377409 and 2025/0051756, which are entirely incorporated herein by reference for all purposes. In some aspects, N-terminal Edman degradation is compatible with nucleic acid barcoding and is used for cycles of polypeptide sequencing. In some embodiments, the nucleic acid barcode molecule conjugated to the digested polypeptides and any extensions thereof encoding the amino acids of the polypeptide are sequenced. In some embodiments, the sequence associated with the polypeptide or portion thereof allows the common cell barcode in the nucleic acid barcode molecule to associate a plurality of polypeptides that originated in the same partition.

In some embodiments, the amino acids in the digested polypeptide are determined using binding agents that recognize at least one of the amino acids of the polypeptides (e.g., an N-terminal amino acid residue) in an immobilized single-amino acid complex. In some instances, the immobilized single-amino acid complex comprises a nucleic acid barcode molecule comprising cycle information. In some cases, the amino acid residue is cleaved and separated from the polypeptide prior to being contacted by the binding agent. For example, polypeptide sequence determination in a cyclic manner is described in U.S. Patent Application Publication No. 2024/0210410 and 2024/0044909, which are hereby incorporated by reference in their entirety.

In some embodiments, the sequence of amino acids in the digested polypeptide of the polypeptide-oligonucleotide conjugate generated is determined using a nanopore, e.g., as described in U.S. Patent Application Publication No. 2024/0337660, which is hereby incorporated by reference in its entirety. In some instances, a linearized and/or expanded polypeptide chain is generated and analyzed using a nanopore. In some instances, the linearized and/or expanded polypeptide chain comprises a nucleic acid backbone. In some instances, a polypeptide chain comprises modified amino acids of the digested polypeptide and is analyzed using a nanopore. In some instances, a polypeptide chain comprises a plurality of polymerizable molecules covalently linked together. In some instances, nanopore analysis comprises translocating a modified amino acid through or adjacent to a nanopore. In some instances, the linearized and/or expanded polypeptide chain construct is assembled using a cyclic, iterative process comprising attaching a linker to an amino acid of a digested polypeptide. In some instances, the linearized and/or expanded polypeptide chain construct is assembled using a cyclic, iterative process comprising reacting a plurality of amino acids with a plurality of oligonucleotides. In some instances, the linearized and/or expanded polypeptide chain construct is assembled using ligation.

In some instances, nanopore-based sequencing of the digested polypeptide is performed using an intramolecular expansion approach as described in International Patent Application Publication No. WO2025/166050, which is hereby incorporated by reference in its entirety. In some instances, an expanded polypeptide chain construct comprises a polymerizable molecule. In some instances, the linearized and/or expanded polypeptide chain comprises a series of modified amino acids, each comprising a cleaved amino acid, a linker, and a polymerizable molecule, as described in International Patent Application Publication No. WO2025/166050, which is hereby incorporated by reference in its entirety. In some embodiments, the modified amino acids are tethered together as a stacked construct by coupling successive amino acid-linker complexes to a capture moiety. In some instances, a stacked modified amino acid is individually translocated through or adjacent to the nanopore or a nanogap sequencer, and a signal (e.g., an ionic current) is measured and used to determine the identity of each amino acid.

In some embodiments, the sequence of amino acids in the digested polypeptide of the polypeptide-oligonucleotide conjugate is used in a workflow for detecting a cleaved terminal amino acid or terminal amino acid derivative from the digested polypeptide, e.g., as described in U.S. Patent Application Publication No. 2024/0409995, which is hereby incorporated by reference in its entirety.

In some embodiments, the digested polypeptide sequence or portion thereof is determined using any suitable detection methods, such as binding agents that recognizes at least one of the amino acids of the polypeptides. In some instances, a binding agent binds to a terminal amino acid of the digested polypeptide or a derivative thereof. In some embodiments, a plurality of binding agents is used for binding to different moieties. In some embodiments, the sequence of the digested polypeptide is detected by performing fluorosequencing. e.g., as described in U.S. Patent Application Publication No. 2024/0201198, which is hereby incorporated by reference in its entirety. In some instances, determining the sequence of the digested polypeptide comprises detecting a plurality of signals associated with the digested polypeptide. In some instances, the plurality of signals is from a plurality of binding agents labeled with a detectable label.

In some cases, the terminal amino acid of the digested polypeptides on the substrate or a derivative thereof is contacted with a binding agent. In some embodiments, the binding agent is detectably labeled. In some embodiments, the binding agent comprises a fluorophore. In some instances, the terminal amino acid or a terminal amino acid derivative of the digested polypeptide is detected while in the conjugate. In some instances, the terminal amino acid or a terminal amino acid derivative of the digested polypeptide is detected after it is cleaved from the digested polypeptide.

### B. Nucleic Acid Sequence Detection

In some instances, the nucleic acid barcode molecule or a derivative and/or extension thereof comprising a sequence of the nucleic acid barcode associated with the single cell origin of the digested polypeptide is sequenced. In some embodiments, the sequence of the first barcoded nucleic acid molecule and the second barcoded nucleic acid molecule generated as described in Section II.F is determined. In some instances, the nucleic acid barcode molecule or a derivative and/or extension thereof comprises a functional sequence, a primer sequence (e.g., sequencing primer sequence or partial sequencing primer sequence). In some aspects, any of the barcoded nucleic acid molecules or derivatives thereof are further processed and analyzed by any suitable technique, including nucleic acid sequencing. In some embodiments, library construction is performed and a plurality of barcoded nucleic acid molecules are sequenced as described elsewhere herein. For example, the sequencing comprises sequencing by synthesis. In some aspects, subsequent to the sequencing, the data generated by the sequencing is analyzed (e.g., traced back to the same partition and/or cell of origin by virtue of a common barcode).

In some instances, nucleic acid barcode molecules or derivatives and/or extensions thereof are collected, pooled, and subjected to further processing (e g., reverse transcription, adapter attachment, amplification, clean up, sequencing). In some cases, the nucleic acid barcode molecules or derivatives and/or extensions thereof comprise a functional sequence, for example, for attachment to a sequencing flow cell, such as, for example, a P5 sequence (or a portion thereof) for Illumina® sequencing. In some cases, the nucleic acid barcode molecules or derivatives and/or extensions thereof comprise another functional sequence, such as, for example, a P7 sequence (or a portion thereof) for attachment to a sequencing flow cell for Illumina sequencing. In some cases, the nucleic acid barcode molecules or derivatives and/or extensions thereof comprise a barcode sequence and a unique molecular identifier (UMI). In some cases, the nucleic acid barcode molecules or derivatives and/or extensions thereof comprise an R1 primer sequence for Illumina sequencing. In some cases, the nucleic acid barcode molecules or derivatives and/or extensions thereof comprise an R2 primer sequence for Illumina sequencing. Systems and methods for sequencing are described in U.S. Patent Application Publication No. 2022/0025438, the content of which is herein incorporated by reference in its entirety.

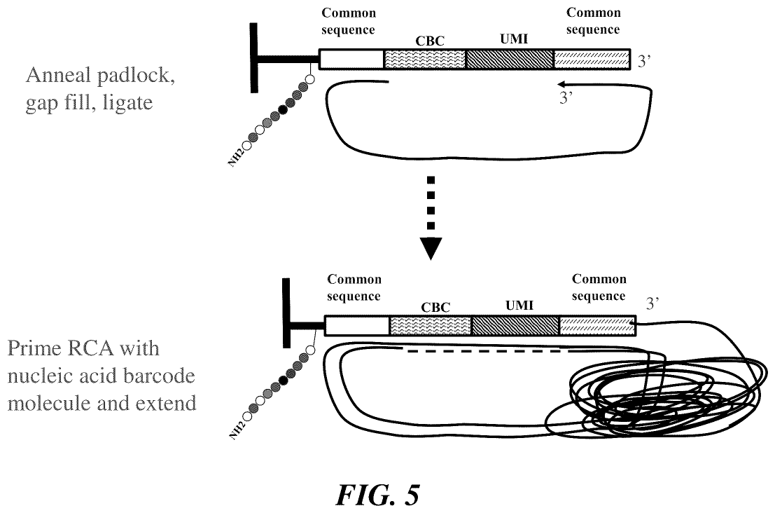

In some instances, the nucleic acid barcode molecule or a derivative comprises a cell barcode (CBC) or a complement thereof. In some embodiments, the nucleic acid barcode molecule of the generated polypeptide-oligonucleotide conjugate is detected using a circularizable probe (e.g., a padlock probe) to amplify the sequence of the nucleic acid barcode molecule. In some embodiments, rolling circle amplification (RCA) is performed by binding (e.g., hybridizing) a circularizable probe to the nucleic acid barcode molecule or an extension or derivative thereof. In some instances, the circularizable probe comprises a 5′ hybridization region and a 3′ hybridization region that binds a first common sequence and a second common sequence, respectively, in the nucleic acid barcode molecule, as shown in FIG. 5. In some embodiments, the end of the circularizable probe is gap filled using the nucleic acid barcode molecule as template. In some embodiments, different nucleic acid barcode molecules comprising different cell barcode (CBC) sequences corresponding to different single cells (and corresponding partitions) can utilize the same circularizable probe with common sequences as binding arms. In some embodiments, the extended circularizable probe with the CBC sequence or a complement thereof incorporated is ligated to generate a circularized probe. In some embodiments, the circularized probe serves as a template for RCA. In some embodiments, the RCA is performed by extending the 3′ end of the nucleic acid barcode molecule of the polypeptide-oligonucleotide conjugate as a primer. In some embodiments, the plurality of signals associated with the nucleic acid barcode molecule is detected by performing sequential hybridization of probes, sequencing by ligation, sequencing by synthesis, sequencing by binding, sequencing by avidity, or a combination thereof. In some instances, a sequence of the nucleic acid barcode molecule is detected by performing sequential hybridization of detectably-labeled probes to the RCA product. For example, a common sequencing primer is used to bind at least a portion of the generated RCA products on the substrate. In some embodiments, imaging is performed to detect a sequence of the RCA product at the location on the substrate. In some embodiments, imaging is performed to detect a sequence of the RCA product and/or a signal associated with the polypeptide sequence at the location on the substrate.

In some embodiments, the nucleic acid barcode molecule of the generated polypeptide-oligonucleotide conjugate is detected by circularizing the nucleic acid barcode molecule to generate a circularized nucleic acid barcode molecule. In some embodiments, the nucleic acid barcode molecule of the generated polypeptide-oligonucleotide conjugate is released from the substrate prior to being circularized. In some instances, the circularized nucleic acid barcode molecule comprises a CBC and one or more sequences associated with the digested polypeptide. In some embodiments, the circularized nucleic acid barcode molecule is an extension product of the nucleic acid barcode molecule conjugated to the digested polypeptide. In some instances, the circularized nucleic acid barcode molecule is used as a template for an amplification reaction. In some embodiments, the circularized nucleic acid barcode molecule is used as a template for RCA. In some embodiments, the RCA is performed by binding a primer to the circularized nucleic acid barcode molecule and extending the primer using the circularized nucleic acid barcode molecule as template. In some embodiments, a plurality of signals associated with the circularized nucleic acid barcode molecule is detected by performing sequential hybridization of probes, sequencing by ligation, sequencing by synthesis, sequencing by binding, sequencing by avidity, or a combination thereof. For example, imaging is performed to detect a sequence of the RCA product at the location on the substrate. In some embodiments, imaging is performed to detect a sequence of the RCA product and/or a signal associated with the polypeptide sequence at the location on the substrate.

The polypeptide detection and nucleic acid barcode (e.g., CBC sequence) detection on the substrate can be performed in either order. In some cases, signals associated with the polypeptide is detected prior to detecting the nucleic acid barcode sequence or complement thereof. In some examples, signals associated with the polypeptide is detected after the nucleic acid barcode sequence is detected.

In some embodiments, determining the sequence of the nucleic acid barcode molecule of the polypeptide-oligonucleotide conjugate or a derivative thereof is performed using a circularizable probe. In some instances, the circularizable probe is a padlock probe. In some embodiments, a circularizable probe comprises a linear oligonucleotide sequence that, upon hybridization to the nucleic acid barcode molecule, forms a probe that can be circularized. In some instances, a probe is circularized via ligation using the nucleic acid barcode molecule as a template. In some instances, a probe is circularized via primer extension followed by ligation using the nucleic acid barcode molecule as a template. For example, a plurality of circularizable probes hybridizes to different nucleic acid barcode molecules comprising different cell barcode sequences (e.g., associated with different single cells and/or different partitions). In some instances, a gap is present between the ends of the circularizable probe upon hybridization to a nucleic acid barcode molecule. In some instances, the gap is formed flanking the cell barcode of the nucleic acid barcode molecule. In some instances, the gap is filled using the nucleic acid barcode molecule such that the gap filled circularizable probe is extended to incorporate the cell barcode sequence or a complement thereof.

In some aspects, a circularizable probe of the plurality of circularizable probes comprises a 5′ hybridization region and a 3′ hybridization region that binds a first common sequence and a second common sequence, respectively, in the nucleic acid barcode molecule. In some instances, once hybridized to the complementary sequences in the nucleic acid barcode molecule, the first and second hybridization sequences are positioned with a gap of about at least 5, 10, 15 or more nucleotides.

In some embodiments, a gap of a circularizable probe hybridized to the nucleic acid barcode molecule is filled by extending a 3′ end of the circularizable probe. In some embodiments, a polymerase is used to extend the 3′ end using the nucleic acid barcode molecule as a template, thereby filling the gap using nucleotide sequence in the nucleic acid barcode molecule. In some embodiments, gap filling by the polymerase incorporates nucleotides residues into the circularizable probe, and the incorporated nucleotide sequence is complementary to a cell barcode sequence in the nucleic acid barcode molecule. In some embodiments, gap filling by the polymerase incorporates nucleotides residues into the circularizable probe, and the incorporated nucleotide sequence is complementary to a UMI in the nucleic acid barcode molecule. In some embodiments, gap filling by the polymerase incorporates nucleotides residues into the circularizable probe, and the incorporated nucleotide sequence is complementary to a cell barcode sequence and a UMI in the nucleic acid barcode molecule. In some embodiments, a 3′ end and a 5′ end of the circularizable probe is ligated using the nucleic acid barcode molecule as a template after gap filling. In some embodiments, the ligation of the 3′ end and the 5′ end is preceded by gap filling.

In some embodiments, a gap of at least 1, 2, 3, 4, 5,10, 12, 15, 20 or more nucleotides between the 5′ hybridization region and a 3′ hybridization region of the circularizable probe hybridized to a nucleic acid barcode molecule is filled prior to ligation. In some embodiments, a gap of 1-50 nucleotides, 1-40 nucleotides, 1-30 nucleotides, 1-20 nucleotides, 1-10 nucleotides, 1-5 nucleotides, 2-50 nucleotides, 2-40 nucleotides, 2-30 nucleotides, 2-20 nucleotides, 2-10 nucleotides, 2-5 nucleotides, 5-50 nucleotides, 5-40 nucleotides, 5-30 nucleotides, 5-20 nucleotides, 5-10 nucleotides, 10-50 nucleotides, 10-40 nucleotides, 10-30 nucleotides, or 10-20 nucleotides between the hybridization regions hybridized to a nucleic acid barcode molecule is filled prior to ligation. In specific embodiments, the gap may be a gap of about 1, 2, 3, 4, 5, 6, 7, 8, 9 or 10 or more nucleotides, of any integer (or range of integers) of nucleotides in between the indicated values herein. In some embodiments, a gap of no more than 20, no more than 30, no more than 40, or no more than 50 nucleotides is filled. In some instances, the gap is a gap of 1 to 60 nucleotides or a gap of 1 to 40 nucleotides or a gap of 3 to 40 nucleotides.

In some embodiments, performing the gap-fill reaction comprises using a gap-fill polymerase (e.g., DNA polymerase) to extend an end of the circularizable probe using the nucleic acid barcode molecule as a template as shown in FIG. 5.

In some embodiments, the gap is filled using a polymerase that has no or little strand displacement activity. In some embodiments, the gap is filled using a gap-fill polymerase that incorporates one or more deoxyribonucleotide residues and/or one or more ribonucleotide residues into a 3′ end of the circularizable probe. In some instances, the gap filling is performed using a polymerase (e.g., DNA polymerase) in the presence of appropriate dNTPs and other cofactors, under isothermal conditions or non-isothermal conditions. Suitable DNA polymerases include but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes. In some instances, the gap filling is performed using a Sulfolobus DNA Polymerase. In some instances, the gap filling is performed using a Sulfolobus DNA Polymerase IV. In some instances, the gap filling is performed using a Klenow fragment. In some instances, the gap filling is performed using a Taq DNA polymerase or a modification or fragment thereof. In some instances, the gap filling is performed using a Hemo KlenTaq® DNA polymerase.

In some instances, the gap filling is performed using a DNA polymerase capable of incorporating at least about 1, at least about 2, at least about 3, at least about 4, at least about 5, at least about 6, at least about 7, at least about 8, at least about 9, at least about 10, at least about 15, at least about 20, at least about 25, at least about 30, at least about 40, or at least about 50 nucleotides in a single binding event before dissociating from the nucleic acid barcode molecule.

In some aspects, after gap filling using the nucleic acid barcode molecule, the method further comprises one or more steps such as ligation, extension and/or amplification of the plurality of circularizable probes hybridized to the nucleic acid barcode molecules. In some instances, a circularizable probe hybridized to the nucleic acid barcode molecule to generate a product (e.g., comprising a sequence associated with the partition as described in Section II). In some aspects, the product is generated using RCA. In some embodiments, the method comprises ligating the ends of a circularizable probe hybridized to the nucleic acid barcode molecule to form a circularized probe. In some embodiments, the method comprises ligating the ends of a plurality of circularizable probes hybridized to the nucleic acid barcode molecule to form a plurality of circularized probes. In some instances, the method further comprises generating a plurality of rolling circle amplification products using the plurality of circularizable probes.

In some embodiments, the RCA comprises a linear RCA. In some embodiments, the RCA comprises a branched RCA. In some embodiments, the RCA comprises a dendritic RCA. In some embodiments, the RCA comprises any combination of the foregoing. In some embodiments, a plurality of ligated circularizable probes are detected. In some cases, RCA is not performed.

In some embodiments, the circular construct (e.g., circularized probe) is formed using ligation. In some embodiments, the ligated probe is generated using the nucleic acid barcode molecule as template. In some embodiments, the ligation is a DNA-DNA templated ligation. In some embodiments, the ligation comprises enzymatic ligation, chemical ligation, template dependent ligation, and/or template independent ligation. In some embodiments, the enzymatic ligation involves use of a ligase (e.g., an RNA ligase, a DNA ligase). Ligases comprise ATP-dependent double-strand polynucleotide ligases, NAD-i-dependent double-strand DNA or RNA ligases and single-strand polynucleotide ligases, for example any one of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° NTM DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In any one of the embodiments herein, the ligation comprises using a ligase selected from the group consisting of a Chlorella virus DNA ligase (PBCV DNA ligase), a T4 RNA ligase, a T4 DNA ligase, and a single-stranded DNA (ssDNA) ligase. In some embodiments, the ligation comprises using a PBCV-1 DNA ligase or variant or derivative thereof and/or a T4 RNA ligase 2 (T4 Rnl2) or variant or derivative thereof. In some embodiments, the ligase is a T4 RNA ligase. In some embodiments, the ligase comprises a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, a ligation buffer disclosed herein comprises ATP and/or NAD+ as an alternative adenylation donor for nucleic acid ends ligation catalyzed by a ligase.

Following formation of the plurality of circularized probes, in some instances, an amplification primer is added. In other instances, a separate amplification primer is not added. In some embodiments, an amplification primer is not needed and an extension reaction uses the nucleic acid barcode molecule as a primer. In some embodiments, a washing step is performed to remove any unbound probes, primers, etc. In some embodiments, the wash is a stringency wash.

Upon addition of a DNA polymerase in the presence of appropriate dNTP precursors and other cofactors, the amplification primer or nucleic acid barcode molecule is elongated by replication of multiple copies of the template (e.g., a concatemer of the template is generated).

In any one of the embodiments herein, the products are generated using rolling circle amplification (RCA). In any one of the embodiments herein, the RCA can comprise a linear RCA, a branched RCA, a dendritic RCA, or any combination thereof. In any one of the embodiments herein, the product can be generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof.

In some embodiments, upon addition of a DNA polymerase in the presence of appropriate dNTP precursors and other cofactors, primers are elongated to produce multiple copies of the circular template. This amplification step can utilize isothermal amplification or non-isothermal amplification. Techniques for rolling circle amplification (RCA) include but are not limited to linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49 (11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29: el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification, modified nucleotides are added to the reaction to incorporate the modified nucleotides in the amplification product (e.g., nanoball). In some instances, the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some embodiments, a plurality of amplification products (e.g., RCA products) are immobilized within the matrix generally at the location of the nucleic acid being amplified, thereby creating a localized colony of amplicons. In some embodiments, the amplification products are immobilized within the matrix by steric factors. In some embodiments, the amplification products are immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress. In some aspects, the amplification products are copolymerized and/or covalently attached to the surrounding matrix.

In some embodiments, rolling circle amplification (RCA) products are generated using the circularized template associated with a nucleic acid barcode molecule or a derivative thereof. In some embodiments, rolling circle amplification (RCA) products are generated using the circularized template bound to a nucleic acid barcode molecule or a derivative thereof. In some embodiments, rolling circle amplification (RCA) products are generated using the circularized template bound to an extended nucleic acid barcode molecule or a derivative thereof. In some embodiments, the RCA products (RCPs) are at a location of the substrate where a plurality of polypeptide-oligonucleotide conjugates are spread. In some embodiments, a sequence of the RCP is analyzed at a location of the substrate. In some embodiments, the sequence of the RCP is analyzed by sequencing by ligation, sequencing by synthesis, sequencing by binding, sequencing by avidity or a combination thereof.

In some embodiments, the analysis and/or sequence determination comprises detecting all or a portion of the generated RCP(s). In some embodiments, the detection or determination comprises imaging the RCP. In some embodiments, the detection or determination is performed by sequencing the nucleic acid barcode molecule or a derivative thereof at the same location as the polypeptide sequence is detected (e.g., as described in Section III.A).

In some embodiments, sequencing comprises incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (i.e., nucleotide sequence) of the incorporated nucleotides or labeled primer extension products can be determined, and consequently, the nucleotide sequence of the corresponding template nucleic acid. Aspects of sequencing are described, for example, in Mitra et al., (2003) Anal. Biochem. 320, 55-65, and Lee et al., (2014) Science, 343 (6177), 1360-1363. In addition, examples of methods and systems for performing sequencing are described in US 2016/0024555, US 2019/0194709, and in U.S. Pat. Nos. 10,138,509, 10,494,662 and 10,179,932, all of which are herein incorporated by reference in their entireties.

In some embodiments, sequencing comprises a base-by-base sequencing method, e.g., sequencing-by-synthesis (SBS), sequencing-by-avidity (SBA) or sequencing-by-binding (SBB). In some embodiments, a sequencing primer is used and base-by-base sequencing using a cyclic series of nucleotide incorporation or binding, respectively, thereby generating extension products of the sequencing primer is performed followed by removing, cleaving, or blocking the extension products of the sequencing primer.

Generally in sequencing-by-synthesis methods, a first population of detectably labeled nucleotides (e.g., dNTPs) are introduced to contact a template nucleotide (e.g., a barcode sequence in the RCP) hybridized to a sequencing primer, and a first detectably labeled nucleotide (e.g., A, T, C, or G nucleotide) is incorporated by a polymerase to extend the sequencing primer in the 5′ to 3′ direction using a complementary nucleotide (a first nucleotide residue) in the template nucleotide as template. A signal from the first detectably labeled nucleotide can then be detected. The first population of nucleotides may be continuously introduced, but in order for a second detectably labeled nucleotide to incorporate into the extended sequencing primer, nucleotides in the first population of nucleotides that have not incorporated into a sequencing primer are generally removed (e.g., by washing), and a second population of detectably labeled nucleotides are introduced into the reaction. Then, a second detectably labeled nucleotide (e.g., A, T, C, or G nucleotide) is incorporated by the same or a different polymerase to extend the already extended sequencing primer in the 5′ to 3′ direction using a complementary nucleotide (a second nucleotide residue) in the template nucleotide as template. Thus, in some embodiments, cycles of introducing and removing detectably labeled nucleotides are performed.

In some embodiments, the detectably labeled nucleotide comprises a fluorescent dye. Commercially available fluorescent dyes include, but are not limited to 4,7-dichlorofluorescein dyes, spectrally resolvable rhodamine dyes, 4,7-dichlororhodamine dyes, cyanine dyes, ether-substituted fluorescein dyes, energy transfer dyes, and xanthine dyes. Labeling can also be carried out with quantum dots. In some embodiments, a fluorescent label comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

In some embodiments, the detectably labeled nucleotide is a fluorescent nucleotide analog. Without being bound by theory, a fluorescent nucleotide analog is readily incorporated into a nucleotide, polynucleotide, and/or oligonucleotide sequence during extension. Examples of commercially available fluorescent nucleotide analogues readily incorporated into nucleotide and/or polynucleotide sequences comprise, but are not limited to, Cy3TM-dNTP (cyanine 3-dNTP), Cy3TM-dNTP (cyanine 3-dNTP), Cy5TM-dNTP (cyanine 5-dNTP), Cy5TM-dNTP (cyanine 5 dNTP) (Amersham Biosciences, Piscataway, N.J.), fluorescein-12-dNTP, tetramethylrhodamine-6-dNTP, TEXAS RED®-5-dNTP (red fluorescent dye-dNTP), CASCADE® BLUE-7-dNTP (blue fluorescent dye-dNTP), BODIPY™ FL-14-dNTP (green fluorescent dye-dNTP), BODIPY™ TMR-14-dNTP (orange fluorescent dye-dNTP), BODIPY™ TR-14-dNTP (red fluorescent dye-dNTP), RHODAMINE GREEN™-5-dNTP (green fluorescent dye-dNTP), OREGON GREEN™ 488-5-dNTP (green fluorescent dye-dNTP), TEXAS RED™-12-dNTP (red fluorescent dye-dNTP), BODIPY™ 630/650-14-dNTP (far red fluorescent dye-dNTP), BODIPY™ 650/665-14-dNTP (far red fluorescent dye-dNTP), ALEXA FLUOR™ 488-5-dNTP (green fluorescent dye-dNTP), ALEXA FLUOR™ 532-5-dNTP (yellow fluorescent dye-dNTP), ALEXA FLUOR™ 568-5-dNTP (red/orange fluorescent dye-dNTP), ALEXA FLUOR™ 594-5-dNTP (red fluorescent dye-dNTP), ALEXA FLUOR™ 546-14-dNTP (orange fluorescent dye-dNTP), fluorescein-12-UTP, tetramethylrhodamine-6-UTP, TEXAS RED™-5-UTP (red fluorescent dye-UTP), mCherry, CASCADE® BLUE-7-UTP (blue fluorescent dye-UTP), BODIPY™ FL-14-UTP (green fluorescent protein-UTP), BODIPY™ TMR-14-UTP (orange fluorescent dye-UTP), BODIPY™ TR-14-UTP (red fluorescent dye-UTP), RHODAMINE GREEN™-5-UTP (green fluorescent dye-UTP), ALEXA FLUOR™ 488-5-UTP (green fluorescent dye-UTP), and ALEXA FLUOR™ 546-14-UTP (orange fluorescent dye-UTP) (Molecular Probes, Inc. Eugene, Oreg.). Methods are known for custom synthesis of nucleotides having other fluorophores.

In some embodiments, the base-by-base sequencing comprises using a polymerase that is fluorescently labeled. In some embodiments, the base-by-base sequencing comprises using a polymerase-nucleotide conjugate comprising a fluorescently labeled polymerase linked to a nucleotide moiety that is not fluorescently labeled. In some embodiments, the base-by-base sequencing comprises using a multivalent polymer-nucleotide conjugate comprising a polymer core, multiple nucleotide moieties, and one or more fluorescent labels.

In some embodiments, sequencing is performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Examples of SBS methods comprise those described for example, but not limited to, US 2007/0166705, US 2006/0188901, U.S. Pat. No. 7,057,026, US 2006/0240439, US 2006/0281109, US 2011/005986, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequencing is performed by sequencing-by-binding (SBB). Various aspects of SBB are described in U.S. Pat. No. 10,655,176 B2, the content of which is herein incorporated by reference in its entirety. In some embodiments, SBB comprises performing repetitive cycles of detecting a stabilized complex that forms at each position along the template nucleic acid to be sequenced (e.g., a ternary complex that includes the primed template nucleic acid, a polymerase, and a cognate nucleotide for the position), under conditions that prevent covalent incorporation of the cognate nucleotide into the primer, and then extending the primer to allow detection of the next position along the template nucleic acid. In the sequencing-by-binding approach, detection of the nucleotide at each position of the template occurs prior to extension of the primer to the next position. Generally, the methodology is used to distinguish the four different nucleotide types that can be present at positions along a nucleic acid template by uniquely labelling each type of ternary complex (i.e. different types of ternary complexes differing in the type of nucleotide it contains) or by separately delivering the reagents needed to form each type of ternary complex. In some instances, the labeling may comprise fluorescence labeling of, e.g., the cognate nucleotide or the polymerase that participate in the ternary complex.

In some embodiments, sequencing is performed by sequencing-by-avidity (SBA). Some aspects of SBA approaches are described in U.S. Pat. No. 10,768,173 B2, the content of which is herein incorporated by reference in its entirety. In some embodiments, SBA comprises detecting a multivalent binding complex formed between a fluorescently-labeled polymer-nucleotide conjugate, and a one or more primed nucleic acid sequences. Fluorescence imaging is used to detect the bound complex. Following the imaging step, the multivalent binding complex is disrupted and washed away, the correct blocked nucleotide is incorporated into the primer extension strand, and the sequencing cycle is repeated.

In some embodiments, sequencing is performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309:1728-1732, and in U.S. Pat. Nos. 5,599,675; 5,750,341; 6,969,488; 6,172,218; and 6,306,597.

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181.

In some embodiments, the detecting comprises a plurality of repeated cycles of hybridization and removal of probes (e.g., detectably labeled probes, or intermediate probes that bind to detectably labeled probes) to the RCA product generated using the circularizable probe hybridized to the nucleic acid barcode molecule.

Methods for binding a sequence that uses various probes or oligonucleotides have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety. Detectably-labeled probes can be useful for detecting multiple sequences and be detected in one or more hybridization cycles (e.g., sequential hybridization assays, or sequencing by hybridization).

In some embodiments, the detecting comprises binding an intermediate probe directly or indirectly to the RCA product generated using the circularizable probe hybridized to the nucleic acid barcode molecule, binding a detectably labeled probe directly or indirectly to a detection region of the intermediate probe, and detecting a signal associated with the detectably labeled probe. n some embodiments, the method comprises performing one or more wash steps to remove unbound and/or nonspecifically bound intermediate probe molecules from the RCA product generated using the circularizable probe hybridized to the nucleic acid barcode molecule.

In some embodiments, the detecting comprises: detecting signals associated with detectably labeled probes that are hybridized to the RCA product generated using the circularizable probe hybridized to the nucleic acid barcode molecule and/or detecting signals associated with detectably labeled probes that are hybridized to intermediate probes which are in turn hybridized to the RCA product generated using the circularizable probe hybridized to the nucleic acid barcode molecule. In some embodiments, the detectably labeled probes is fluorescently labeled.

In some embodiments, the methods comprise detecting the sequence in all or a portion of the RCA product generated using the circularizable probe hybridized to the nucleic acid barcode molecule. In some embodiments, the detection step involves sequencing by hybridization, sequencing by ligation, sequencing by synthesis, sequencing by binding, and/or hybridization-based sequence detection. In some embodiments, the detection comprises sequential fluorescent hybridization of probes (e.g., for combinatorial decoding of the barcode sequence or complement thereof).

In some embodiments, the detection or determination the sequence of the nucleic acid barcode molecule comprises hybridizing to the RCA product generated using the circularizable probe hybridized to the nucleic acid barcode molecule directly or indirectly a detection oligonucleotide labeled with a fluorophore, an isotope, a mass tag, or a combination thereof. In some embodiments, the detection or determination comprises imaging.

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, and/or light-field microscopy.

In some embodiments, fluorescence microscopy is used for detection and imaging. Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM).

### IV. Samples

In some embodiments, the single cell is from a biological sample. In some instances, a biological sample is dissociated into a plurality of cells comprising the single cell. In some instances, the plurality of cells are dissociated from the biological sample into single cells using one or more microfluidic channels. In some instances, the biological sample is a cell culture sample. In some instances, the biological sample is a fresh tissue sample and/or a frozen tissue sample, wherein the biological sample is a cell suspension, culture of cells, tissue sample, or a bodily fluid. In some embodiments, the digested polypeptide is from a cell surface protein. In some embodiments, the digested polypeptide is from an intracellular protein. In some embodiments, the digested polypeptide is from an extracellular protein.

A sample disclosed herein can be or derived from any biological sample. In some instances, a biological sample is obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. In some embodiments, the biological sample is obtained as a tissue sample, such as a tissue section, biopsy, a core biopsy, needle aspirate, or fine needle aspirate. In some embodiments, the biological sample is or comprise a cell pellet or a section of a cell pellet. In some embodiments, the biological sample is or comprise a cell block or a section of a cell block. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some instances, the sample is a plasma sample comprising a plurality of polypeptides. In some instances, the sample has been processed, e.g., to remove overly abundant proteins (e.g., using standard immunoaffinity methods). In some cases, depletion of abundant proteins can be useful for plasma samples (e.g., to remove albumin and immunoglobulins).

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

A sample may be a fixed sample. For example, a sample may comprise a plurality of fixed samples, such as a plurality of fixed cells or fixed nuclei. Alternatively, or in addition, a sample may comprise a fixed tissue. Fixation of cell or cellular constituent, or a tissue comprising a plurality of cells or nuclei, may comprise application of a chemical species or chemical stimulus. The term “fixed” as used herein with regard to biological samples generally refers to the state of being preserved from decay and/or degradation. “Fixation” generally refers to a process that results in a fixed sample, and in some instances can include contacting the biomolecules within a biological sample with a fixative (or fixation reagent) for some amount of time, whereby the fixative results in covalent bonding interactions such as crosslinks between biomolecules in the sample. A “fixed biological sample” may generally refer to a biological sample that has been contacted with a fixation reagent or fixative. For example, a formaldehyde-fixed biological sample has been contacted with the fixation reagent formaldehyde. “Fixed cells” or “fixed tissues” generally refer to cells or tissues that have been in contact with a fixative under conditions sufficient to allow or result in the formation of intra- and inter-molecular covalent crosslinks between biomolecules in the biological sample. Generally, contact of biological sample (e.g., a cell or nucleus) with a fixation reagent (e.g., paraformaldehyde or PFA) results in the formation of intra- and inter-molecular covalent crosslinks between biomolecules in the biological sample. In some cases, provision of the fixation reagent, such as formaldehyde, may result in covalent aminal crosslinks within RNA, DNA, and/or protein molecules. For example, the widely used fixative reagent, paraformaldehyde or PFA, fixes tissue samples by catalyzing crosslink formation between basic amino acids in proteins, such as lysine and glutamine. Both intra-molecular and inter-molecular crosslinks can form in the protein. These crosslinks can preserve protein secondary structure and also eliminate enzymatic activity in the preserved tissue sample. Examples of fixation reagents include but are not limited to aldehyde fixatives (e.g., formaldehyde, also commonly referred to as “paraformaldehyde,” “PFA,” and “formalin”; glutaraldehyde; etc.), imidoesters, NHS (N-Hydroxysuccinimide) esters, and the like.

Other examples of fixation reagents include, for example, organic solvents such as alcohols (e.g., methanol or ethanol), ketones (e.g., acetone), and aldehydes (e.g., paraformaldehyde, formaldehyde (e.g., formalin), or glutaraldehyde). As described herein, cross-linking agents may also be used for fixation including, without limitation, disuccinimidyl suberate (DSS), dimethylsuberimidate (DMS), formalin, and dimethyladipimidate (DMA), dithio-bis (-succinimidyl propionate) (DSP), disuccinimidyl tartrate (DST), and ethylene glycol bis(succinimidyl succinate) (EGS). In some cases, a cross-linking agent may be a cleavable cross-linking agent (e.g., thermally cleavable, photocleavable, etc.). In some cases, more than one fixation reagent can be used in combination when preparing a fixed biological sample. Changes to a characteristic or a set of characteristics of a cell or cellular constituents (e.g., incurred upon interaction with one or more fixation agents) may be at least partially reversible (e.g., via rehydration or de-crosslinking). Alternatively, changes to a characteristic or set of characteristics of a cell or cellular constituents (e.g., incurred upon interaction with one or more fixation agents) may be substantially irreversible.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

To facilitate visualization, biological samples can be stained using a wide variety of stains and staining techniques. In some embodiments, for example, a sample can be stained using any number of stains and/or immunohistochemical reagents. In some embodiments, the sample is contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, the stain is performed using a lipophilic dye. In some examples, the staining is performed with a lipophilic carbocyanine or aminostyryl dye, or analogs thereof (e.g, DiI, DIO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples is destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65 (8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

In some embodiments, the digested polypeptides are derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

### V. Compositions, Systems and Kits

Provided herein are compositions, systems and kits for generating a polypeptide-oligonucleotide conjugate by coupling a digested polypeptide from a cell to a nucleic acid barcode molecule using a partition. In some embodiments, the compositions, systems and kits comprising reagents for forming a droplet population are provided. In some embodiments, the compositions, systems and kits comprises a plurality of wells. For example, the compositions, systems and kits comprise instructions for use, a microwell array or device, and reagents (e.g., beads). In some embodiments, the compositions, systems and kits comprise any useful reagents for performing the processes described herein, e.g., nucleic acid reactions, barcoding digested polypeptides with nucleic acid barcode molecules, sample processing (e.g., for cell lysis, fixation, and/or permeabilization). In some embodiments, the compositions, systems and kits comprises a conjugation reagent as described in Section II. In some embodiments, the compositions, systems and kits comprise a reducing agent as described in Section II. In some embodiments, the compositions, systems and kits comprise a single particle (e.g., bead) coupled to a first nucleic acid barcode molecule for analysis of a first analyte species (e.g., polypeptide) and a second nucleic acid barcode molecule for analysis of a second analyte species (e.g., nucleic acid). In some embodiments, the compositions, systems and kits comprise a plurality of particles as described in Section II.F.

Provided herein are compositions, systems and kits for analyzing a polypeptide-oligonucleotide conjugate according to any of the methods described herein. Provided herein are compositions, systems and kits for analyzing a polypeptide-oligonucleotide conjugate according to any of the methods described herein and for analyzing a sequence of the first barcoded nucleic acid molecule and of the second barcoded nucleic acid molecule generated as described in Section II.F. In some embodiments, provided herein are compositions, systems or kits, for example comprising a plurality of binding agents for detecting the digested polypeptides or portions thereof. In some embodiments, provided herein are compositions, systems or kits, for example comprising a plurality of circularizable probes as described in Section III. In some embodiments, the compositions, systems or kits comprise a ligase and/or a polymerase. In some embodiments, the compositions, systems or kits comprise a plurality of dNTPs. In some embodiments, disclosed herein is a system that comprises a plurality of amplification products each containing monomeric units of a sequence associated with a cell barcode sequence of a nucleic acid barcode molecule. In some embodiments, the plurality of amplification products is formed using the plurality of circularizable probes and any one of the amplification techniques described herein.

Provided herein are compositions, systems and kits, for example comprising the plurality of circularizable probes, e.g., any described in Section III, and reagents for performing the methods provided herein, for example reagents required for one or more steps comprising hybridization, ligation, gap fill, amplification, detection, sequencing, and/or sample preparation as described herein. In some embodiments, the system or kit comprises a plurality of nucleic acid barcode molecules coupled to a particle. In some embodiments, the system or kit comprises a ligase for forming a circular template for RCA and/or a polymerase for performing RCA.

Provided herein are compositions, systems and kits, for processing or sequencing a polypeptide or for generating the modified amino acids or derivatives thereof. For example, provided are reagents for generating a polymerizable molecule comprising one or more modified amino acids. In some instances, the compositions, systems and kits comprise a linker (e.g., to couple to an amino acid), which linker may comprise a polymerizable molecule, a cleaving agent, a ligating agent (e.g., ligase), a capture moiety, reagents for conducting a reaction, or a combination thereof. In some instances, the compositions, systems and kits comprise a linker comprising (e.g., covalently attached thereto) a nucleic acid barcode molecule, wherein the linker comprises an amino acid-reactive group.

In some embodiments, the compositions, systems and kits comprise reagents and/or consumables required for performing one or more steps of the provided methods. In some embodiments, the compositions, systems and kits comprise reagents for fixing, embedding, and/or permeabilizing cells. In some embodiments, the compositions, systems and kits comprise reagents, such as enzymes and buffers for ligation and/or amplification, such as ligases and/or polymerases. In some aspects, the compositions, systems and kits comprise any of the reagents described herein, e.g., wash buffer and ligation buffer. In some embodiments, the compositions, systems and kits comprise reagents for detection and/or sequencing (e.g., as described in Section III).

### VI. Systems and Instruments

The present disclosure provides systems and/or instruments for performing methods of the disclosure. In some embodiments, systems and/or instruments are configured to form and/or process a droplet population (e.g., as described in Section II). In some embodiments, systems and/or instruments are configured to control a microfluidics system (e.g., fluid flow). In some embodiments, systems and/or instruments are configured to polymerize droplets. In some embodiments, systems and/or instruments are configured to perform sequencing applications In some embodiments, systems and/or instruments are configured to deliver reagents to a droplet population. In some embodiments, systems and/or instruments are configured to regulate fluid flow rate in one or more channels in a microfluidic structure.

Provided herein is an instrument having integrated optics and fluidics modules (an “opto-fluidic instrument” or “opto-fluidic system”) for detecting polypeptide-oligonucleotide conjugates as described herein. In an opto-fluidic instrument, the fluidics module is configured to deliver one or more reagents (e.g., detectably labeled probes) to the sample and/or remove spent reagents therefrom. Additionally, the optics module is configured to illuminate the sample with light having one or more spectral emission curves (over a range of wavelengths) and subsequently capture one or more images of emitted light signals (e.g., signals for detection described in Section III). In various embodiments, the captured images may be processed in real time and/or at a later time to determine the presence of the one or more target molecules in the biological sample, as well as three-dimensional position information associated with each detected target molecule. Additionally, the opto-fluidics instrument includes a sample module configured to receive (and, optionally, secure) one or more substrates. In some instances, the sample module includes an X-Y stage configured to move the substrate along an X-Y plane (e.g., perpendicular to an objective lens of the optics module).

In some embodiments, systems and/or instruments are configured for detection of amplification products as described in Section III. In some embodiments, systems and/or instruments comprise a fluidics module that includes fluids needed for establishing the experimental conditions required for the probing of digested polypeptides and/or determining the sequence of a nucleic acid barcode molecule. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

In various embodiments, the opto-fluidic instrument is configured to facilitate the experimental conditions conducive for the processing and/or detection of a plurality of polypeptide-oligonucleotide conjugates. For example, the opto-fluidic instrument can include a fluidics module, an optics module, a sample module, and an ancillary module, and these modules may be operated by a system controller to create the experimental conditions. In various embodiments, the various modules of the opto-fluidic instrument may be separate components in communication with each other, or at least some of them may be integrated together.

In various embodiments, the sample module may be configured to receive the sample into the opto-fluidic instrument. For instance, the sample module may include a sample interface module (SIM) that is configured to receive a sample device (e.g., cassette) onto which the sample can be deposited. That is, the sample may be placed in the opto-fluidic instrument by depositing the sample (e.g., comprising polypeptide-oligonucleotide conjugates) on a sample device that is then inserted into the SIM of the sample module. In some instances, the sample module may also include an X-Y stage onto which the SIM is mounted. The X-Y stage may be configured to move the SIM mounted thereon (e.g., and as such the sample device containing the sample inserted therein) in perpendicular directions along the two-dimensional (2D) plane of the opto-fluidic instrument.

The experimental conditions that are conducive for the detection of polypeptide-oligonucleotide conjugates on the substrate may depend on the technique that is employed by the opto-fluidic instrument. For example, in various embodiments, the opto-fluidic instrument can be a system that is configured to detect amplification products in the sample via hybridization of probes. In some embodiments, the opto-fluidic instrument is configured to detect a sequence of the first barcoded nucleic acid molecule and the barcoded nucleic acid molecule generated as described in Section II.F. In such cases, the experimental conditions can include molecule hybridization conditions that result in the intensity of hybridization of the amplification product to a probe (e.g., detectably labeled probe) being significantly higher when the detectably labeled probe sequence is complementary to the amplification product than when there is a single-base mismatch. The hybridization conditions include the preparation of the sample using reagents such as washing/stripping reagents, hybridizing reagents, extension reagents etc., and such reagents may be provided by the fluidics module.

In some embodiments, systems and/or instruments are configured to store the reagents, as well as for transporting said reagents to and from the sample. For example, the fluidics module may include reservoirs configured to store the reagents, as well as a waste container configured for collecting the reagents (e.g., and other waste). Further, the fluidics module may also include pumps, tubes, pipettes, etc., that are configured to facilitate the transport of the reagent to the sample device (e.g., and as such the sample). For instance, the fluidics module may include pumps (“reagent pumps”) that are configured to pump washing/stripping reagents to the sample device for use in washing/stripping the sample (e.g., as well as other washing functions such as washing an objective lens of the imaging system of the optics module).

In various embodiments, the ancillary module can be a cooling system of the opto-fluidic instrument, and the cooling system may include a network of coolant-carrying tubes that are configured to transport coolants to various modules of the opto-fluidic instrument for regulating the temperatures thereof. In such cases, the fluidics module may include coolant reservoirs for storing the coolants and pumps (e.g., “coolant pumps”) for generating a pressure differential, thereby forcing the coolants to flow from the reservoirs to the various modules of the opto-fluidic instrument via the coolant-carrying tubes. In some instances, the fluidics module may include returning coolant reservoirs that may be configured to receive and store returning coolants, e.g., heated coolants flowing back into the returning coolant reservoirs after absorbing heat discharged by the various modules of the opto-fluidic instrument. In such cases, the fluidics module may also include cooling fans that are configured to force air (e.g., cool and/or ambient air) into the returning coolant reservoirs to cool the heated coolants stored therein. In some instances, the fluidics module may also include cooling fans that are configured to force air directly into a component of the opto-fluidic instrument so as to cool said component. For example, the fluidics module may include cooling fans that are configured to direct cool or ambient air into the system controller to cool the same.

As discussed above, the opto-fluidic instrument may include an optics module which include the various optical components of the opto-fluidic instrument, such as but not limited to a camera, an illumination module (e.g., LEDs), an objective lens, and/or the like. The optics module may include a fluorescence imaging system. In some instances, the optics module may also include an optical frame onto which the camera, the illumination module, and/or the X-Y stage of the sample module may be mounted.

In various embodiments, the system controller may be configured to control the operations of the instrument (e.g., and the operations of one or more modules thereof). In some instances, the system controller may take various forms, including a processor, a single computer (or computer system), or multiple computers in communication with each other. In various embodiments, the system controller may be communicatively coupled with data storage, set of input devices, display system, or a combination thereof. In some cases, some or all of these components may be considered to be part of or otherwise integrated with the system controller, may be separate components in communication with each other, or may be integrated together. In other examples, the system controller can be, or may be in communication with, a cloud computing platform.

In various embodiments, the opto-fluidic instrument may analyze the sample and may generate the output associated with polypeptide-oligonucleotide conjugates. In some instances, the output may include optical signatures associated with and/or for identification of a polypeptide (e.g., peptide or protein). In some instances, the output may include optical signatures associated with a nucleic acid barcode molecule and/or for identification of the origin of polypeptide (e.g., peptide or protein). In some instances, the output may include optical signatures associated with and/or trace the origin of the polypeptide (e.g., peptide or protein).

### VII. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” as used herein, in some embodiments, is an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

In some instances, “ligation” refers to the formation of a covalent bond or linkage between the termini of two or more nucleic acids, e.g., oligonucleotides and/or polynucleotides, in a template-driven reaction. The nature of the bond or linkage may vary widely and the ligation, in some embodiments, is carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

“A and/or B” is used herein to include all of the following alternatives: “A”, “B”, “A or B”, and “A and B”

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value or rounded to the nearest significant figure, in all cases inclusive of the provided value.

Headings, e.g., (a), (b), (i) etc., are presented merely for ease of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include polynucleotide barcodes, random nucleic acid and/or amino acid sequences, and synthetic nucleic acid and/or amino acid sequences. A barcode can be attached to an analyte in a reversible or irreversible manner. Barcodes can allow for identification and/or quantification of individual sequencing-reads.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively, or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “biological particle” may be used herein to generally refer to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a nucleus of a cell. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell but may not include other constituents of the cell. An example of such constituents is a nucleus or an organelle. A cell may be a live cell. The live cell may be capable of being cultured, for example, being cultured when enclosed in a gel or polymer matrix or cultured when comprising a gel or polymer matrix.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition can be a physical container, compartment, or vessel, such as a droplet, a flowcell, a reaction chamber, a reaction compartment, a tube, a well, or a microwell. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

### EXAMPLES

The following examples are included for illustrative purposes only and are not intended to limit the scope of the present disclosure.

### Example 1: Sample Preparation in Partition for Polypeptide Analysis

This Example describes a workflow for preparing a sample for analysis of proteins from single cells. Digested polypeptides from a single cell are barcoded using a plurality of nucleic acid barcode molecules comprising the same cell barcode sequence. The workflow for barcoding proteins is performed in partitions (e.g., in droplets or wells) and the generated barcoded polypeptides are used for downstream protein sequencing.

Nucleic acid barcode molecules are conjugated by a disulfide bond to a substrate as shown in FIG. 1A, such as a gel bead. The nucleic acid barcode molecules coupled to the substrate (e.g., a particle such as a gel bead) are provided in partitions whereby the partition also includes a single cell. In some cases, rather than the intact cell, the partition includes biological particles encapsulated within a particulate material to form a “cell bead.” The partition may be a well or a droplet. In the partition, appropriate conditions are applied to lyse the single cell and release cellular components into the partition (e.g., well or droplet). The cells in droplets with the particle are also treated with trypsin. In the partition while cells are lysed and treated with trypsin, the substrate (e.g., gel bead) remains intact as proteins are digested to generate a plurality of digested polypeptides. The nucleic acid barcode molecules coupled to the particle comprises: Gel bead—5′ acrydite—disulfide linkage—internal alkyne—cell barcode (CBC) and functional sequence(s).

After the proteins are treated with trypsin, a diazo transfer reagent is added and then quenched (e.g., DBCO-constrained alkyne). In examples using wells as partitions, the conjugation reagent and reducing agent are added directly to the contents of the well. For treatment of cell beads in the partition, the conjugation reagent and reducing agent are added to the partition and multistep reactions (including washing and buffer exchange) are carried out while keeping contents of cells intact in a porous partition (e.g., the cell bead). In some aspects, cell beads eliminate the need to quench reactive reagents and the process of digesting proteins to polypeptides, conjugating C-terminus nucleic acid barcode molecules and any intermediate washes are performed in a stepwise fashion.

For using droplets as partitions, two micellar additions are used to transfer reagents to the initial droplets comprising contents from the single cell. To introduce the conjugation reagent and/or the reducing agent, a water-in-fluorinated oil emulsion is created by agitation (e.g., shaking) to produce droplets which contained a disulfide bond reducing agent (dithiothreitol; DTT) or the conjugation reagent (reagents for carbodiimide conjugation e.g., EDC, DCC). In certain embodiments, the reducing agent is selected from the group consisting of 2-mercaptoethanol (BME), tris (2-carboxyethyl) phosphine hydrochloride (TCEP), and dithiothreitol (DTT), or a functional derivative thereof. In certain embodiments, the conjugation reagent is selected from the group consisting of 1-Ethyl-3-(3-dimethylaminopropyl) carbodiimide (EDC), dicyclohexylcarbodiimide (DCC), and diisopropylcarbodiimide (DIC). The droplet population comprising cell contents is then combined with the droplets comprising the DTT and mixed together in the same tube. This provides a way to transfer DTT from one droplet population to another and subsequently dissolve the beads through reduction of their crosslinking disulfide bonds, making the nucleic acid barcode molecules available for conjugation to digested polypeptides from the single cell while maintaining the proteins from one cell and nucleic acid barcode molecules that share a common cell barcode in the same partition. A first micellar transfer provides a conjugation reagent to the partition, e.g., addition of diazo transfer reagent, to react with the digested polypeptides. In some cases, covalent lysine chemistry is used to add a bio-orthogonal reactive group (e.g., click chemistry reagent) to C-terminal lysines revealed by trypsin treatment in the digested polypeptides. The conjugation reagent is provided to the partition in the absence of an activating reagent (e.g., copper). A second micellar transfer provides copper and a reducing agent (e.g., DTT) to release the nucleic acid barcode molecules from the substrate (e.g., dissolve gel beads, thereby releasing the nucleic acid barcode molecules).

Using any of the reagent transfer workflows, the released nucleic acid barcode molecules are coupled to the digested polypeptides with the added reactive group. In some cases, the COOH at the terminus of the digested polypeptide is coupled to the nucleic acid barcode molecule. as opposed to the amino lysine. After the transfer, a plurality of digested polypeptides from a single cell are labeled with nucleic acid barcode molecules having the sequence associated with the originating single cell. Once the generated polypeptide-oligonucleotide conjugates are formed, the partition is removed (e.g., by breaking the emulsion or pooling contents of different partitions (e.g., pooling wells)).

The nucleic acid barcode molecule coupled to the digested polypeptide in the polypeptide-oligonucleotide conjugate is then coupled to a substrate (e.g., via a thiol group at the 5′ end of the nucleic acid barcode molecule) for downstream analysis. For example, the nucleic acid barcode molecule of the polypeptide-oligonucleotide conjugate has a free 3′ end for extension (e.g., by ligation or by polymerase).

### Example 2: Polypeptide Sequence Detection

This Example describes analysis of the generated barcoded polypeptides (e.g., polypeptide-oligonucleotide conjugate) for protein sequencing.

The sequence of amino acids in the digested polypeptide of the polypeptide-oligonucleotide conjugate generated as described in Example 1 is determined using binding agents that recognizes at least one of the amino acids of the polypeptides (e.g., binds to a terminal amino acid of the digested polypeptide or a derivative thereof). The conjugated nucleic acid barcode molecule conjugated to the polypeptide has a free 3′ end for extension (e.g., by ligation or by polymerase). A binding agent comprises a coding tag sequence associated with the binding agent (e.g., corresponding to the moiety bound by the binding agent). The coding tag sequence of the binding agent is transferred by ligation or polymerase extension to the nucleic acid barcode molecule to generate an extended nucleic acid barcode molecule. Multiple cycles of the binding agent contacting and transfer of the coding tag sequence is performed and a terminal amino acid or terminal amino acid derivative is cleaved from the digested polypeptide at the end of each cycle. For example, polypeptide sequence determination in a cyclic manner is described in U.S. Patent Application Publication No. 2024/0377409 and 2025/0051756, which are entirely incorporated herein by reference for all purposes. For example, N-terminal Edman degradation is compatible with nucleic acid barcoding and is used for cycles of polypeptide sequencing. The nucleic acid barcode molecule conjugated to the digested polypeptides and any extensions thereof encoding the amino acids of the polypeptide are sequenced. The sequence associated with the polypeptide or portion thereof allows the common cell barcode in the nucleic acid barcode molecule to associate a plurality of polypeptides that originated in the same partition.

The sequence of amino acids in the digested polypeptide of the polypeptide-oligonucleotide conjugate generated as described in Example 1 alternatively is determined using a nanopore, e.g., as described in U.S. Patent Application Publication No. 2024/0337660, which is hereby incorporated by reference in its entirety. The sequence of amino acids in the digested polypeptide of the polypeptide-oligonucleotide conjugate generated as described in Example 1 is alternatively used in a workflow for detecting a cleaved terminal amino acid or terminal amino acid derivative from the digested polypeptide, e.g., as described in U.S. Patent Application Publication No. 2024/0409995, which is hereby incorporated by reference in its entirety.

### Example 3: Imaging-Based Polypeptide Sequence Detection

This Example describes analysis of the generated barcoded polypeptides (e.g., polypeptide-oligonucleotide conjugate) using an imaging-based readout. A plurality of digested polypeptides from a single cell are in a partition, wherein the partition comprises a plurality of nucleic acid barcode molecules unique to the partition and the contents of the partition are contacted with a reducing agent and a conjugation reagent to generate a plurality of polypeptide-oligonucleotide conjugates by coupling the plurality of digested polypeptides to the plurality of nucleic acid barcode molecules. The polypeptide-oligonucleotide conjugates generated are pooled from a plurality of partitions (e.g., droplets or wells) and dispersed on a substrate (e.g., a glass surface such as a slide). A plurality of signals associated with (i) the digested polypeptide (e.g., particular amino acid(s)), and (ii) the nucleic acid barcode molecule are detected at a location on the substrate by imaging the conjugates spread on the substrate.

The digested polypeptide sequence or portion thereof is determined using any suitable detection methods, such as binding agents that recognize at least one of the amino acids of the polypeptides (e.g., binds to a terminal amino acid of the digested polypeptide or a derivative thereof). For example, detecting the sequence of the digested polypeptide comprises fluorosequencing. e.g., as described in U.S. Patent Application Publication No. 2024/0201198, which is hereby incorporated by reference in its entirety. In some cases, the terminal amino acid of the digested polypeptides on the substrate or a derivative thereof are contacted with a binding agent. The binding agent is detectably labeled (e.g., with a fluorophore) and the terminal amino acid or terminal amino acid derivative of the digested polypeptide is detected either while in the conjugate or after it is cleaved (e.g., as described in Example 2) from the digested polypeptide.

The nucleic acid barcode molecule of the generated conjugate is detected using a circularizable probe (e.g., a padlock probe) to amplify the sequence of the nucleic acid barcode molecule. A rolling circle amplification (RCA) is performed by binding a circularizable probe to the nucleic acid barcode molecule, wherein the circularizable probe comprises a 5′ hybridization region and a 3′ hybridization region that binds a first common sequence and a second common sequence, respectively, in the nucleic acid barcode molecule, as shown in FIG. 5. Upon hybridization, the end of the circularizable probe is gap filled using the nucleic acid barcode molecule as template. Using the common sequences and gap fill approach, different nucleic acid barcode molecules comprising different cell barcode (CBC) sequences corresponding to different single cells (and corresponding partitions) can utilize the same circularizable probe with common sequences as binding arms. The extended circularizable probe with the CBC incorporated is then ligated to generate a circularized probe. The circularized probe serves as a template for RCA and RCA is performed by extending the 3′ end of the nucleic acid barcode molecule of the polypeptide-oligonucleotide conjugate on the substrate as a primer. Detecting the plurality of signals associated with the nucleic acid barcode molecule comprises performing sequential hybridization of probes, sequencing by ligation, sequencing by synthesis, sequencing by binding, sequencing by avidity, or a combination thereof. For example, a common sequencing primer is used to bind at least a portion of the generated RCA products on the substrate. Imaging is performed to detect a sequence of the RCA product at the location on the substrate.

Polypeptide detection and nucleic acid barcode (e.g., CBC sequence) detection on the substrate can be performed in either order. In some cases, the signals expected to give higher signal can be performed first to allow identification of locations of the conjugates on the substrate, which can be used to detect subsequent signals (e.g., lower signal to noise modality) associated with the conjugate. In some examples, signals associated with the polypeptide is detected after the nucleic acid barcode sequence is detected.

### Example 4: Sample Preparation for Single-Cell Polypeptide and Nucleic Acid Analysis

This example describes a workflow for parallel barcoding of polypeptides and nucleic acids derived from a single cell for analysis. An outline of the exemplified workflow is illustrated in FIG. 6.

A plurality of cells is fixed and subjected to reverse transcription to generate cDNA within the cells (e.g., from mRNA).

A partition (e.g., droplet) among a plurality of partitions is provided, the partition comprising a single cell of the plurality of cells, and a single particle coupled to both first nucleic acid barcode molecules and second nucleic acid barcode molecules. The first nucleic acid barcode molecules are coupled to the particle via a cleavable moiety comprising a disulfide bond. The second nucleic acid barcode molecules are coupled to the particle without the cleavable moiety. The first and second nucleic acid barcode molecules each include a partition-specific barcode.

The cell is lysed within the partition, thereby releasing proteins and cDNA molecules from the cell. The released cDNA molecules hybridize to the second nucleic acid barcode molecules coupled to the particle. For example, a capture sequence present on the second nucleic acid barcode molecules can hybridize directly or indirectly (e.g., via a splint molecule) to the cDNA. The cDNA molecules remain hybridized to the second nucleic acid barcode molecules coupled to the particle during the protein barcoding steps described below.

The released proteins are digested with trypsin within the partition to generate polypeptides. The trypsin can be co-partitioned with the cell and the particle upon the initial generation of the partition, or can be added subsequently. The first nucleic acid barcode molecules are released from the particle, for example using a reducing agent (e.g., DTT) to reduce the disulfide bond of the cleavable moiety (e.g., break the disulfide bond). In addition, one or more conjugation reagents are added to the partition (e.g., directly for wells, or via micellar delivery for droplets, generally as described in the Examples above) to conjugate the digested polypeptides to the first nucleic acid barcode molecules. The conjugation reagents can be used to facilitate a bio-orthogonal reaction (e.g., using click chemistry) to achieve conjugation of the digested polypeptides to the first nucleic acid barcode molecules to generate polypeptide-oligonucleotide conjugates, generally as described in the Examples above.

The partition is broken and a bead pull-down step is performed to separate the cDNA molecules from the polypeptide-oligonucleotide conjugates for parallel processing in separate reaction vessels. The cDNA molecules are separated from the polypeptide-oligonucleotide conjugates by virtue of the cDNA molecules being coupled to the bead (via hybridization to the second nucleic acid barcode molecules) and the polypeptide-oligonucleotide conjugates being not coupled to the bead. For example, the cDNAs coupled to the bead can be separated from the polypeptide-oligonucleotide conjugates by gravity (e.g., centrifugation), or by magnetic separation (e.g., in case of using a magnetic bead). Following separation, the polypeptide-oligonucleotide conjugates and cDNA molecules undergo downstream processing in separate reactions.

The polypeptide-oligonucleotide conjugates are subjected to methods for polypeptide sequence detection (e.g., as described in Examples 2 and 3). For example, the polypeptide-oligonucleotide conjugates are used to generate first barcoded nucleic acid molecules. For example, a polypeptide-oligonucleotide conjugate is used to generate a first barcoded nucleic acid molecule comprising: 1) the partition-specific barcode or a complement thereof, and 2) a sequence corresponding to an amino acid sequence of the digested polypeptide of the polypeptide-oligonucleotide conjugate (e.g., one or more coding tag sequences).

The cDNA molecules and second nucleic acid barcode molecules are used to generate second barcoded nucleic acid molecules. For example, a cDNA molecule and a nucleic acid barcode molecule hybridized thereto are used to generate a second barcoded nucleic acid molecule comprising: 1) the partition-specific barcode or a complement thereof, and 2) a sequence of the cDNA molecule or a complement thereof. Generation of the second barcoded nucleic acid molecule can be performed via reverse transcription (e.g., in the case of the cDNA being directly hybridized to the second nucleic acid barcode molecule), or via ligation (e.g., in the case of the cDNA being indirectly hybridized to the second nucleic acid molecule via a splint molecule).

The first barcoded nucleic acid molecules (or derivatives thereof) and second barcoded nucleic acid molecules (or derivatives thereof) are sequenced. Analysis of the sequencing identifies sequences of the cDNAs and digested polypeptides, each associated with the partition-specific barcode, thereby identifying both the sequences and common cellular origin of the cDNAs and digested polypeptides. This workflow can be performed in parallel using a large number of partitions (e.g., droplets or wells) comprising single cells, thereby facilitating large-scale single-cell parallel analysis of protein/polypeptide and nucleic acid (e.g., mRNA) expression.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1-71. (canceled)

72. A method, comprising:
providing a plurality of digested polypeptides from a single cell in a partition, wherein the partition comprises a plurality of nucleic acid barcode molecules unique to the partition;
providing a reducing agent and a conjugation reagent to the partition to generate a plurality of polypeptide-oligonucleotide conjugates by coupling the plurality of digested polypeptides to the plurality of nucleic acid barcode molecules, wherein each polypeptide-oligonucleotide conjugate of the plurality of polypeptide-oligonucleotide conjugates comprises a digested polypeptide of the plurality of digested polypeptides coupled to a nucleic acid barcode molecule of the plurality of nucleic acid barcode molecules;
dispersing the plurality of polypeptide-oligonucleotide conjugates on a substrate; and
detecting, for a polypeptide-oligonucleotide conjugate of the plurality of polypeptide-oligonucleotide conjugates at a location on the substrate, a plurality of signals comprising (i) a first signal associated with the digested polypeptide of the polypeptide-oligonucleotide conjugate, and (ii) a second signal associated with the nucleic acid barcode molecule of the polypeptide-oligonucleotide conjugate.

73.-75. (canceled)

76. The method of claim 72, wherein, in each polypeptide-oligonucleotide conjugate of the plurality of polypeptide-oligonucleotide conjugates, the nucleic acid barcode molecule is covalently coupled to the digested polypeptide.

77.-87. (canceled)

88. The method of claim 72, wherein, in each polypeptide-oligonucleotide conjugate of the plurality of polypeptide-oligonucleotide conjugates, the nucleic acid barcode molecule is coupled to the digested polypeptide by copper-catalyzed alkyne-azide cycloaddition (CuAAC).

89.-95. (canceled)

96. The method of claim 72, further comprising lysing the single cell in the partition prior to providing the reducing agent and/or the conjugation reagent.

97. (canceled)

98. (canceled)

99. The method of claim 72, wherein, in each polypeptide-oligonucleotide conjugate of the plurality of polypeptide-oligonucleotide conjugates, the conjugation reagent facilitates coupling of the C-terminus of the digested polypeptide to an amine on the nucleic acid barcode molecule.

100. The method of claim 72, wherein a plurality of proteins is treated with an enzyme in the partition to provide the plurality of digested polypeptides.

101. (canceled)

102. (canceled)

103. The method of claim 72, wherein the plurality of nucleic acid barcode molecules is joined to a support, and the plurality of nucleic acid barcode molecules is releasable from the support upon application of a stimulus.

104.-106. (canceled)

107. The method of claim 72, wherein the generated polypeptide-oligonucleotide conjugates are pooled with additional generated polypeptide-oligonucleotide conjugates from a plurality of partitions, and the pooled polypeptide-oligonucleotide conjugates are dispersed on the substrate.

108. (canceled)

109. (canceled)

110. The method of claim 72, wherein a sequence of the digested polypeptide of the polypeptide-oligonucleotide conjugate and a sequence of the nucleic acid barcode molecule of the polypeptide-oligonucleotide conjugate are determined.

111. The method of claim 110, wherein determining the sequence of the digested polypeptide of the polypeptide-oligonucleotide conjugate comprises detecting a plurality of signals associated with the digested polypeptide of the polypeptide-oligonucleotide conjugate.

112. The method of claim 111, wherein determining the sequence of the digested polypeptide of the polypeptide-oligonucleotide conjugate comprises contacting the terminal amino acid of the digested polypeptide or a derivative thereof with a detectably labeled binding agent.

113.-116. (canceled)

117. The method of claim 110, wherein determining the sequence of the nucleic acid barcode molecule of the polypeptide-oligonucleotide conjugate comprises amplifying the sequence of the nucleic acid barcode molecule.

118. The method of claim 117, wherein the amplifying is performed by rolling circle amplification (RCA).

119. The method of claim 117, wherein the nucleic acid barcode molecule of the polypeptide-oligonucleotide conjugate on the substrate is contacted with a circularizable probe, wherein the circularizable probe comprises a 5′ hybridization region and a 3′ hybridization region that binds a first common sequence and a second common sequence, respectively, in the nucleic acid barcode molecule.

120. (canceled)

121. (canceled)

122. The method of claim 119, wherein the method comprises gap filling the circularizable probe using a cell barcode sequence of the nucleic acid barcode molecule of the polypeptide-oligonucleotide conjugate as a template, thereby incorporating a complement of the cell barcode sequence into the circularizable probe.

123. The method of claim 118, wherein RCA is performed by extending the 3′ end of the nucleic acid barcode molecule of the polypeptide-oligonucleotide conjugate on the substrate, wherein the 3′ end serves as a primer to generate an RCA product.

124. The method of claim 118, wherein the nucleic acid barcode molecule of the polypeptide-oligonucleotide conjugate or an extension thereof on the substrate is circularized and used as a template for RCA to generate an RCA product.

125. The method of claim 110, wherein determining the sequence of the digested polypeptide comprises:
contacting the digested polypeptide of the polypeptide-oligonucleotide conjugate with a binding agent comprising a coding tag sequence associated with the binding agent; and
extending the nucleic acid barcode molecule of the polypeptide-oligonucleotide conjugate with the coding tag sequence or a complement thereof, thereby generating an extended nucleic acid barcode molecule.

126.-128. (canceled)

129. The method of claim 125, wherein the extended nucleic acid barcode molecule identifies the digested polypeptide of the polypeptide-oligonucleotide conjugate and the single cell from which the digested polypeptide originated.

130. (canceled)

131. The method of claim 110, wherein determining the sequence of the nucleic acid barcode molecule of the polypeptide-oligonucleotide conjugate comprises performing sequencing by ligation, sequencing by synthesis, sequencing by binding, sequencing by avidity, or a combination thereof.

132.-243. (canceled)
